(19) United States
(12) Patent Application Publication
(10) Pub. No.: US 2016/0235073 A1
(43) Pub. Date: Aug. 18, 2016

(54) WHITE ANTIMICROBIAL COPPER ALLOY

(71) Applicant: SLOAN VALVE COMPANY, Franklin Park, IL (US)

(72) Inventors: Michael Murray, Franklin Park, IL (US); Mahi Sahoo, Ottawa (CA)

(73) Assignee: Sloan Valve Company, Franklin Park, IL (US)

(21) Appl. No.: 15/027,418

(22) PCT Filed: Oct. 7, 2014
(86) PCT No.: PCT/US14/59496

§ 371 Date: Apr. 5, 2016

Related U.S. Application Data
(60) Provisional application No. 61/887,765, filed on Oct. 7, 2013.

(51) Int. Cl. A01N 59/20; A61L 31/02; C22C 9/04

(52) U.S. Cl. CPC A01N 59/20; C22C 9/04; A61L 31/022; A61L 2300/404

(57) ABSTRACT

White/silver copper alloy that is machineable and have sufficient physical and mechanical properties for use in sand and permanent molding and casting. The alloys include less than 0.09 wt % lead to allow for use in potable water supplies. The alloys and also contains sufficient copper to exhibit antimicrobial properties for use in hospitals.

### CROSS REFERENCE TO RELATED APPLICATIONS

This application claims priority to U.S. Provisional Application No. 61/887,765 filed Oct. 7, 2013, reference of which is hereby incorporated in its entirety.

### FIELD OF THE INVENTION

The present invention generally relates to the field of alloys. Specifically, the embodiments of the present invention relate to copper alloys exhibiting a muted copper color, including, but not limited to rose, silver, white, or the like color which also have antimicrobial properties.

### BACKGROUND OF THE INVENTION

Copper alloys are used in many commercial applications. Many such applications involve the use of molds or casting to shape molten alloy into a rough form. This rough form may then be machined to the final form. Thus, the machinability of a copper alloy may be considered important. In addition, the other physical and mechanical properties such as ultimate tensile strength (“UTS”), yield strength (“YS”), percent elongation (“% E”), Brinell hardness (“BHN”), and modulus of elasticity (“MoE”) may be of varying degrees of importance depending on the ultimate application for the copper alloy.

One property imparted to copper alloys by copper is an antimicrobial effect. It is generally believed that alloys containing above 60% copper content will exhibit an antimicrobial effect. This antimicrobial effect appears to be through multiple pathways, making it very difficult for organisms to develop resistant strains.

Copper alloys, particularly copper alloys having high levels of copper typically exhibit a copper-like color. This color may not be desirable in the end product, such as due to consumer preferences or compatibility with other materials used in the end product.

Further, although copper imparts many useful properties to copper-based alloys, copper (and high copper alloys) are susceptible to tarnish. Exposed copper or a copper alloy surface can discolor and develop a patina. This may provide an undesirable visual characteristic.

Attempts have been made at developing a “white brass” that provides the color of white/silvery metals while retaining the properties of a brass alloy. Copper Development Association Registration Number C99700, known in the industry as white Tombasil™, is a leaded brass alloy that provides a somewhat silvery color. However, C99700 presents many problems. First, it relies upon a relatively high lead content (˜2%) to maintain the desirable machinability, a content considered significantly too high for commercial or residential water usage. Further, the alloy is difficult to machine, difficult to pour, and the intended silvery color is susceptible to discoloration (blackening).

As a result of the tendency of copper alloys to tarnish, many consumer goods that are made from copper alloys are painted or plated to provide a more appealing color and to prevent the detrimental effects of tarnish. One such example is plumbing fixtures. However, the needs and desire to plate a copper alloy also prevents the copper alloy from providing its antimicrobial effect, due to the surface of the consumer good being of the plated material rather than the underlying copper alloy.

### SUMMARY OF THE INVENTION

One embodiment of the invention relates to a white/silver copper alloy that is machinable and has sufficient physical properties for use in molding and casting. The alloy includes less than 0.09% lead to allow for use in water supplies and also contains sufficient copper to exhibit antimicrobial properties. Machinability of the white alloy remains very good despite the low lead content relative to prior commercial alloys.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a table listing commercial alloy compositions.

FIG. 2A is table listing a target C99761 alloy for sand casting corresponding actual test heats for same; FIG. 2B is a table for the target alloy of FIG. 2A listing the copper, nickel, zinc, sulfur, manganese, tin, antimony, and aluminum contents and the UTS, YS, % Elong, BHN, and Modulus of Elasticity for specific heats.

FIG. 3A is a table listing a first target C99761 alloy for permanent mold applications with corresponding actual test heats for same; FIG. 3B is a table for the target alloy of FIG. 3A listing the copper, nickel, zinc, sulfur, manganese, tin, antimony, and aluminum contents and the UTS, YS, % Elong, BHN, and Modulus of Elasticity for specific heats.

FIG. 4A is table listing a target C99771 alloy for sand casting and corresponding actual test heats for same; FIG. 4B is a table for the target alloy of FIG. 4A listing the copper, nickel, zinc, sulfur, manganese, tin, antimony, and aluminum contents and the UTS, YS, % Elong, BHN, and Modulus of Elasticity for specific heats.

FIG. 5A is a table listing a target C99771 alloy for permanent mold applications with corresponding actual test heats for same; FIG. 5B is a table for the target alloy of FIG. 5A listing the copper, nickel, zinc, sulfur, manganese, tin, antimony, and aluminum contents and the UTS, YS, % Elong, BHN, and Modulus of Elasticity for specific heats.

FIG. 6 is a free energy diagram of various sulfides, including antimony sulfide.

FIG. 7 is graph illustrating breakdown of antimony sulfide.

FIG. 8A illustrates a phase diagram of a variation of C99761 with no Sb under equilibrium conditions. FIG. 8B illustrates a phase diagram an embodiment of C99761 with 0.6 wt % Sb. FIG. 8C is a phase assemblage diagram of an embodiment of C99761 with no Sb under equilibrium conditions. FIG. 8D is a magnified phase assemblage diagram of a variation of C99761 with no Sb; FIG. 8E is a phase assemblage diagram of C99761 with 0.6 Sb. FIG. 8F is a magnified phase assemblage diagram of C99761 with 0.6 Sb. FIG. 8G is a phase assemblage diagram of a variation of C99761 with no Sb-Scheil Cooling. FIG. 8H is a phase assemblage diagram of C99761 with 0.6 Sb-Scheil Cooling.

FIG. 9A illustrates a phase diagram of an embodiment of C99771 under equilibrium conditions; FIG. 9B illustrates a phase diagram an embodiment of C99771 with 0.6 wt % Sb. FIG. 9C is a phase assemblage diagram of an embodiment of C99771 with no Sb under equilibrium conditions. FIG. 9D is a magnified phase assemblage diagram of a variation of C99771 with no Sb; FIG. 9E is a phase assemblage diagram of C99771 with 0.6 Sb. FIG. 9F is a magnified phase assemblage diagram of C99771 with 0.6 Sb. FIG. 9G is a phase assemblage diagram of a variation of C99771 with no Sb-Scheil Cooling. FIG. 9H is a phase assemblage diagram of C99771 with 0.6 Sb-Scheil Cooling.

FIG. 10A is a table listing the C99761 dezincification formulation utilized for the testing illustrated in FIGS. 10B-C; FIG. 10B illustrates dezincification corrosion to a max depth (horizontal line) of 0.0002 inches (5.1 microns) from the exposed surface (horizontal top) in the thin section of the metallographic section; FIG. 10C illustrates no significant dezincification corrosion in the thick section of a metallographic section.

FIG. 11A is a table listing the C99771 dezincification formulation utilized for the testing illustrated in FIGS. 11B-11C. 11B illustrates dezincification corrosion testing showing a maximum depth (red line) of 0.0002″ (5.1 microns) from the exposed surface (horizontal top) in the metallographic thin section prepared from the submitted sample in the transverse orientation. Unetched. (494×). FIG. 11C illustrates dezincification corrosion testing showing to a maximum depth (red line) of 0.0002″ (5.1 microns) from the exposed surface (horizontal top) in the metallographic thick section prepared from the submitted sample in the longitudinal orientation.

FIG. 12A is a table indicating the composition of an embodiment of sand-cast alloy C99761 (62.6 Cu, 8.17 Ni, 16.94 Zn, 10.36 Mn, 0.012 S, 0.492 Sb, 0.882 Sn, 0.126 Fe, 0.350 Al, 0.040 P, 0.009 Pb, 0.002 Si, 0.002 C); FIG. 12B is a micrograph; FIG. 12C is a BE image showing annotated locations and corresponding EDS spectra.

FIG. 13A is a SEM image of an embodiment of alloy C99761; FIG. 13B illustrates elemental mapping of sulfur in the portion shown in FIG. 13A; FIG. 13C illustrates elemental mapping of phosphorous in the portion shown in FIG. 13A; FIG. 13D illustrates elemental mapping of zinc in the portion shown in FIG. 13A; FIG. 13E illustrates elemental mapping of copper in the portion shown in FIG. 13A; FIG. 13F illustrates elemental mapping of manganese in the portion shown in FIG. 13A; FIG. 13G illustrates elemental mapping of tin in the portion shown in FIG. 13A; FIG. 13H illustrates elemental mapping of antimony in the portion shown in FIG. 13A;

FIG. 14A is a backscatter electron image of an alloy of C99761 sand cast of FIG. 12A (200×); FIG. 14B is a backscatter electron image of an alloy of C99761 sand cast of FIG. 12A (1000×); FIG. 14C is a micrograph of a sample C99761 sand cast of FIG. 12A (500×).

FIG. 15A is a table indicating the composition of an embodiment of sand-cast alloy C99771 (69.2 Cu, 3.21 Ni, 8.10 Mn, 17.56 Zn, 0.014 S, 0.685 Sb, 0.319 Fe, 0.616 Sn, 0.006 Pb, 0.224 Al); FIG. 15B is a micrograph; FIG. 15C BE image showing annotated locations and corresponding EDS spectra.

FIG. 16A is a SEM image of an embodiment of alloy C99771; FIG. 16B illustrates elemental mapping of phosphorous in the portion shown in FIG. 16A; FIG. 16C illustrates elemental mapping of sulfur in the portion shown in FIG. 16A; FIG. 16D illustrates elemental mapping of zinc in the portion shown in FIG. 16A; FIG. 16E illustrates elemental mapping of copper in the portion shown in FIG. 16A; FIG. 16F illustrates elemental mapping of manganese in the portion shown in FIG. 16A;

FIG. 16G illustrates elemental mapping of tin in the portion shown in FIG. 16A; FIG. 16H illustrates elemental mapping of antimony in the portion shown in FIG. 16A;

FIG. 17A is a backscatter electron image of an alloy of C99771 sand cast of FIG. 15A (200×); FIG. 17B is a backscatter electron image of an alloy of C99771 sand cast of FIG. 15A (1000×); FIG. 17C is a micrograph of a sample C99771 sand cast of FIG. 15A (500×)..

FIG. 18A is a table indicating the composition of an embodiment of alloy C99761 for permanent mold casting; FIGS. 18B and 18C are backscattered electron image of the C99761 alloy of FIG. 18A at 200× and 1000× respectively; FIG. 18D is a micrograph of the C99761 alloy of FIG. 18A alloy (500×).

FIG. 19A is a micrograph of the C99761 alloy of FIG. 18A at 5000× magnification annotated with 5 marked regions; FIG. 19B-F are EDS spectra corresponding to annotated locations 1-5, respectfully, of FIG. 19A.

FIG. 20A is a SEM image of the C99761 alloy of FIG. 18A; FIG. 20B illustrates elemental mapping of copper in the portion shown in FIG. 20A; FIG. 20C illustrates elemental mapping of manganese in the portion shown in FIG. 20A; FIG. 20D illustrates elemental mapping of lead in the portion shown in FIG. 20A; FIG. 20E illustrates elemental mapping of tin in the portion shown in FIG. 20A; FIG. 20F illustrates elemental mapping of zinc in the portion shown in FIG. 20A; FIG. 20G illustrates elemental mapping of nickel in the portion shown in FIG. 20A; FIG. 20H illustrates elemental mapping of aluminium in the portion shown in FIG. 20A; FIG. 20I illustrates elemental mapping of antimony in the portion shown in FIG. 20A.

FIG. 21A is a table indicating the composition of an embodiment of alloy C99771 for permanent mold casting; FIGS. 21B and 21C are backscattered electron images of the C99771 alloy of FIG. 21A (200× and 1000× respectively.); FIG. 21D is a micrograph of the C99771 alloy of FIG. 21A alloy (500×).

FIG. 22A is a micrograph of the C99771 alloy of FIG. 21A at 5000× magnification annotated with 5 marked regions; FIG. 22B-F are EDS spectra corresponding to annotated locations 1-5, respectfully, of FIG. 22A.

FIG. 23A is a SEM image of the C99761 alloy of FIG. 21A; FIG. 23B illustrates elemental mapping of copper in the portion shown in FIG. 23A; FIG. 23C illustrates elemental mapping of manganese in the portion shown in FIG. 23A; FIG. 23D illustrates elemental mapping of lead in the portion shown in FIG. 23A; FIG. 23E illustrates elemental mapping of tin in the portion shown in FIG. 23A; FIG. 23F illustrates elemental mapping of nickel in the portion shown in FIG. 23A; FIG. 23G illustrates elemental mapping of zinc in the portion shown in FIG. 23A; FIG. 23H illustrates elemental mapping of aluminium in the portion shown in FIG. 23A; FIG. 23I illustrates elemental mapping of antimony in the portion shown in FIG. 23A.

FIG. 24A is a table listing heat compositions of a C99761 sand cast alloy used for mechanical property testing; FIG. 24B is a graph of mechanical properties for the sand cast alloy of C99761 in FIG. 24A;

FIG. 25A is a table listing heat compositions of a C99761 permanent mold alloy used for mechanical property testing FIG. 25B is a graph of mechanical properties for the permanent mold alloy of C99761 in FIG. 25A;

FIG. 26A is the composition of a C99771 sand cast alloy used for mechanical property testing; FIG. 26B is a graph of mechanical properties for the sand cast alloy of C99771 in FIG. 26A;

FIG. 27A is the composition of a C99771 permanent mold alloy used for mechanical property testing; FIG. 27B is a graph of mechanical properties for the permanent mold alloy of C99771 in FIG. 27A;

FIG. 28 illustrates a graph comparing machinability of C99761 alloys and C99771 alloys to other known alloys (by CDA registration number).

FIG. 29A illustrates chips from a machinability test of embodiments of C99761 (99761-091113-P14H8-1 with 61.72 Cu, 8.80 Ni, 16.69 Zn, 10.69 Mn, 0.011 S, 0.732 Sb, 0.736 Sn, 0.245 Fe, 0.305 Al, 0.044 P, 0.009 Pb, 0.002 Si and 0.002 C); FIGS. 29B-E illustrate chip morphology of alternative implementations of C99761 alloy.

FIG. 30A-E illustrates chips from a machinability test of embodiments of C99771 (999771-082713-P11H19-1 with 65.04 Cu, 3.04 Ni, 19.30 Zn, 10.63 Mn, 0.004 S, 0.675 Sb, 0.776 Sn, 0.177 Fe, 0.291 Al, 0.046 P, 0.008 Pb, 0.002 Si, 0.001 C); FIGS. 30B-E illustrate chip morphology of alternative implementations of C99771 alloy.

FIG. 31A illustrates a composition similar to those of FIGS. 30A-E but lacking antimony and FIG. 31B illustrates chip morphology for the composition of FIG. 31A

FIG. 32 is a graph of color comparison data for C99761 and C99771 with a chrome plated part as reference.

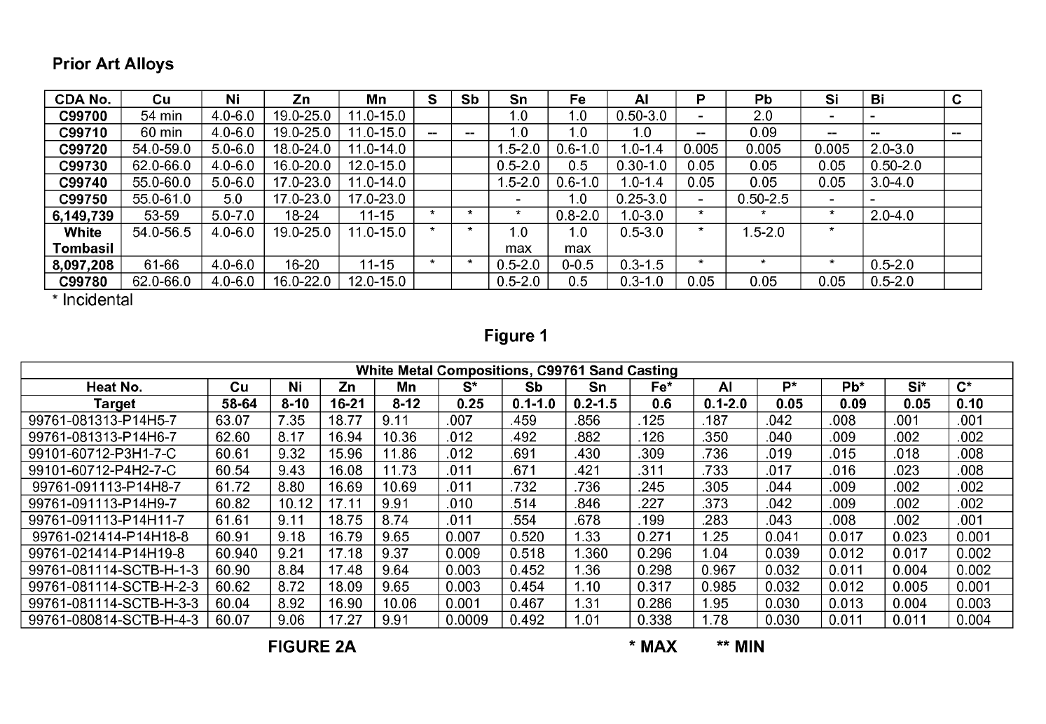

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

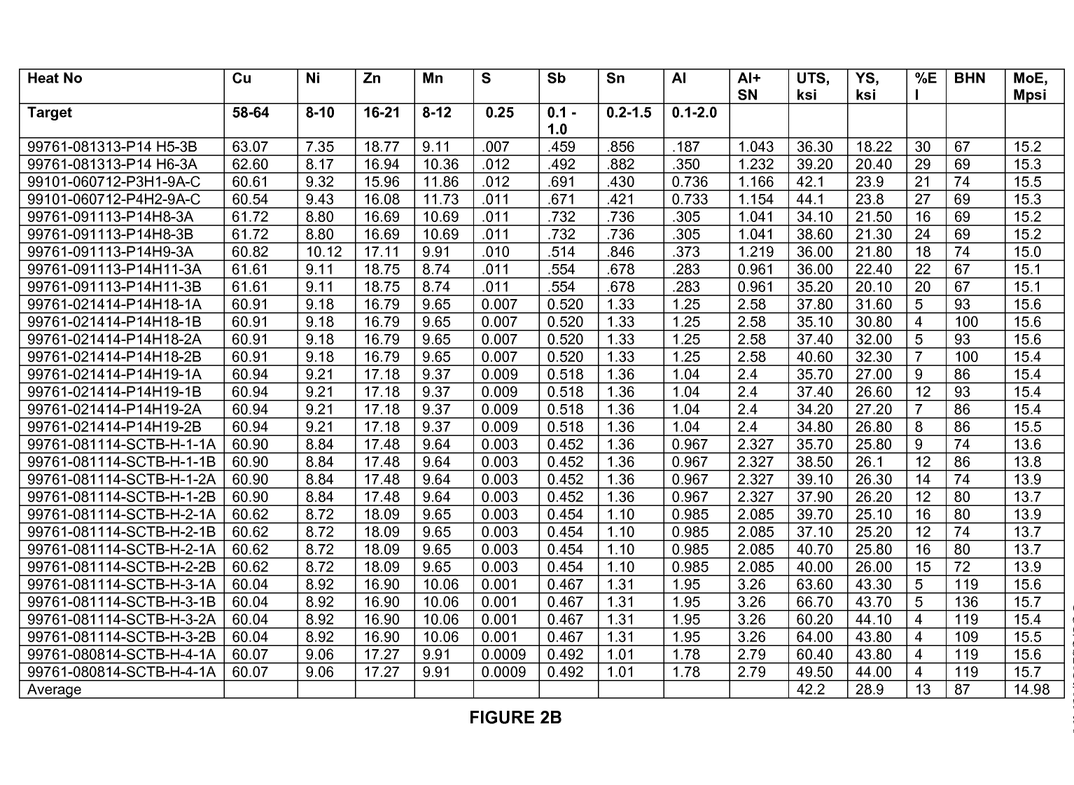

Various embodiments of two alloys, referred to as C99761 and C99771 for ease of reference, as set forth in the tables of FIGS. 2A (C99761 Sand Cast), 3A (C99761 Permanent Mold), 4A (C99771 Sand Cast), and 5A (C99771 Permanent Mold) are described herein. Two separate target compositions for each of the C99761 and C99771 alloys for each respective of sand cast and permanent mold is described in the referenced figures. The described alloys are antimicrobial. Both alloys utilize a relatively low amount of copper comparative to prior art alloys that provide antimicrobial features. The alloys provide for ease of recycling due to the absence or mere trace amounts of certain undesirable elements such as bismuth. The mixing of Bi chips with other no-lead alloys causes cracking issues in the wrought alloys. When machining alloys with bismuth any contamination of bismuth chips reduces the value of the contaminated chips by as much as 33%, which adds to the cost of the products produced.

The melting points of the alloys are relatively low compared to prior art alloys useful in similar applications. The lower melting point will allow for a lower cost of product. The alloys also provide a finish and color that negates the need for chrome plating, resulting in a more environmentally friendly production.

One embodiment relates to compositions of a copper alloy that contain a sufficient amount of copper to exhibit an antimicrobial effect, an average wt % copper preferably more than 60%. The copper alloy may be a brass comprising, in addition to the copper, the following: zinc, nickel, manganese, sulfur, iron, aluminum, tin, antimony. The copper alloy may further contain small amounts of phosphorous, lead, and carbon. Preferably, the copper alloy contains no lead or less than 0.09% lead, so as to reduce the deleterious impact of leaching in potable water applications. In one embodiment, the alloy provides less than 0.09% lead while including at least 60% copper to impart antimicrobial properties and provides a machineable final product with a final color and gloss that is substantially equivalent to that of traditional plated red-brass alloys, i.e. a white or silvery color generally associated with nickel or chrome plating. In one implementation, the as-cast color of the alloy is a gray color, but after buffing and or polishing a silver white brilliance can be obtained. However the gray as cast condition will be, in certain applications, beneficial as this will identify this alloy as being low lead, and visually different from other leaded alloys and low lead alloys. This factor will help in the future identification for sorting and remelting of alloys in the scrap stream.

The copper alloys of one embodiment of the present invention provide a white/silver color. This color and the antimicrobial aspect of the alloy's surface make plating of products made from the alloy unnecessary. The avoidance of the need for plating of brass alloys provides opportunities for a substantially reduced environmental footprint. Extensive energy is necessary for the electroplating process commonly used and the process also involves the use of harsh chemicals.

### Alloy Compositions

As noted above, presently described are an alloy group C99761 and a second alloy group C99771. All percentage ranges for compositions noted herein are weight percentage.

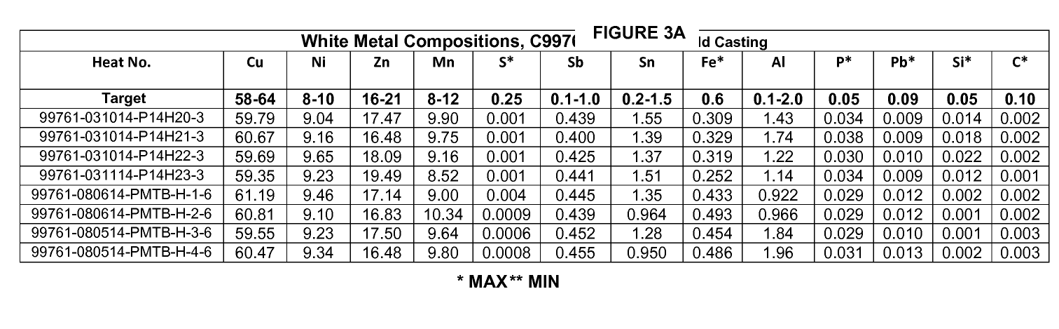

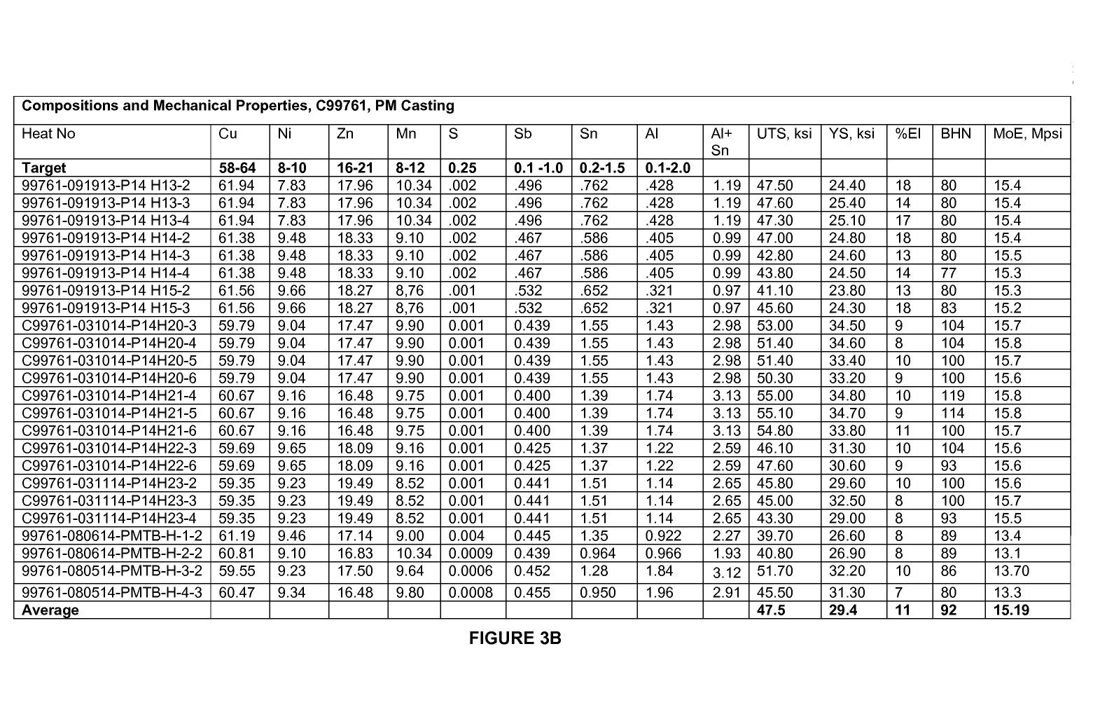

One embodiment of an alloy, includes about 60% minimum copper, about 8-10% nickel, about 16-21% zinc, about 8-12% manganese, about 0.25% sulfur, about 0.1%-1% antimony, about 0.2%-1.5% tin. In a further embodiment, the alloy includes one or more of about 0.6% iron, about 0.1-2.0% aluminum, about 0.1% carbon, about 0.05% phosphorous, less than 0.09% lead, and less than 0.05% silicon. Such embodiment is generally referred to herein as C99761 alloy and is, for example, the target formulation for the heats listed in FIGS. 2A and 3A.

The first alloy group 99761 provides a target alloy for sand casting comprising a balance of copper of 58-64 wt % with: 8-10 wt % nickel, 16-21 wt % zinc, 8-12 wt % manganese, greater than 0 and less than 0.25 wt % sulfur, 0.1 to 1.0 wt % antimony, 0.2 to 1.5 wt % tin, greater than 0 and less than 0.6 wt % iron, 0.1 to 2.0 wt % aluminum. This target C99761 sand cast alloy may further comprise greater than 0 and less than 0.05 wt % phosphorous, less than 0.09 wt % lead, greater than 0 and less than 0.05 wt % silicon, and greater than 0 and less than 0.1 wt % carbon.

The second alloy group 99761 provides a target alloy for permanent mold casting comprising copper of at least 58 to 64 wt % with: 8-10 wt % nickel, 16-21 wt % zinc, 8-12 wt % manganese, greater than 0 and less than 0.25 wt % sulfur, 0.1 to 1.0 wt % antimony, 0.2 to 1.5 wt % tin, greater than 0 and less than 0.6 wt % iron, 0.1 to 2.0 wt % aluminum. This target C99761 permanent mold alloy may further comprise greater than 0 and less than 0.05 wt % phosphorous, less than 0.09 wt % lead, greater than 0 and less than 0.05 wt % silicon, and greater than 0 and less than 0.1 wt % carbon.

For both the sand cast and permanent mold embodiments described above, the aluminium content may be selected to be greater than 0.2% in one specific implementation to improve the mechanical properties for certain applications such as plumbing valves. The preferred amount of Sn plus Al is 1.8%, most preferable as 0.8% Sn and 1% Al.

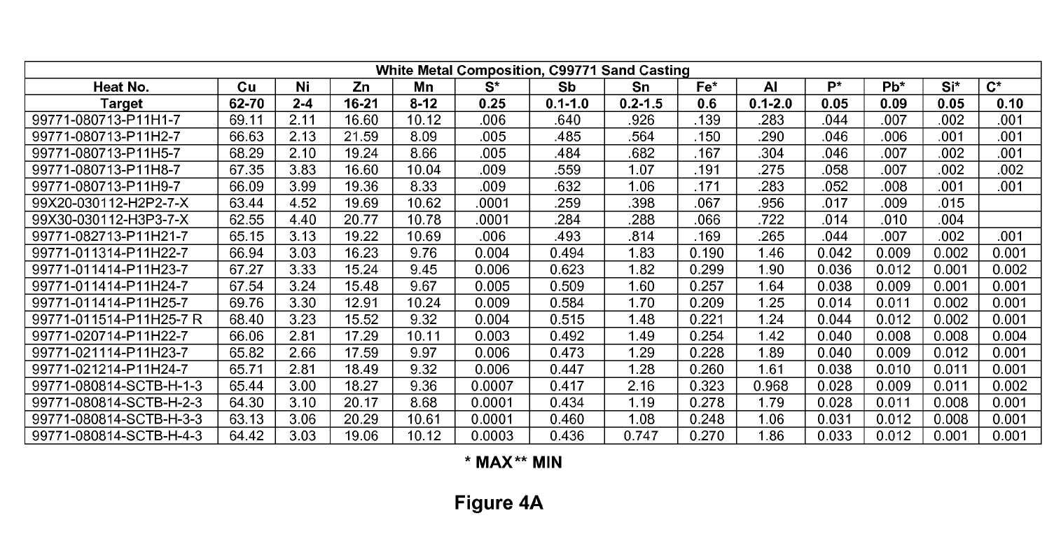

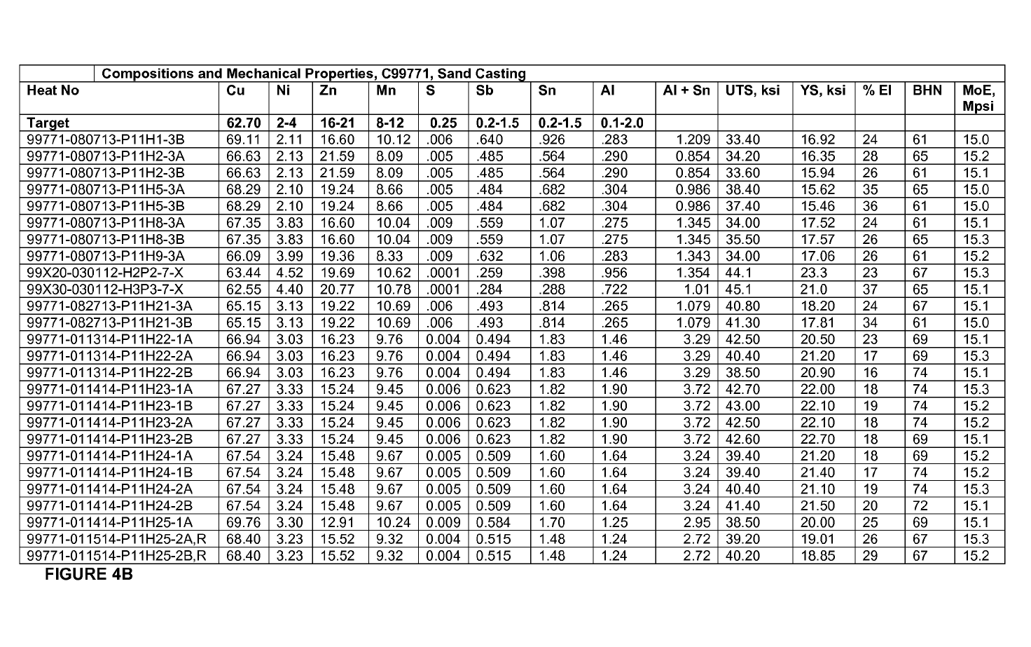

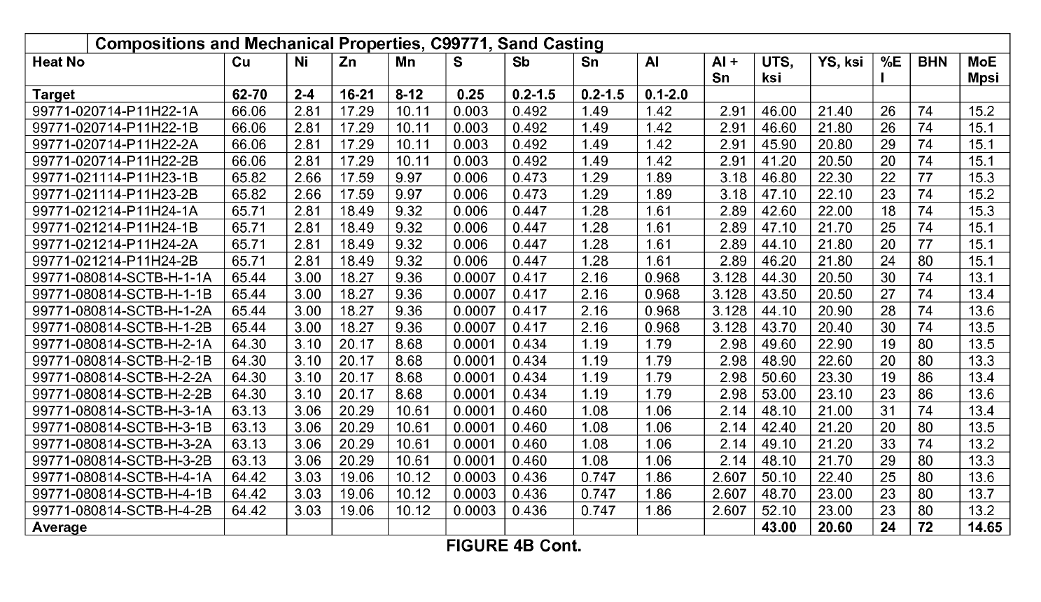

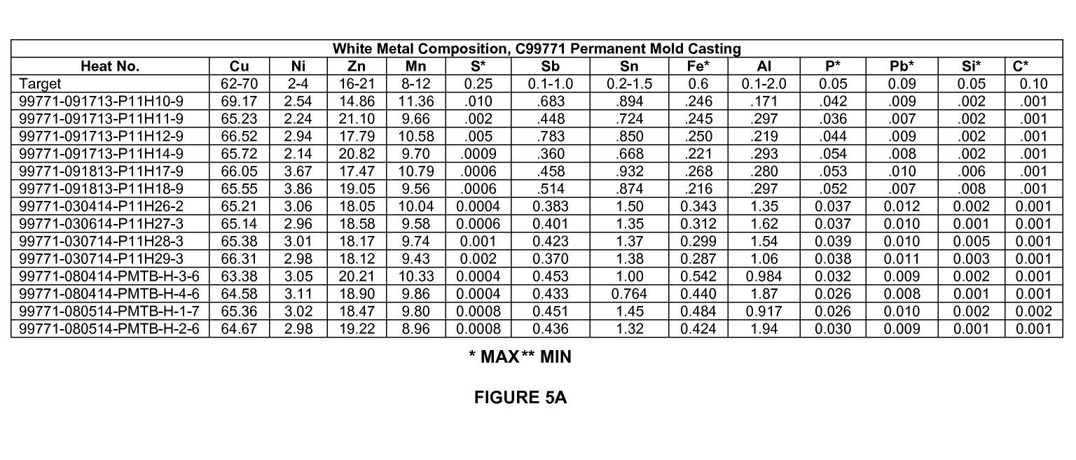

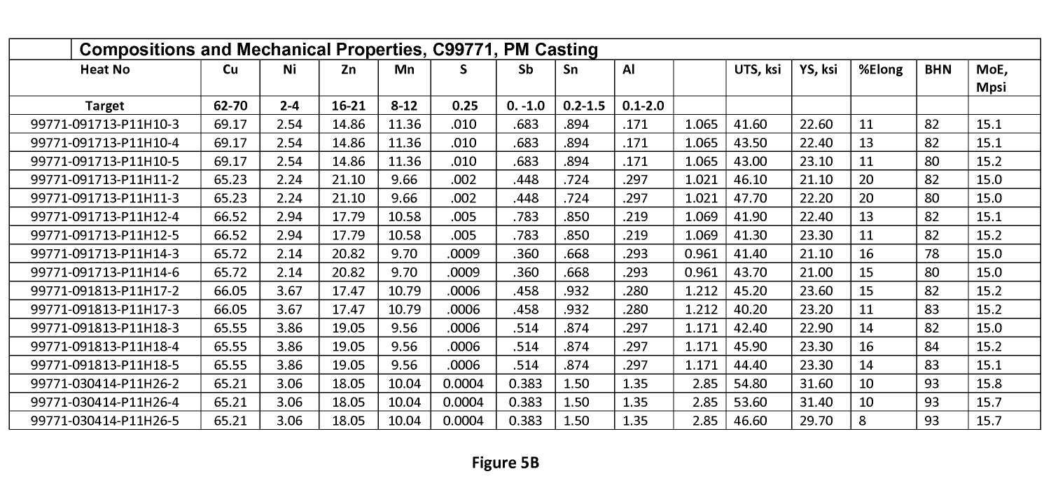

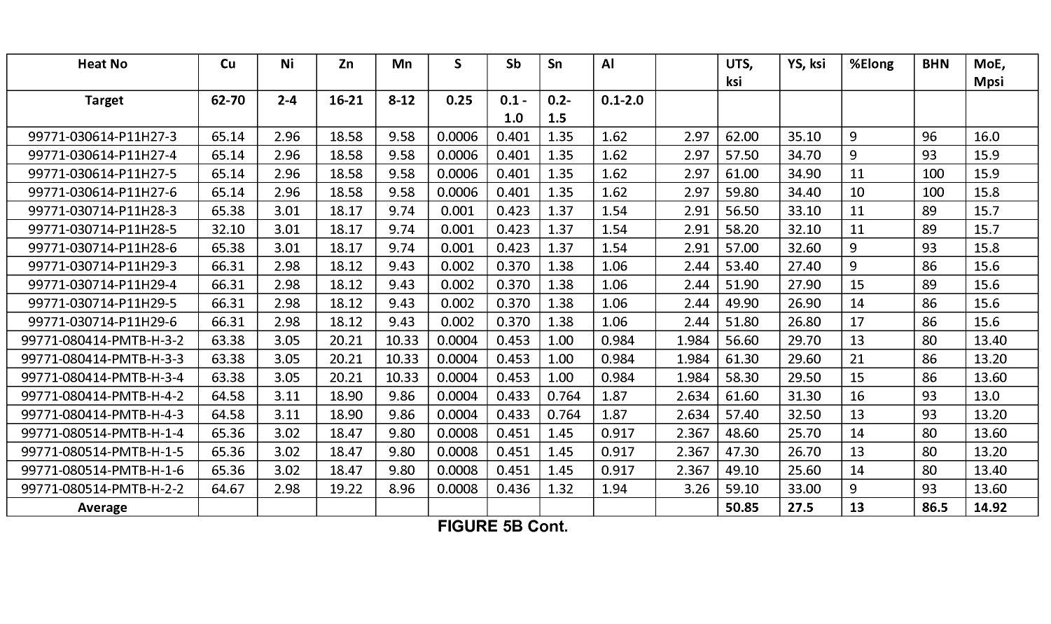

One embodiment of an alloy, includes about 62-70% minimum copper, about 2-4% nickel, about 16-21% zinc, about 8-12% manganese, about 0.25% sulfur, about 0.1%-1% antimony, about 0.2%-1.5% tin. In a further embodiment, the alloy includes one or more of about 0.6% iron, about 0.1-2.0% aluminum, about 0.1% carbon, about 0.05% phosphorous, less than 0.09% lead, and less than 0.05% silicon. Such embodiment is generally referred to herein as C99771 alloy and is, for example, the target formulation for the heats listed in FIGS. 4A and 5A.

The first alloy group 99771 provides a target alloy for sand casting comprising copper of at least 62 to 70 wt % with: 2-4 wt % nickel, 16-21 wt % zinc, 8-12 wt % manganese, greater than 0 and less than 0.25 wt % sulfur, 0.1 to 1.0 wt % antimony, 0.2 to 1.5 wt % tin, greater than 0 and less than 0.6 wt % iron, 0.1 to 2.0 wt % aluminum. This target C99771 sand cast alloy may further comprise greater than 0 and less than 0.05 wt % phosphorous, less than 0.09 wt % lead, greater than 0 and less than 0.05 wt % silicon, and greater than 0 and less than 0.1 wt % carbon.

The second alloy group 99771 provides a target alloy for permanent mold casting comprising copper of at least 62 to 70 wt % with: 2-4 wt % nickel, 16-21 wt % zinc, 8-12 wt % manganese, greater than 0 and less than 0.25 wt % sulfur, 0.1 to 1.0 wt % antimony, 0.2 to 1.5 wt % tin, greater than 0 and less than 0.6 wt % iron, 0.1 to 2.0 wt % aluminum. This target C99771 permanent mold cast alloy may further comprise greater than 0 and less than 0.05 wt % phosphorous, less than 0.09 wt % lead, greater than 0 and less than 0.05 wt % silicon, and greater than 0 and less than 0.1 wt % carbon.

For both the sand cast and permanent mold embodiments described above, the aluminium content may be selected to be greater than 0.2% in one specific implementation to improve the mechanical properties for certain applications such as plumbing valves. In one embodiment, the Sn+Al is 1.8 wt %, most preferably with about 0.8% Sn and 1% Al.

Alloys of the present invention exhibit a balance of several desirable properties and exhibit superior characteristics and performance to prior art alloys. FIGS. 2B and 3B are tables providing the UTS, YS, % Elong, BHN, and Modulus of Elasticity for several heats of C99761 alloys of the present invention. FIGS. 4B and 5B are tables providing the UTS, YS, % Elong, BHN, and Modulus of Elasticity for several heats of C99771 alloys of the present invention.

The alloys, comprise as a principal component, copper. Copper provides basic properties to the alloy, including antimicrobial properties and corrosion resistance. Pure copper has a relatively low yield strength, and tensile strength, and is not very hard relative to its common alloy classes of bronze and brass. Therefore, it is desirable to improve the properties of copper for use in many applications through alloying. The copper will typically be added as a base ingot. The base ingot's composition purity will vary depending on the source mine and post-mining processing. The copper may also be sourced from recycled materials, which can vary widely in composition. Therefore, the alloys of the present invention may have certain trace elements without departing from the spirit and scope of the invention. Further, it should be appreciated that ingot chemistry can vary, so, in one embodiment, the chemistry of the base ingot is taken into account. For example, the amount of zinc in the base ingot is taken into account when determining how much additional zinc to add to arrive at the desired final composition for the alloy. The base ingot should be selected to provide the required copper for the alloy while considering the secondary elements in the base ingot and their intended presence in the final alloy since small amounts of various impurities are common and have no material effect on the desired properties.

It is believed that the presence of a high amount of zinc increases the strength and hardness but reduces ductility by solid solution strengthening and by forming Cu—Zn intermetallic phase such as Cu₃Zn. It also increases the solidification range. Casting fluidity increases with zinc content. It is believed that the presence of Zn is similar to that of Sn but to a lesser degree, in certain embodiments approximately 2% Zn is roughly equivalent to 1% Sn with respect to the above mentioned improvements to characteristics noted. Zn is known, in sufficient quantities, to cause copper to be present in beta rather than alpha phase. The beta phase results in a harder material, thus Zn increases strength and hardness by solid solution hardening. However, Cu—Zn alloys have a short freezing range. Zinc has traditionally been less expensive than tin and, thus, used more readily. Zinc above a certain amount, typically about 14%, can result in an alloy susceptible to dezincification. In addition, it has been discovered that higher amounts of zinc prevent the sulfur from integrating into the melt. It is believed that some Zn remains in solid solution with Cu. Some Zn is associated with some intermetallic phases. The rest reacts with S to form ZnS. In one embodiment, the C99761 and C99771 alloys comprise 16% to 21% Zn. The deleterious impact of this amount of zinc, such as dezincification susceptibility, is mitigated by the other constituents in the alloy, notably the antimony. Thus, the C99761 and C99771 alloys exhibit beneficial properties associated with the higher zinc content while minimizing the drawbacks exhibited by prior art alloys. Many elements are referred to in terms of “zinc equivalents” as discussed below with regard to the relative impact of the element compared to zinc.

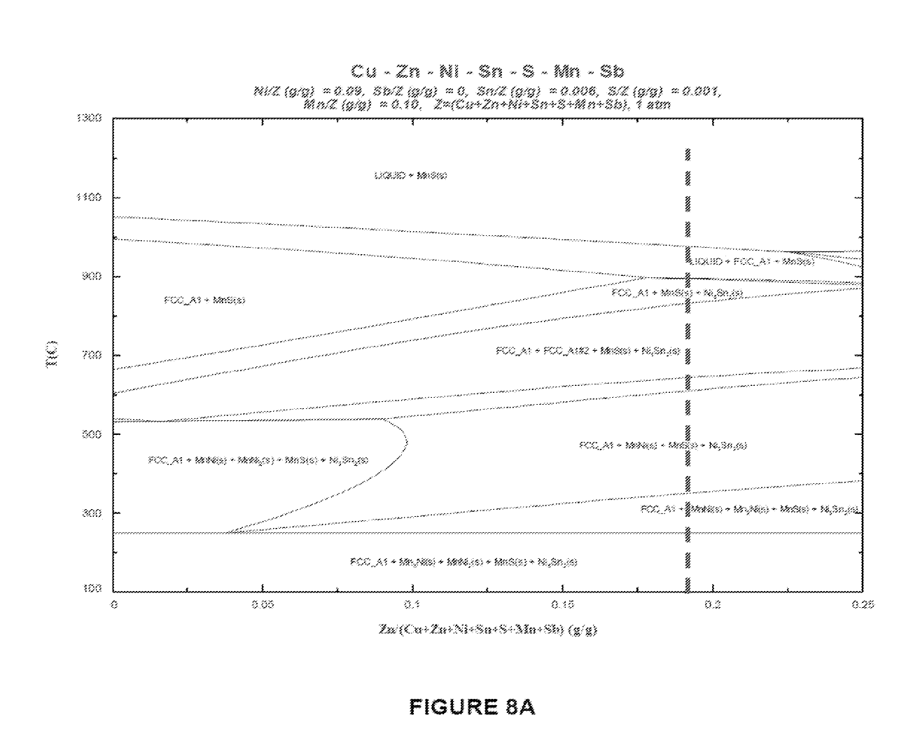

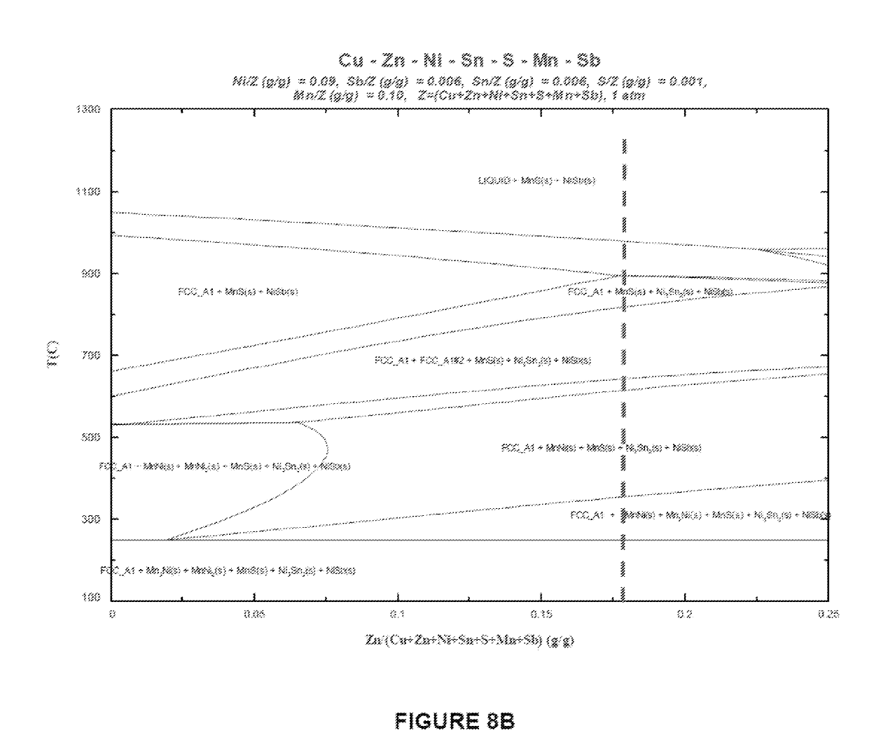

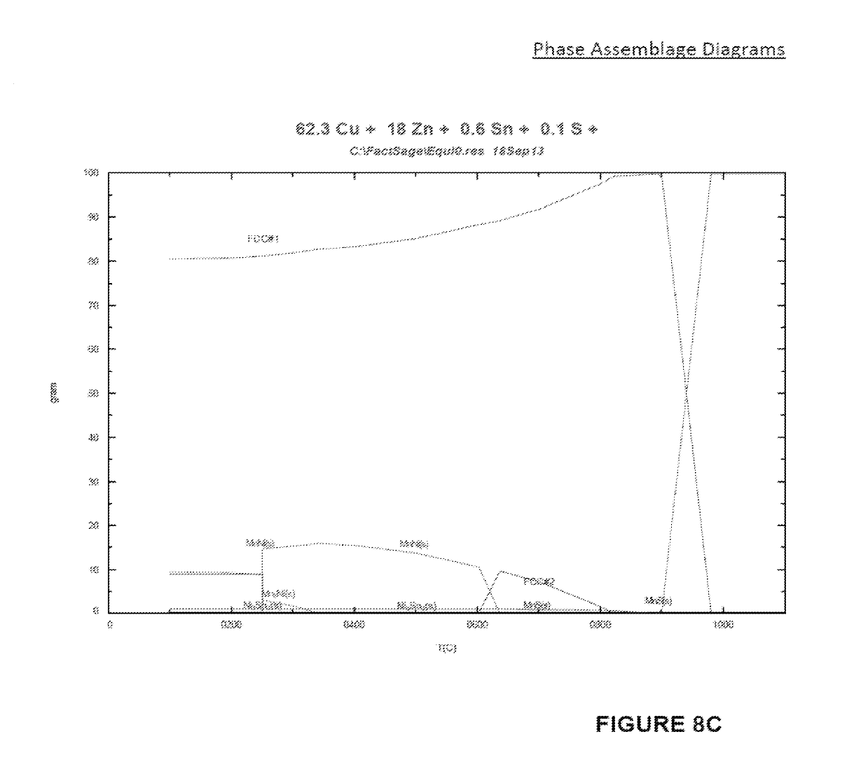

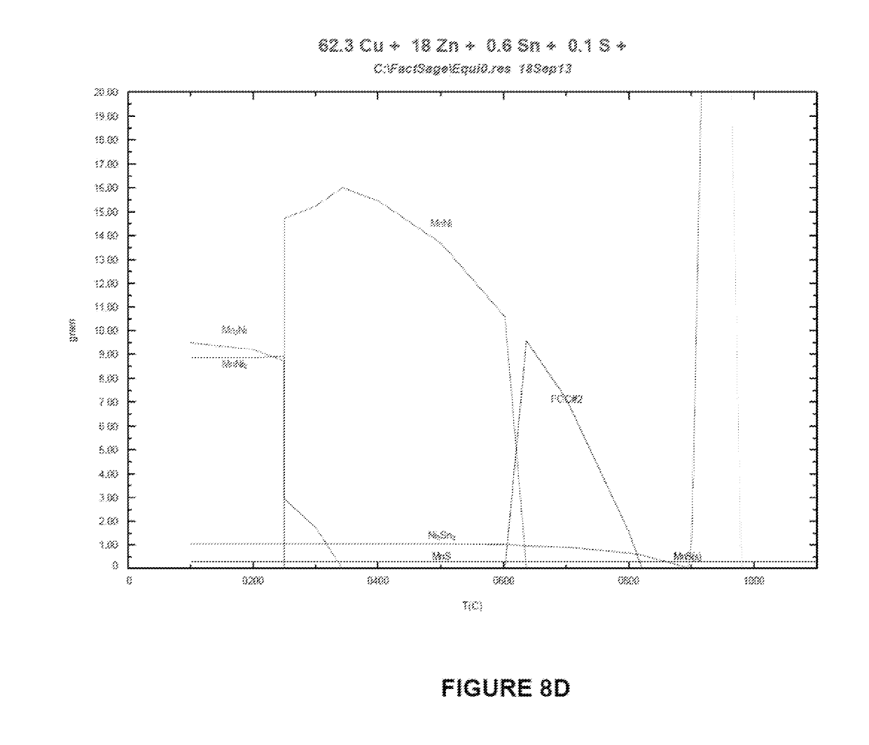

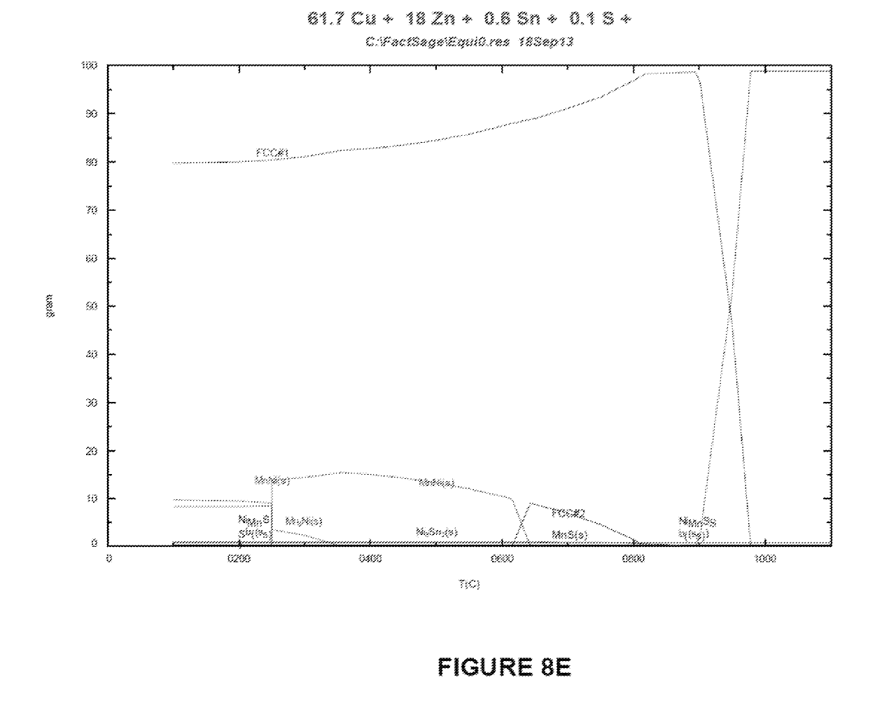

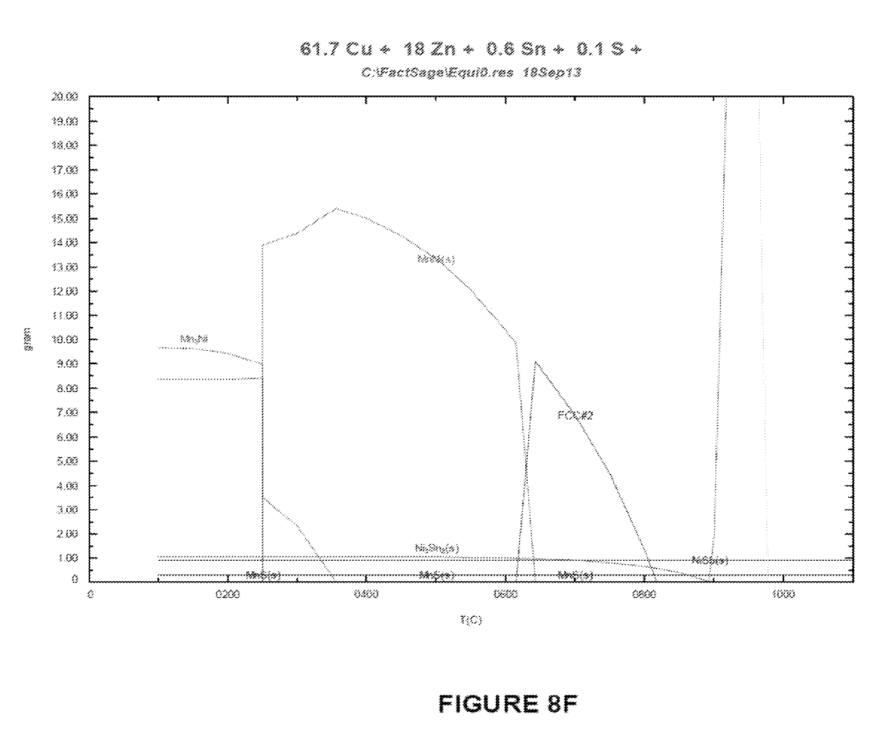

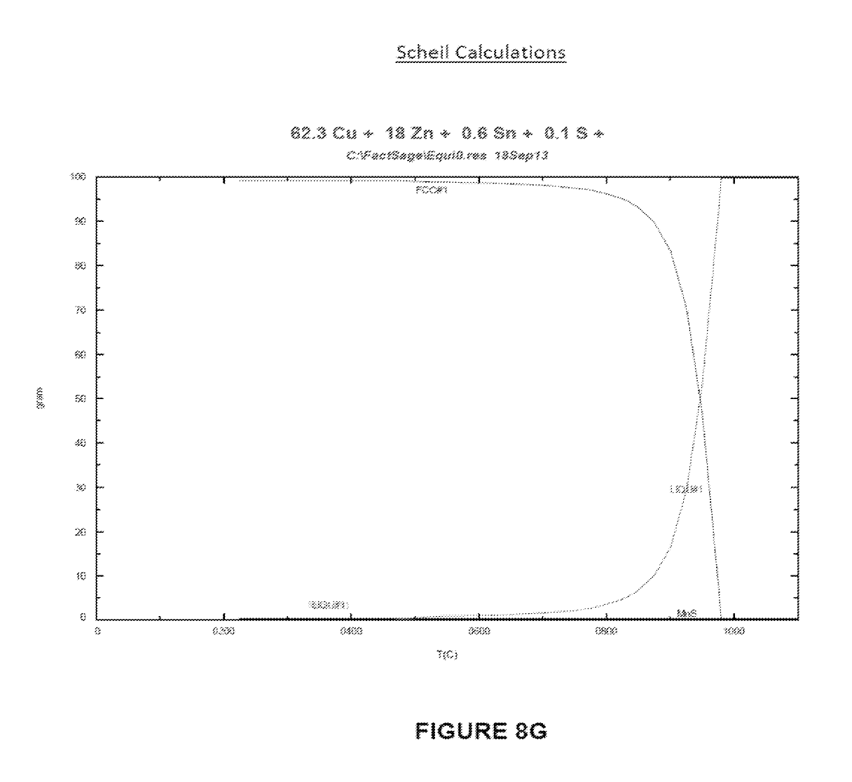

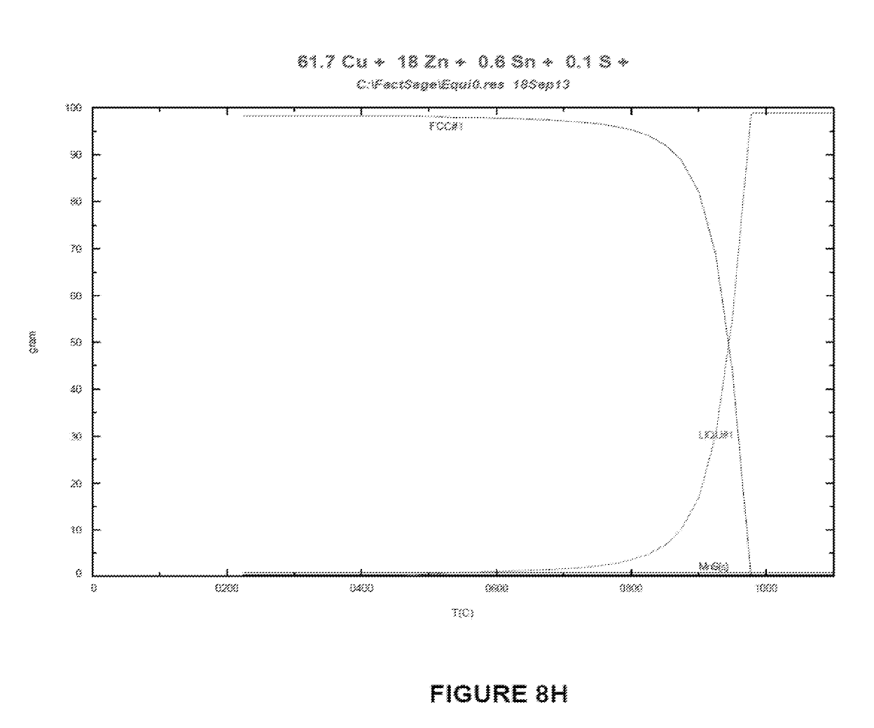

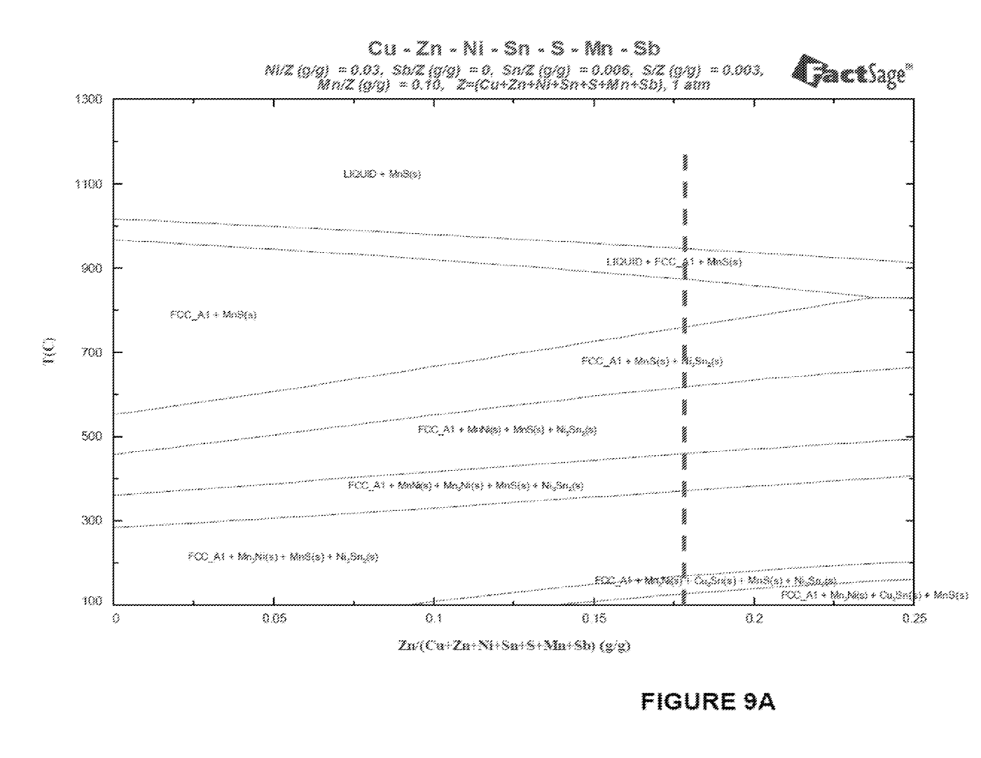

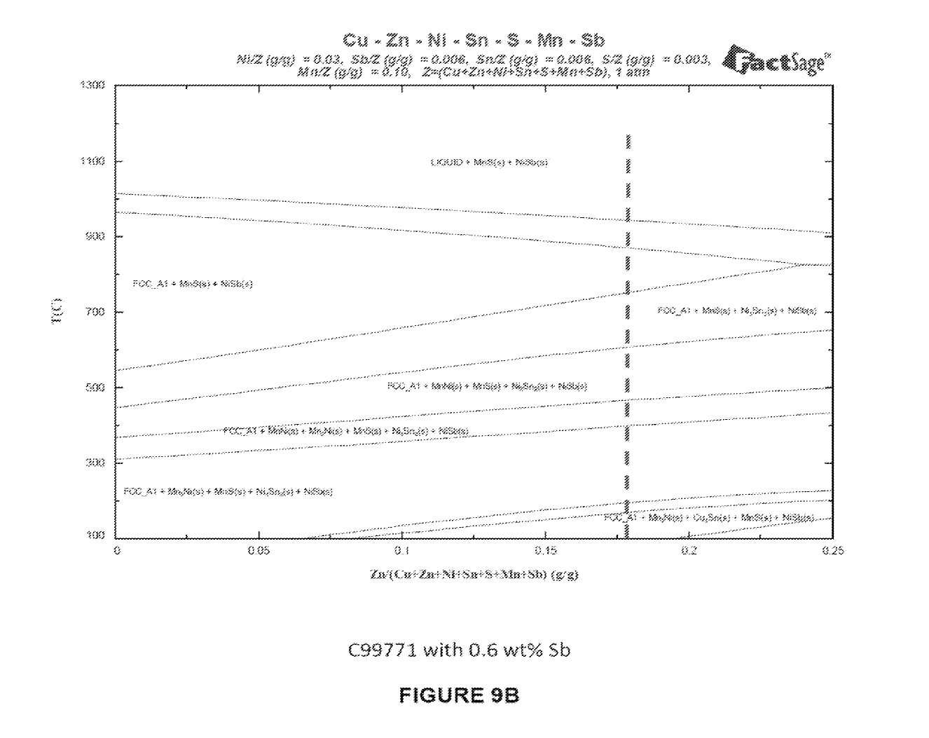

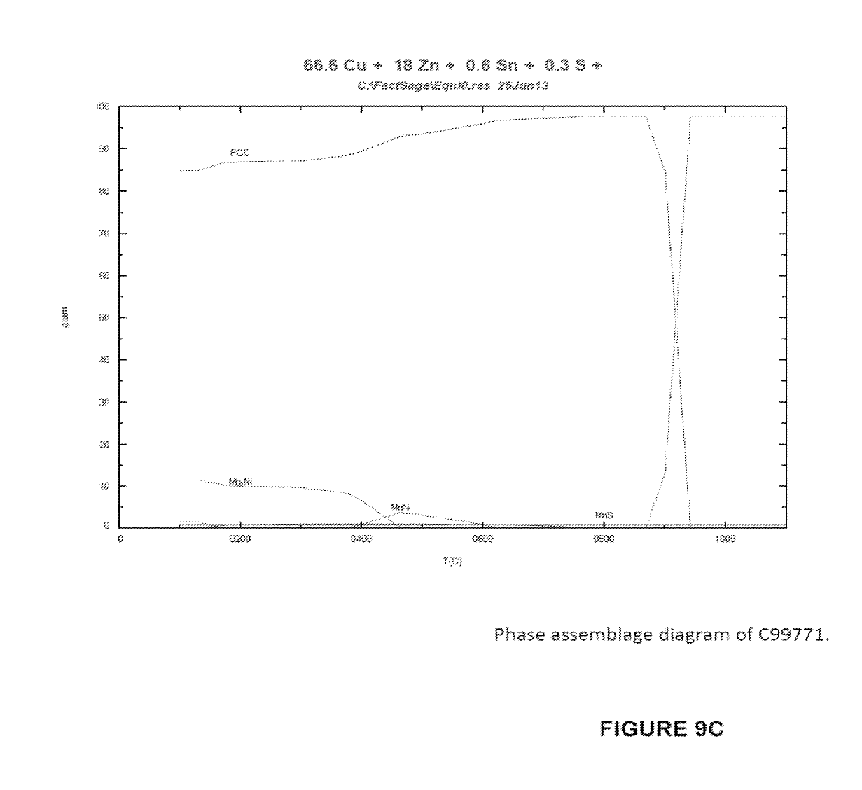

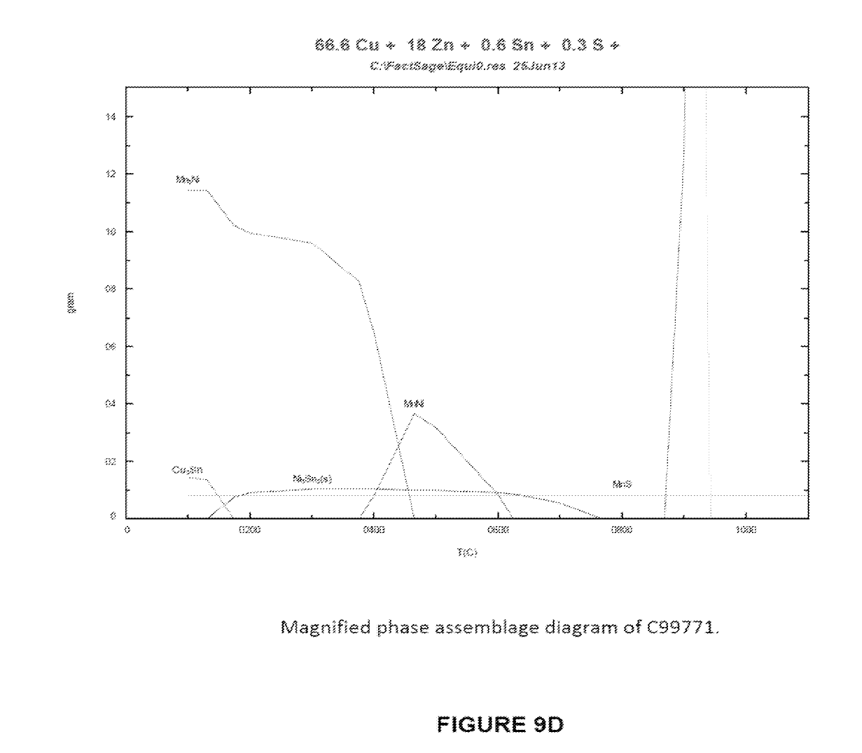

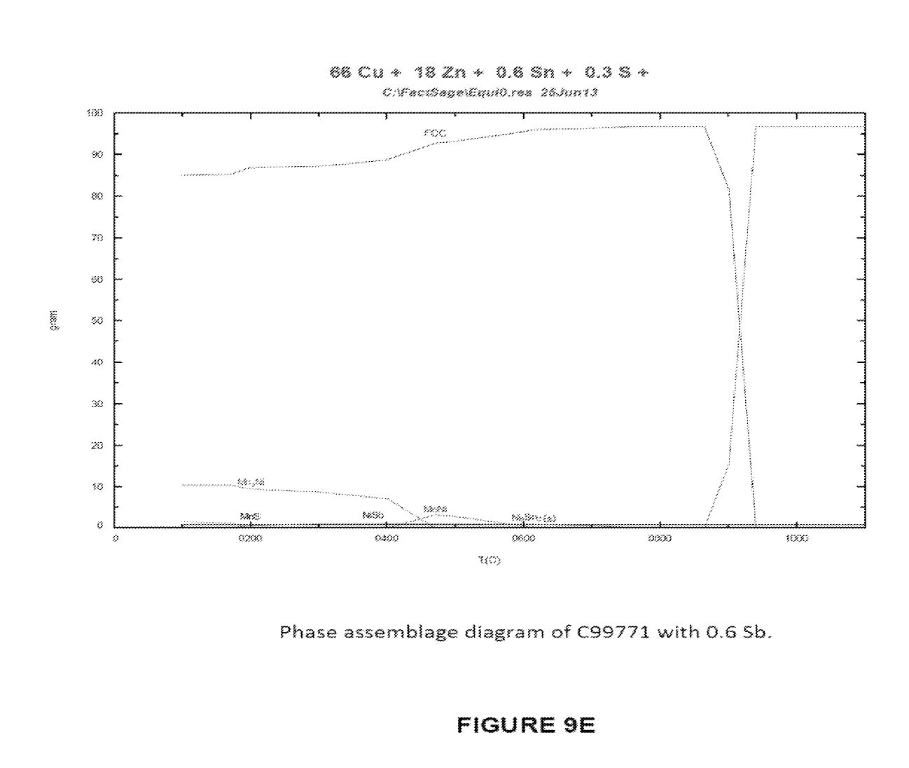

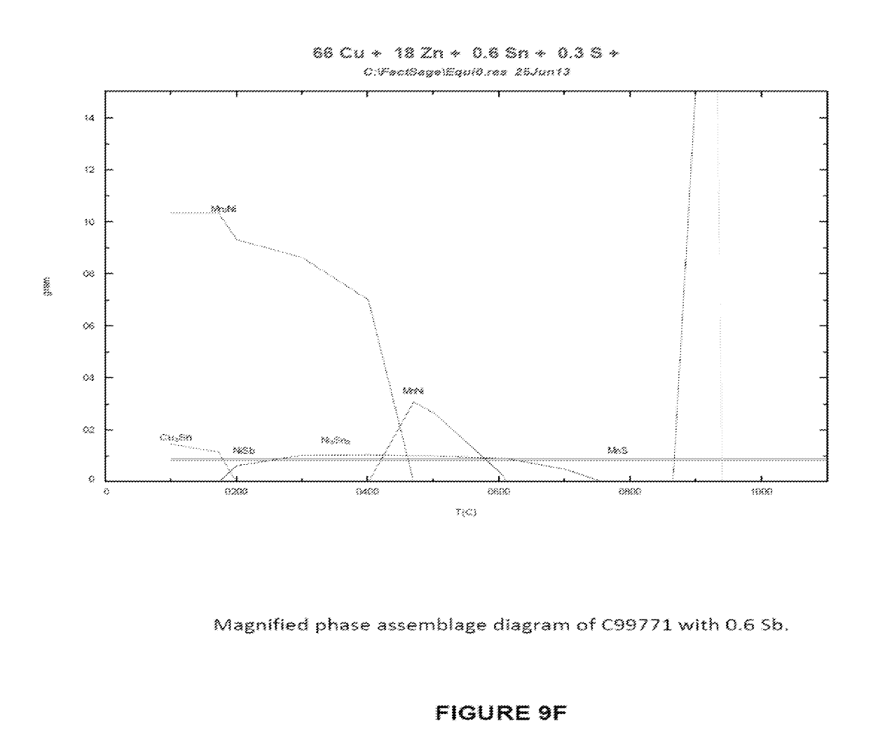

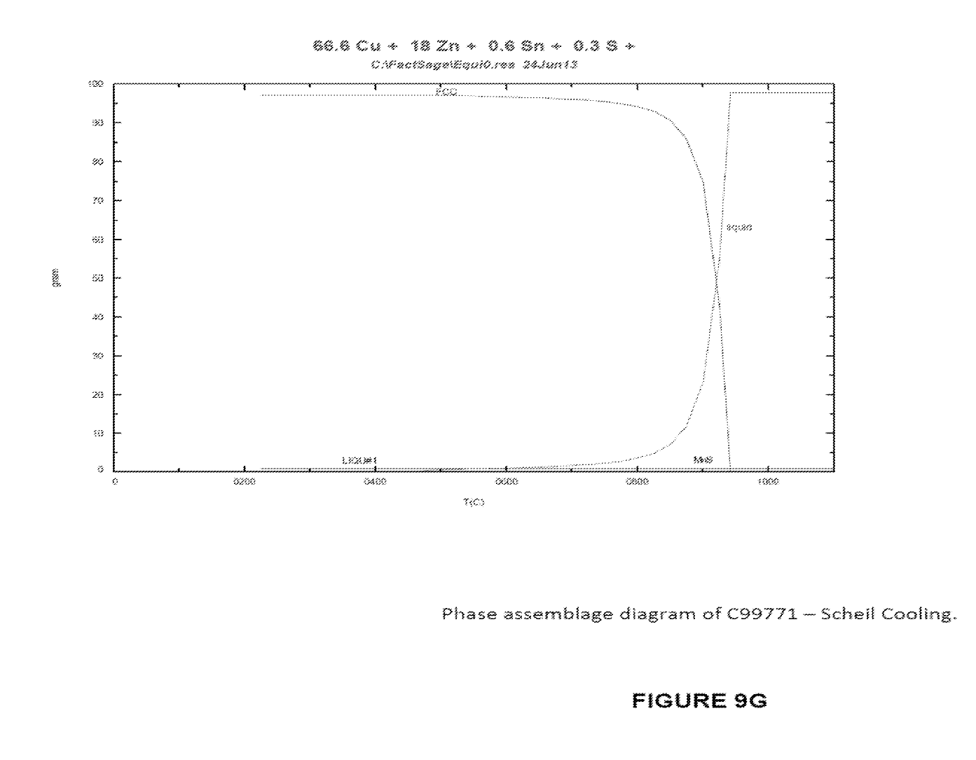

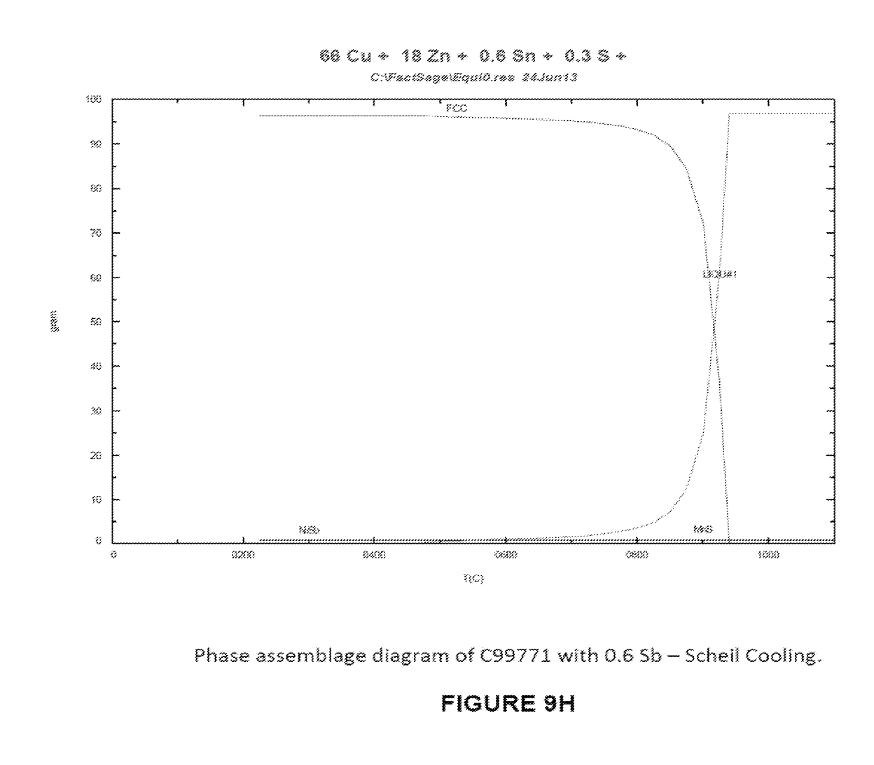

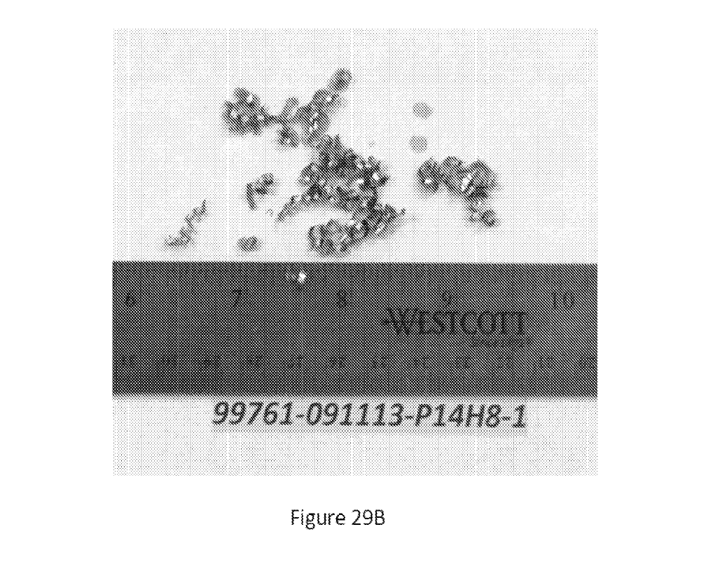

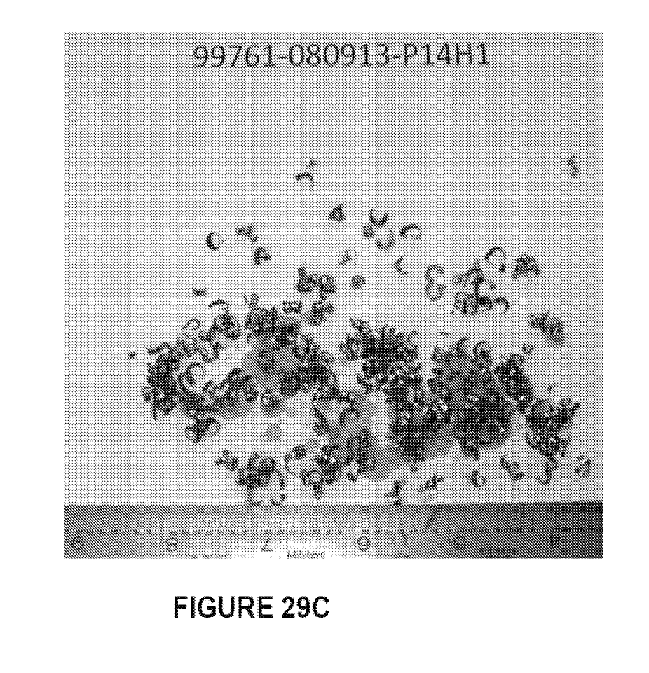

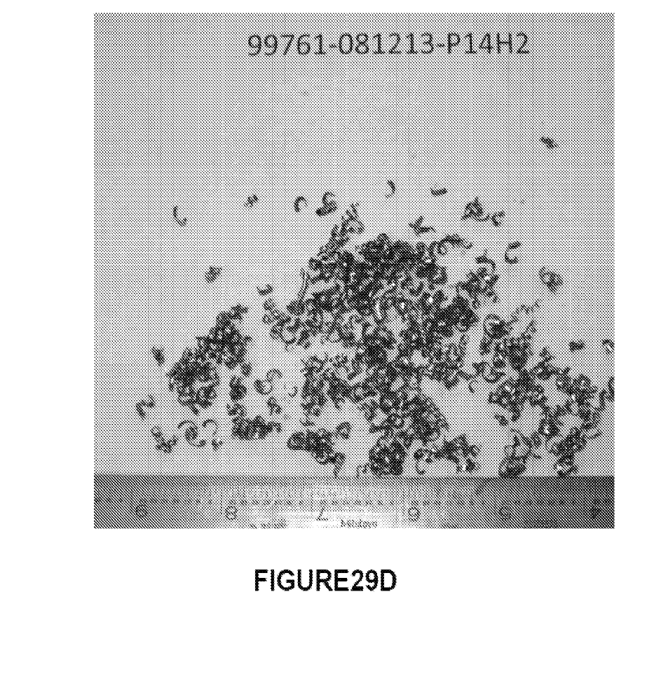

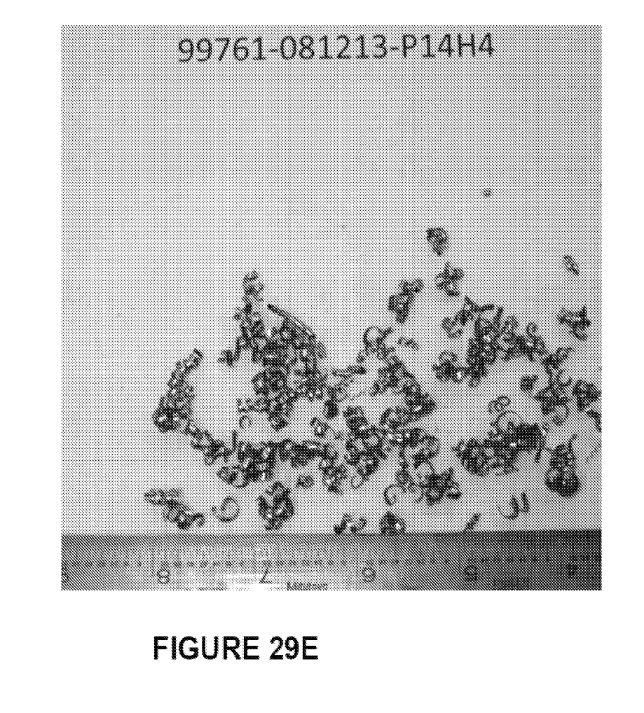

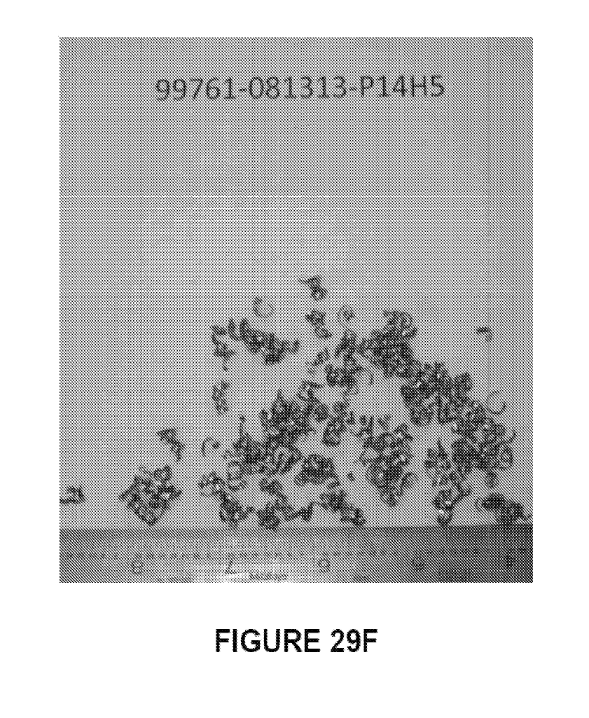

Typically, antimony is picked up from inferior brands of tin, scrap and poor quality of ingots and scrap. For many brass alloys, antimony has been viewed as a contaminant. However, some embodiments of the present application utilize antimony to increase the dezincification resistance, as described further below in regard to the dezincification study. Antimony is used as an alloying element in one embodiment. Phase diagram analysis (FIGS. 8 and 9) shows that Sb forms the NiSb compound. FIGS. 3A-3B show that embodiments having antimony have good mechanical properties and FIGS. 29B-F and 30B-F show good machinability despite the presence of 0.01 to 0.025% S. This is believed to be due to Sb. It is believed that presence of sulfides and NiSb contribute to good machinability. However, it is further believed that as Sb content increases, strength and % elongation decrease.

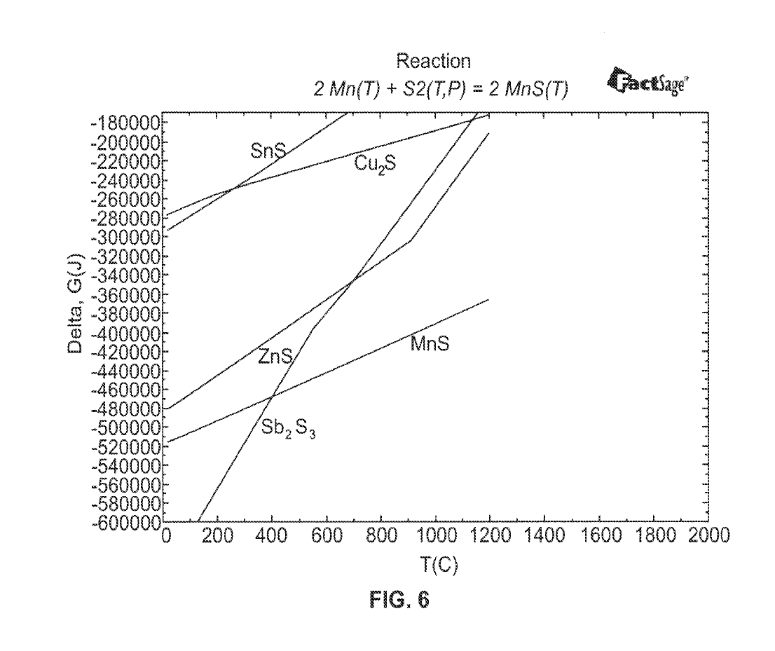

Sulfur is added to the alloys of the present invention to overcome certain disadvantages of using leaded copper alloys. Sulfur provides similar properties as lead would impart to a copper alloy, such as machinability, without the health concerns associated with lead. Sulfur present in the melt will typically react with transition metals also present in the melt to form transition metal sulfides. For example, copper sulfide and zinc sulfide may be formed, or, for embodiments where manganese is present, it can form manganese sulfide. FIG. 6 illustrates a free-energy diagram for several transition metal sulfides that may form in embodiments of the present invention. The melting point for copper is 1,083 Celsius, 1130 Celsius for copper sulfide, 1185 Celsius for zinc sulfide, 1610 Celsius for manganese sulfide, and 832 Celsius for tin sulfide. Thus, without limiting the scope of the invention, in light of the free energy of formation, it is believed that a significant amount of the sulfide formation will be manganese sulfide. It is believed that sulfides solidify after the copper has begun to solidify, thus forming dendrites in the melt. These sulfides aggregate at the interdendritic areas or grain boundaries. The presence of the sulfides provides a break in the metallic structure and a point for the formation of a chip in the grain boundary region and improve machining lubricity, allowing for improved overall machinability. The sulfides predominate in the alloys of the present invention provide lubricity.

Further, good distribution of sulfides improves pressure tightness, as well as, machinability. It is believed that good distribution of the sulfides may be achieved through a combination of hand stirring in gas-fired furnace, induction stirring during induction melting and the plunging of antimony sulfide wrapped in copper foils. Dissociation of antimony sulfide into antimony and sulfur makes it easy for homogeneous formation of copper sulfide and zinc sulfide in comparison with plunging sulfur powder and hence, a homogeneous distribution of the sulfide in interdendritic areas. In one embodiment the sulfur content is below 0.25%. Although sulfur provides beneficial properties as discussed above, increased sulfur content can reduce other desirable properties. It is believed that one mechanism causing such reduction may be the formation of sulfur dioxide during the melt, which leads to gas bubbles in the finished alloy product.

Lead has typically been included as a component in copper alloys, particularly for applications such as plumbing where machinability is an important factor. Lead has a low melting point relative to many other elements common to copper alloys. As such, lead, in a copper alloy, tends to migrate to the interdendritic or grain boundary areas as the melt cools. The presence of lead at interdendritic or grain boundary areas can greatly improve machinability and pressure tightness. However, in recent decades the serious detrimental impacts of lead have made use of lead undesirable in many applications of copper alloys. In particular, the presence of the lead at the interdendritic or grain boundary areas, the feature that is generally accepted to improve machinability, is, in part, responsible for the unwanted ease with which lead can leach from a copper alloy. Alloys of the present invention seek to minimize the amount of lead, for example using less than about 0.09%.

It is believed that the presence of Zn is similar to that of Sn but to a lesser degree, in certain embodiments approximately 2% Zn is roughly equivalent to 1% Sn with respect to the above mentioned improvements to characteristics noted. It is believed that the presence of a high amount of tin increases the strength and hardness but reduces ductility by solid solution strengthening and by forming Cu—Sn intermetallic phase such as Cu₃Sn. It also increases the solidification range. Casting fluidity increases with tin content, and tin also increases corrosion resistance. Tin content of certain embodiments is very low (<1.5%) relative to the prior art. At such low levels, it is believed that Sn remains in solid solution and does not form the Cu₃Sn intermetallic compound. It also does not affect (increase) the solidification range. Such embodiments are long freezing range alloys because of the high Zn, Ni and Mn contents. Cu—Zn binary alloys have short freezing ranges. Cu—Ni binary alloys have a short to medium freezing range depending on the Ni content. Cu—Mn binary alloys have a medium to long freezing range depending on the Mn content. Hence, certain Cu—Zn—Mn—Ni alloys of the present invention will have a long freezing range

With respect to certain alloys, iron can be considered an impurity picked up from stirring rods, skimmers, etc. during melting and pouring operations, or as an impurity in the base ingot. Such categories of impurity have no material effect on alloy properties. However, embodiments of the present invention include iron as an alloying component, preferably in the range of about 0.6%. In certain embodiments iron may be present only as an unintended component in trace amounts.

In some embodiments, nickel is included to increase strength and hardness. On the other hand Ni has a negative zinc equivalent of 1.3. Thus, 10% Ni reduces Zn equivalent by 13%. Generally a higher zinc equivalent is associated with higher strength for an alloy. Other alloying elements such as Al, Sn, Mn have a positive effect on zinc equivalent. Further, nickel aids in distribution of the sulfide particles in the alloy. In one embodiment, adding nickel helps the sulfide precipitate during the cooling process of the casting. The precipitation of the sulfide is desirable as the suspended sulfides act as a substitute to the lead for chip breaking and machining lubricity during the post casting machining operations. Without limiting the scope of the invention, with the lower lead content, it is believed that the sulfide precipitates will minimize the effects of lowered machinability. Further, the addition of nickel, and the ability of the alloy to maintain desirable properties with 2-10% nickel content, provides for an copper alloy that exhibits a color more similar to that of nickel metal rather than copper metal, for example a white to silver color, while not resulting in the increased cost and decreased properties that is associated with higher levels of nickel. Binary Cu—Ni alloys have complete solubility. As the Ni content increases strength increases so also the color of cast components. Generally, three types of cupronickel alloys are commercially available [90/10 (C96200), 80/20 (C96300) and 70/30 (C96400)]. The silver white color increases with Ni content. The cupronickels have very high melting points, 1150-1240 C; but their UTS and YS are also high due to the addition of Nb and Si which form niobium silicide to contribute the strength. Cupronickel alloys typically are cost-prohibitive for many applications. Cupronickels are also harder to machine. Nickel Silver alloys (C97300, C97400 etc) have 11-17% Ni and 17-25% Zn and typically include significant amounts of lead. The nickel silvers contain 8-11% Pb in C97300 and 4.5-5.5% Pb in C97400. They contain very little Mn and hence the melting point is relatively high compared with C99761 and C99771; e.g. 1040 C or 1904 F for C97300 and 1100 C or 2012 F for C97400. Melting points of C99761 and C9971 are 1024 C or 1875 F and 995 C or 1823 F respectively. There are nickel silvers with 27% Ni and less than 4% Zn. Nickel silvers do not contain silver. The silver white color comes from Ni. High nickel content is utilized because lower amounts of nickel results in poor strength properties. In implementations of the present invention, it is believed that the white/silver color comes from Ni and Zn and the presence of zinc in the quantity noted in FIGS. 2A, 3A, 4A, and 5A results in improved strength properties. In general, the higher the amount of Ni, the more silver/white the color approaching the color of elemental nickel.

Phosphorus may be added to provide deoxidation. The addition of phosphorus reduces the gas content in the liquid alloy. Removal of gas generally provides higher quality castings by reducing gas content in the melt and reducing porosity in the finished alloy. However, excess phosphorus can contribute to metal-mold reaction giving rise to low mechanical properties and porous castings. It should be limited to about 0.05% in certain embodiments.

Aluminum in some brass alloys is treated as an impurity. In such embodiments, aluminum has harmful effects on pressure tightness and mechanical properties. However, aluminum in certain casting applications can selectively improve casting fluidity. It is believed that aluminum encourages a fine feathery dendritic structure in such embodiments which allows for easy flow of liquid metal. In certain embodiments aluminum is an alloying element. It increases strength considerably by contributing to the zinc equivalent of the alloy. 1% Al has a zinc equivalent of 6. Preferably, aluminum is included as 2% max.

Silicon is generally considered an impurity. In foundries with multiple alloys, silicon based materials can lead to silicon contamination in non-silicon containing alloys. A small amount of residual silicon can contaminate semi red brass alloys, making production of multiple alloys nearly impossible. In addition, the presence of silicon can reduce the mechanical properties of semi-red brass alloys. For embodiments of the present invention, silicon is not an alloy component and is considered an impurity. It should be limited to below 0.05% and preferably 0.

Manganese may be added in certain embodiments. The manganese is believed to aid in the distribution of sulfides. In particular, the presence of manganese is believed to aid in the formation of and retention of zinc sulfide in the melt. In one embodiment, manganese improves pressure tightness. In one embodiment, manganese is added as MnS. The phase diagrams illustrate that for certain embodiments only 1% MnS forms. Hence, for these embodiments it is believed that MnS is not the predominating sulfide but rather ZnS and Cu₂S will be the predominating sulfides. This is further the result of much of the sulfur being lost to the dross. As FIGS. 8 and 9 illustrate, much of the manganese is present as MnNi₂ (8 wt % in C99761) and Mn₃Ni (˜10 wt % in both C99761 and C99771) due to the higher nickel and manganese levels comparative to certain prior art alloys. In certain embodiments, the Mn content is kept high to reduce the melting point of the alloys.

Manganese serves several important roles. First, by reducing melting point and second, forming intermetallic compounds with Ni. The melting point of binary Cu-11 Mn alloy is reduced by ˜85 C from that of Cu. Similarly, the melting point of Cu-13 Zn is reduced by ˜25 C. By contrast, Ni increases the melting point of the alloy. For the Cu-10 Ni alloy, the increment is about 50 C. When one considers a quaternary alloy of Cu—Ni—Zn—Mn, an overall decrease in melting point can be expected. This expectation has been observed, for example, where the phase diagram indicates a melting point of about 995 C for the 4% Ni alloy (C99771). Hence, embodiments of the present invention can be poured at relatively lower temperatures. This is a significant factor in reducing melt loss and electricity usage (and energy cost). In one embodiment, with about 10% Ni, the melting point is about 1024 C, close to 975 C. This is supported by the phase diagrams in FIG. 8 and the data from differential scanning calorimetry

The second effect of Mn is the formation of intermetallic compounds with Ni which probably contribute to strength and ductility.

A third possible effect of Mn could be its zinc equivalent factor of +0.5. Thus, 11% Mn is equivalent to adding 5.5% Zn. On the other hand Ni has a negative zinc equivalent of 1.3. Thus, 10% Ni reduces Zn equivalent by 13%. For comparison, Zn equivalent of Sn, Fe, and Al are respectively +2, +0.9, and +6. Generally, the higher the Zn equivalent, the higher the strength of the alloy.

The lower nickel content of embodiments of C99761 and C99771 compared with prior art alloys provides for a lower melting point. The presence of relatively larger amounts of zinc, which would normally present a dezincification issue, is overcome with the presence of antimony and other components as described herein.

### Alloy Applications

Both C99761 and C99771 can be utilized for sand casting or permanent mold casting. Advantages of permanent mold casting are a fine grain structure due to faster cooling conditions and better tarnish resistance.

In one implementation, alloys may be used in place of stainless steel. In particular, the alloys may be used in medical applications where stainless steel is used, the alloys provide an antimicrobial functionality. The antimicrobial characteristics of the C99761 and C99771 alloys excel especially in comparison to typical stainless steel. For example, scratches or crevices can form on stainless steel components either during polishing or by rough handling. Micro-organisms can stay there which is not desirable in the many applications.

Embodiments for use as a replacement for stainless steel exhibit a generally higher UTS, YS, and % elongation. In one embodiment, the copper alloy comprises greater than 60% copper, exhibiting antimicrobial effect and a muted copper or white/silver color. However, the stainless steel has a UTS of above about 69 ksi, a YS above about 30 ksi, and a % elongation above about 55%. The minimum requirements for stainless steel are UTS/YS/% Elong of 70 ksi/30 ksi/30. For applications where the improved UTS and YS are required of stainless steel but % elong of stainless steel is not, embodiments utilizing greater than 0.6% aluminum in C99761 or C99771 are used. As can be seen in the data of tables 2B, 3B, 4B, and 5B, increased aluminum is associated with increased UTS and YS at the expense of reduced % elong. For more general applications with moderate mechanical properties such as 40 ksi UTS, 20 ksi YS and 15-20% elongation, Sn and Al ranges can be between 0.5-1.2% and 0.2-1.4% respectively. At high levels of Sn, low Al contents can be used to get the average mechanical properties and vice versa. However, if high UTS and YS (>50 ksi UTS and >30 ksi YS) at the expense of low elongation are desirable for certain applications, high end of the Sn and Al ranges (1-1.5% Sn and 1-2% Al) can be used. In general, for average mechanical properties, Sn+Al content is about 1.5 total wt %. For high strength properties with low elongation, Sn+Al is excess of 2.5 total wt %.

It is further believed that in one implementation, the alloys will have sufficiently higher mechanical properties than prior art alloys to allow for reduced thickness in component casting, thereby offsetting the higher cost of the raw materials. Such alloys are amenable to permanent mold castings despite the long freezing range. The mechanical properties following permanent mold casting are relatively higher (40-62 ksi UTS, 20-35 ksi YS and 7-20% elongation). In addition, section thickness of components can be further reduced in permanent mold casting as a result of improved mechanical properties

### Mechanical Properties

As referenced above, the mechanical properties of the alloy are important to the feasibility for use in different applications. The mechanical properties of C99761 sand cast (FIG. 2A), C99761 permanent mold (FIG. 3A), C99771 sand cast (FIG. 4A), and C99771 permanent mold (FIG. 5A) are set forth respectively in the tables of FIGS. 2B, 3B, 4B, and 5B.

Average sand-cast mechanical properties as reported in FIG. 2B for C99761 are 42 ksi UTS, 29 ksi YS and 13% elongation. As reported in FIG. 3B, the permanent mold cast properties are 47 ksi UTS, 29 ksi YS, and 11% Elong respectively.

Average properties for sand-cast (FIG. 4B) C99771 are 43 ksi UTS, 21 ksi YS, and 24% Elong. For permanent mold cast C99771 (FIG. 5B), the averages are and 51 ksi UTS, 28 ksi YS, and 13% Elong.

Embodiment of the present alloys C99761 and C99771 have a higher content range of tin and aluminum compared to the prior alloys described in related application Ser. No. 14/175,802. One implementation of the present alloys allows for improved UTS and YS at the expense of % Elong. Such alloys allow the reduction in thickness of cast components; especially in permanent mold casting. The results of the mechanical properties are summarized in the tables below. The 761 and 771 versions have relatively low Cu and high Zn. Hence, alloy cost is low.

TABLE 1
White Metal: Comparison of Composition and Mechanical Properties
(Sand Cast)

| Alloy | Cu | Ni | Zn | Mn | UTS | YS | % Elong | Hardness |
|---|---|---|---|---|---|---|---|---|
| C99761 | 58-64 | 8-10 | 16-21 | 8-12 | 42 | 29 | 13 | 87 |
| C99771 | 62-70 | 2-4 | 16-21 | 8-12 | 43 | 21 | 24 | 72 |
| C99760 | 61-67 | 8-12 | 8-14 | 10-16 | 45 | 22 | 35 | 71 |
| C99770 | 66-70 | 3-6 | 8-14 | 10-16 | 44 | 19 | 36 | 66 |
| C84400 | 78-82 | 1.0 | 7-10 | — | 34 | 15 | 26 | 55 |

TABLE 2
White Metal: Comparison of Composition and Mechanical Properties
(PM Cast)*

| Alloy | Cu | Ni | Zn | Mn | UTS | YS | % Elong | Hardness |
|---|---|---|---|---|---|---|---|---|
| C99761 | 58-64 | 8-10 | 16-21 | 8-12 | 48 | 29 | 11 | 92 |
| C99771 | 62-70 | 2-4 | 16-21 | 8-12 | 51 | 28 | 13 | 87 |
| C99760 | 61-67 | 8-12 | 8-14 | 10-16 | 45 | 26 | 13 | 82 |
| C99770 | 66-70 | 3-6 | 8-14 | 10-16 | 44 | 23 | 16 | 71 |

*C84400 alloy is usually not be cast in permanent molds

FIGS. 24A-B, 25A-B, 26A-B, and 27A-B illustrate the impact of the addition of aluminum and tin to the respective alloys. In each of the alloys, as the content of aluminum and tin increase, with the respective limitations set forth on total individual content of aluminum and tin, the UTS and YS increase but the % Elong decreases. As noted above, the alloys may include, in a preferred embodiment an amount of tin and aluminum in total. Permanent mold applications generally require a % Elong of at least 5, for example if one looks at ASTM B806 for copper permanent mold castings, the lowest elongation specified is 5% for a Bi-containing yellow brass. Embodiments of C99761 and C99771 having higher total tin+aluminum content, for example at least 2.5% must still be constrained within the total of 1.5 wt % tin and 2.0 wt % aluminum to ensure the % Elong does not drop below an acceptable level. As can be seen in the figures, the lowest elongation for C99761 and C99771 is 7% and 9% respectively for permanent mold casting.

For sand castings, % elongation exceeding 15% is desirable. C99761 does not meet this criterion. In this case, elongation varied between 4 and 30%, the very low elongation is at high Sn and Al levels (>2.6 Sn+Al) and the desirable elongation (>15%) at levels of 1 to 1.5 Sn+Al contents.

From FIG. 2B it can be seen that total Al+Sn content of less than 2 provides the desired % Elong for sand casting while maximizing other mechanical properties. Preferably, the Al+Sn content is 1 to 1.5% and most preferably 1-1.25%.

From FIG. 3B it can be seen that total Al+Sn content of greater than 2.5 provides the desired % Elong for permanent molding while maximizing other mechanical properties. Most preferably the Al+Sn content is above 3%.

FIG. 4B it can be seen that total Al+Sn content of the tested heats provide the desired % Elong for and casting while maximizing other mechanical properties.

FIG. 5B it can be seen that total Al+Sn content of the tested heats provide the desired % Elong for permanent molding while maximizing other mechanical properties.

### Machinability

Machinability testing described in the present application was performed using the following method. The piece parts were machined by a coolant fed, 2 axis, CNC Turning Center. The cutting tool was a carbide insert. The machinability is based on a ratio of energy that was used during the turning on the above mentioned CNC Turning Center. The calculation formula can be written as follows:

C_{F}=(E₁/E₂)×100

C_{F}=Cutting Force

E₁=Energy used during the turning of a “known” alloy C 36000 (CDA).

E₂=Energy used during the turning of the New Alloy.

Feed rate=0.005 IPR

Spindle Speed=1,500 RPM

Depth of Cut=Radial Depth of Cut=0.038 inches

An electrical meter was used to measure the electrical pull while the cutting tool was under load. This pull was captured via milliamp measurement.

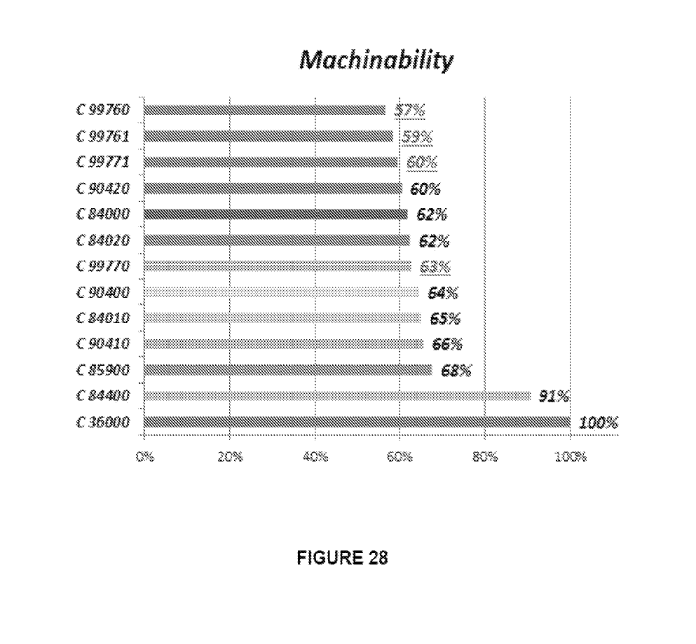

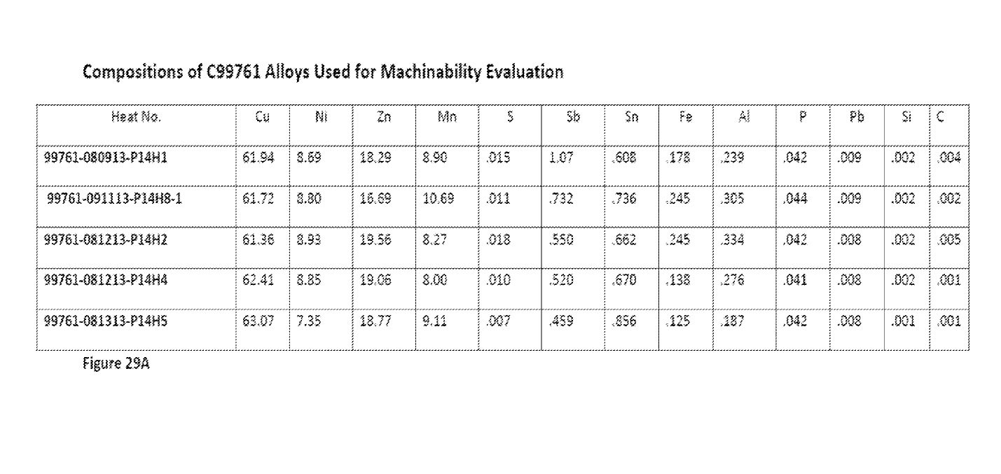

FIG. 28 illustrates a graph comparing machinability of an embodiment of C99761 alloys and an embodiment of C99771 alloys to other known alloys (by CDA registration number). The machinability of the C99761 and the C99771 tested embodiments is comparable to alloys intended for similar uses, including superior performance to the “white” alloy C99760 described in co-pending application Ser. No. 14/175,802.

FIG. 29A lists the compositions of certain heats of a C99761 alloy utilized for machinability evaluations. FIGS. 29B-F illustrates chips from a machinability test of the C99761 heats of FIG. 29A.

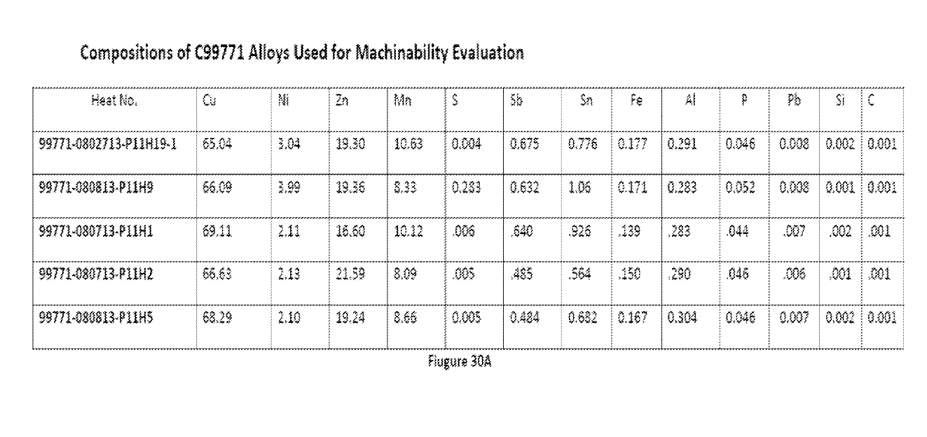

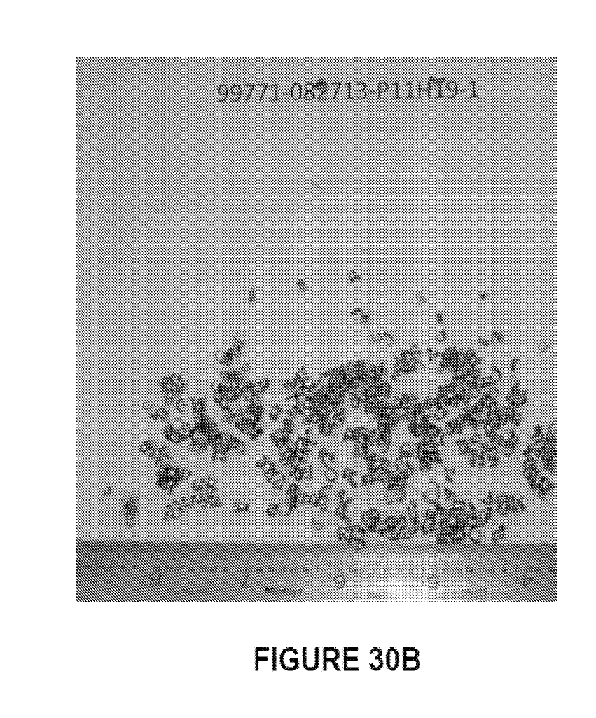

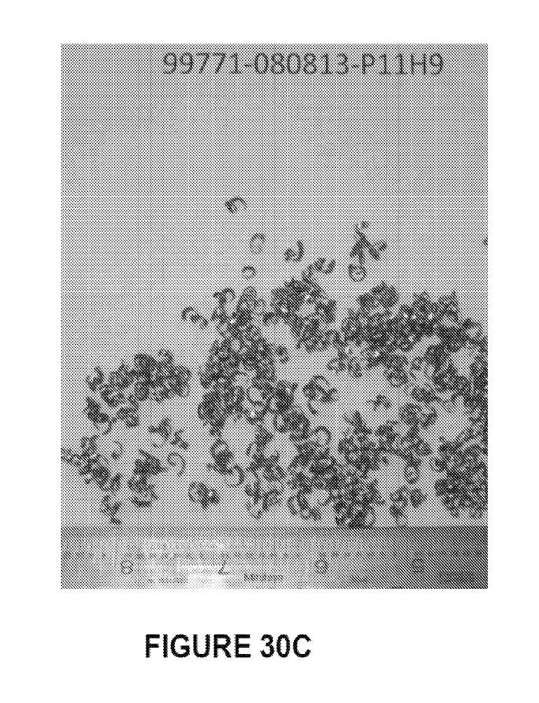

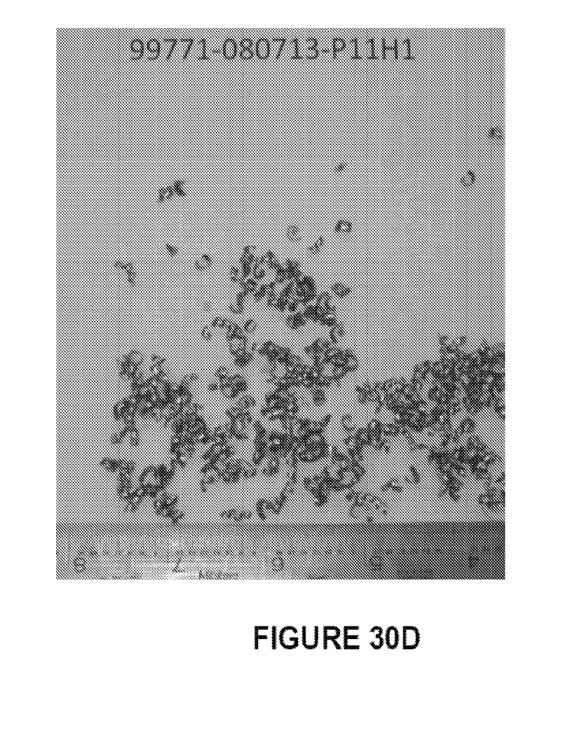

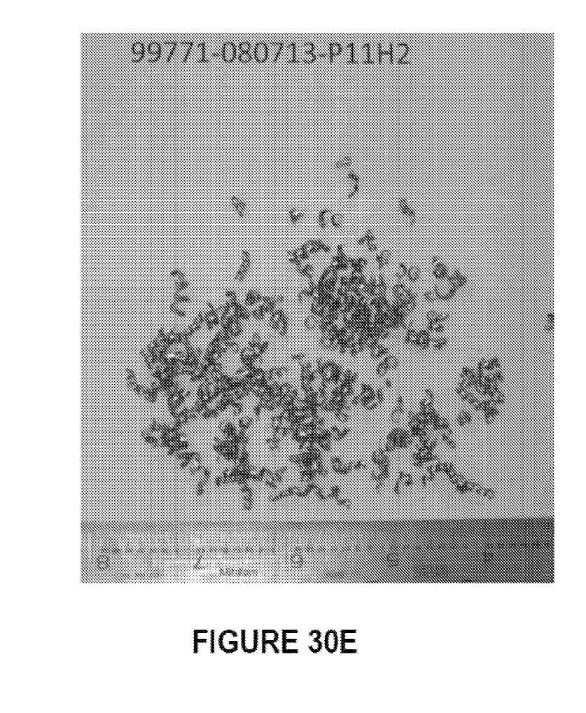

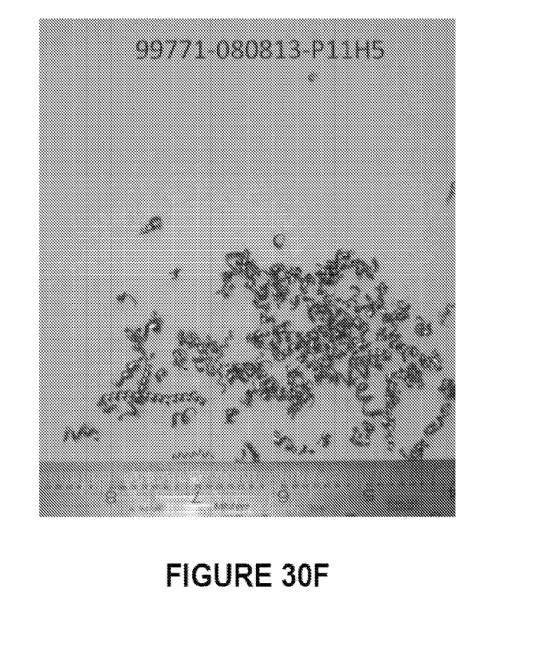

FIG. 30A lists the compositions of certain heats of a C99771 alloy utilized for machinability evaluations. FIGS. 30B-F illustrates chips from a machinability test of the C99771 heats of FIG. 30A.

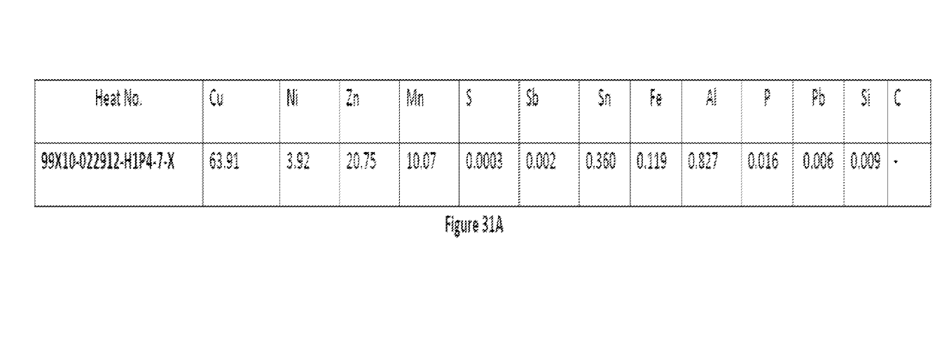

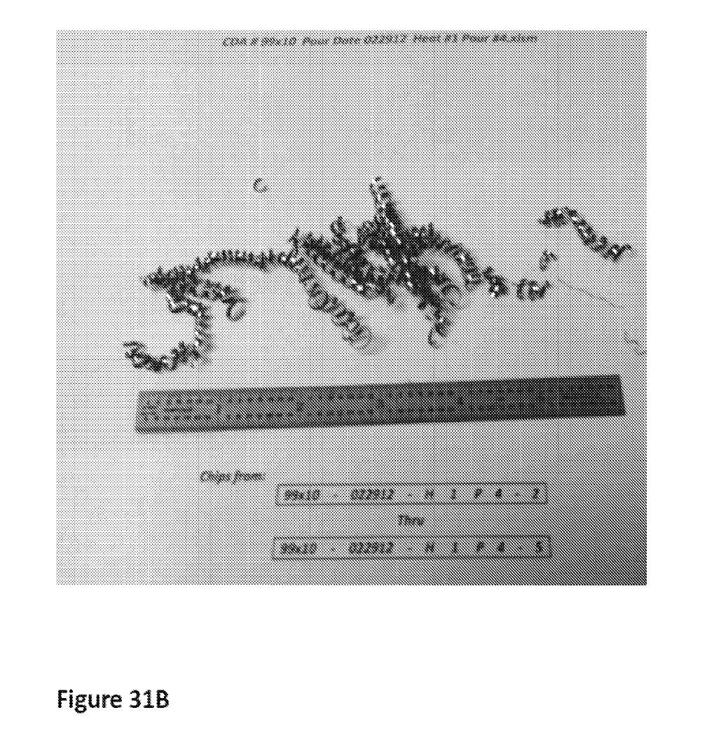

FIGS. 31A-B provide a comparative example of a copper-based alloy free of all but trace antimony and sulfur. As can be seen, both the C99761 embodiment and the C99771 embodiment exhibited good chip morphology as seen in FIGS. 29B-F and 30B-F. The chips exhibit frequent chip-breaking, as explained herein thought to be caused by the sulfide formations and presence of Sb at the interdendritic areas and grain boundaries. In contrast, the alloy set forth in the table of FIG. 31A, without Sb shows in FIG. 31B poor chip formation, with long turnings and infrequent chip breaking. It is believed that the antimony content of the C99771 and C99761 contributes to the improved machinability demonstrated in the chip morphology.

### Phase Diagrams

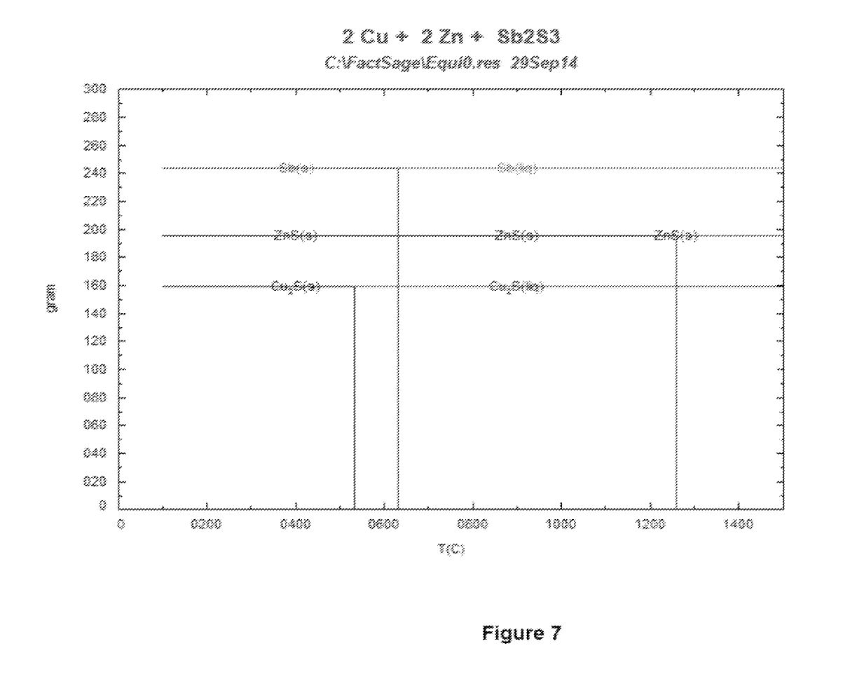

The phases of certain embodiments of the invention have been studied. FIG. 6 is a free energy diagrams of various sulfides. FIG. 7 is a graph of the breakdown of antimony sulfide in molten state. FIGS. 8A-H to 9A-H illustrate corresponding phase diagrams for C99761 and C99771, respectfully.

### Impact of Antimony

FIG. 7 shows the breakdown of antimony sulfide to from antimony and sulfide and the formation of sulfides of other metals. Two moles of antimony sulfide were added in the molten state to one mole of copper and one mole of zinc, both also molten. The antimony sulfide decomposes to provide zinc sulfide at around 1260 Celsius, antimony precipitates at about 630 Celsius, and copper sulfide precipitates at about 520 Celsius.

For one embodiment of the C99761 alloy, a 100 kg overall alloy will contain the following amounts of each phase in kg.

TABLE 3
C99761 Phases

| | Equilibrium | | | | | | | Scheil Cooling | | |
|---|---|---|---|---|---|---|---|---|---|---|
| Composition | FCC | Mn₃Ni | MnNi₂ | Ni₃Sn₂ | NiSb | MnS | Cu₃Sn | FCC | MnS | NiSb |
| C99761 | 80.4 | 9.5 | 8.9 | 1.0 | 0 | 0.3 | 0 | 99.2 | 0.8 | 0 |
| (no Sb) | | | | | | | | | | |
| C99761 | 79.7 | 9.7 | 8.4 | 1.0 | 0.9 | 0.3 | 0 | 98.3 | 0.4 | 0.9 |
| (0.6 Sb) | | | | | | | | | | |

Liquidus and solidus temperatures were determined for both the variation of the C99761 alloy without antimony and an embodiment of C99761 having 0.6% antimony:

TABLE 4
C99761 Liquidus/Solidus

| | Equilibrium | | Scheil Cooling | |
|---|---|---|---|---|
| Composition | Liquidus | Solidus | Liquidus | Solidus |
| C99761 (no Sb) | 980° C. | 895° C. | 980° C. | ~650° C. |
| C99761 (0.6 Sb) | 977° C. | 893° C. | 977° C. | ~650° C. |

For one embodiment of the C99771 alloy, a 100 kg overall alloy will contain the following amounts of each phase in kg.

TABLE 5
C99771 Phases

| | Equilibrium | | | | | | | Scheil Cooling | | |
|---|---|---|---|---|---|---|---|---|---|---|
| Composition | FCC | Mn₃Ni | MnNi₂ | Ni₃Sn₂ | NiSb | MnS | Cu₃Sn | FCC | MnS | NiSb |
| C99771 | 84.5 | 11.4 | 0 | 0 | 0 | 0.8 | 1.4 | 97.2 | 0.8 | 0 |
| (no Sb) | | | | | | | | | | |
| C99771 | 85 | 10.3 | 0 | 0 | 0.9 | 0.8 | 1.4 | 96.4 | 0.8 | 0.9 |
| (0.6 Sb) | | | | | | | | | | |

Liquidus and solidus temperatures were determined for both the variation of the C99771 alloy without antimony and an embodiment of C99771 having 0.6% antimony:

TABLE 6
C99771 Liquidus/Solidus

| | Equilibrium | | Scheil Cooling | |
|---|---|---|---|---|
| Composition | Liquidus | Solidus | Liquidus | Solidus |
| C99771 | 943° C. | 868° C. | 943° C. | ~600° C. |
| +0.6 wt % Sb | 940° C. | 865° C. | 940° C. | ~600° C. |

The phase diagrams have been drawn for both equilibrium and non-equilibrium (Scheil calculation) conditions for both an embodiments of C99761 compared to a variation on C99761 alloys lacking antimony (FIGS. 8A-H) and C99771 compared to a variation on C99771 alloys lacking antimony (FIGS. 9A-H). The embodiment evaluated has a composition for alloy C99761 was 61 Cu, 18 Zn, 9 Ni, 10 Mn, 0.6 Sb, 0.1 S, 0.6 Sn, 0.4 Al, 0.2 Fe and for alloy C99771: 66 Cu, 3 Ni, 18 Zn, 10 Mn, 0.6 Sb, 0.3 S, 0.6 Sn and 0.5 Al. The effect of 0.6% Sb addition is also shown.

It is evident that these are medium freezing range alloys compared with semi-red brass family. For certain embodiments of the present invention, the freezing range is around 75-85 C. For the semi-red brass family, freezing range is greater than 80 C. Thus, permanent mold casting of these embodiments of the present invention are favorable and test bars and tail castings have been successfully cast in both alloys. In some applications, most of the plumbing parts are produced by both gravity and low pressure permanent mold casting. Finer grain structure due to faster cooling rates have increased the mechanical properties in permanent mold casting.

Further liquidus experiments were conducted on the Setaram SetSys2400 DSC to evaluate the solidus and liquidus temperature of the alloys in the table below.

TABLE 7
Samples for Liquidus and Solidus Study

| Alloy | Cu | Ni | Zn | Mn | S | Sb | Sn | Fe | Al | P | Pb |
|---|---|---|---|---|---|---|---|---|---|---|---|
| 99761- | 61.36 | 8.93 | 19.56 | 8.27 | 0.018 | 0.550 | 0.662 | 0.245 | 0.334 | 0.042 | 0.008 |
| 081213- | | | | | | | | | | | |
| P4H2-8 | | | | | | | | | | | |
| 99771- | 69.20 | 3.21 | 17.56 | 8.10 | 0.014 | 0.685 | 0.616 | 0.319 | 0.224 | 0.050 | 0.006 |
| 062713- | | | | | | | | | | | |
| P12H3-9 | | | | | | | | | | | |

To find out the solidus and liquidus temperature the samples were heated from room temperature up to 1100 C, cooled to 800 C, heated to 1100 C a second time, and cooled to 800 C again. Finally the apparatus was brought down to room temperature. These experiments were conducted under an Argon atmosphere which was preceded by vacuum pump evacuation of the DSC chamber. Thus data from two cycles were collected. The heating was done at 10 C/min and the cooling at 15 C/min. The solidus and the liquidus temperatures, obtained from both cycles are provided in the table below.

TABLE 8
Liquidus and Solidus Temperatures

| | 1^{st} Cycle | 1^{st} Cycle | 1^{st} Cycle | 2^{nd} Cycle | 2^{nd} Cycle | 2^{nd} Cycle |
|---|---|---|---|---|---|---|
| | T_{L,} ° C. | T_{S} ° C. | Range, | T_{L} ° C. | T_{S} ° C. | Range, |
| Alloy | (° F.) | (° F.) | ° C. (° F.) | (° F.) | (° F.) | ° C. (° F.) |
| 99760- | 1025 | 939 | 86 | 1020 | 897 | 123 |
| 020613- | (1877) | (1722) | (155) | (1868) | (1647) | (221) |
| P2H1 | | | | | | |
| 99770- | 966 | 843 | 123 | 1025 | 939 | 86 |
| 052313- | (1771) | (1550) | (221) | (1877) | (1722) | (155) |
| P7H1 | | | | | | |
| 99761- | 1024 | 842 | 182 | 1035 | 904 | 131 |
| 081213- | (1875) | (1548) | (327) | (1895) | (1659) | (236) |
| P14H2 | | | | | | |
| 99771- | 995 | 852 | 143 | 997 | 904 | 93 |
| 062713- | (1823) | (1566) | (257) | (1827) | (1659) | (168) |
| P12H3 | | | | | | |

The samples were weighed before and after these experiments. The percent loss in weight was as follows:
Alloy 99761: 20.2%
Alloy 99771: 18.8%
   This might explain the shift of the solidus and liquidus in the first and second cycles.
   The data from the first cycle is more representative of the alloys.

### Zinc Equivalent

Copper alloys are known to undergo dezincification in certain environments when the alloy contains greater than about 15%. However, large amounts of zinc can alter the phase of the copper from an all alpha to a duplex or beta phase. Other elements are known to also alter the phase of the copper. A composite “zinc equivalent” is used to estimate the impact on the copper phase:

Zn_{equivalent}=(100*X)/((X+Cu %)

Where x is the total of zinc equivalents contributed by the added alloying elements plus the percentage of actual zinc present in the alloy. A zinc equivalent under 32.5% Zn typically results in single alpha phase. This phase is relatively soft in comparison with the beta phase.

Zinc Equivalent values were calculated for the C99761 and C99771 formulations shown in the below table, generally both are mid-range compositions of the ranges in the respective FIGS. 2A and 4A. Zinc equivalent was calculated using the above formula given in

TABLE 9
Zinc Equivalence Testing Composition

| Alloying | | | | | | | | |
|---|---|---|---|---|---|---|---|---|
| Element | Cu | Sn | Zn | Ni | Mn | Fe | Sb | Al |
| C99761 | 59.95 | 0.85 | 18.5 | 9.0 | 10 | 0.2 | 0.5 | 1.0 |
| C99771 | 65.95 | 0.85 | 18.5 | 3.0 | 10 | 0.2 | 0.5 | 1.0 |

ZE values for these compositions were found to be 25.6% (C99761) and 29.6% respectively (C99771). ZE for C99771 being higher by 4% over C99761 should exhibit slightly better mechanical properties. This is consistent with the observed mechanical values, particular for the PM casting embodiments. This is also what we have observed (see data on pages 23 and 24), at least for PM casting.

Table 2 lists equivalent zinc values for certain alloying elements described herein. As can be seen, not all elements contribute equally to zinc equivalent. In fact, certain elements, such as nickel have a negative zinc value, thus reducing the zinc equivalent number and the associated mechanical properties with higher levels.

TABLE 10
Zinc Equivalents

| Alloying | | | | | | | | |
|---|---|---|---|---|---|---|---|---|
| Element | Si | Al | Sn | Mg | Pb | Fe | Mn | Ni |
| Zinc | 10 | 6 | 2 | 2 | 1 | 0.9 | 0.5 | −1.2 |
| Equiv. | | | | | | | | |

### Dezincification

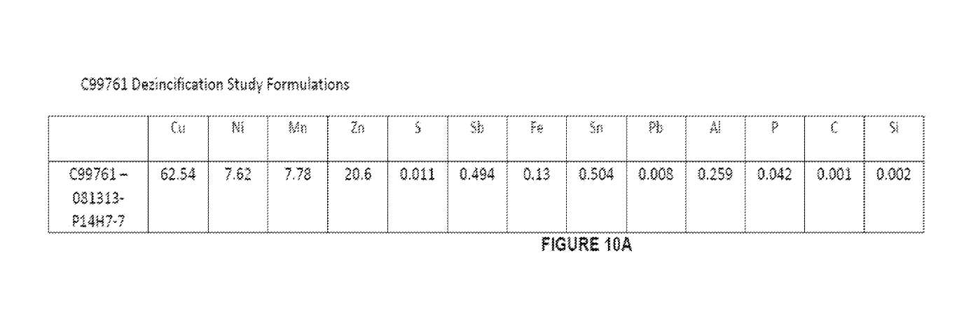

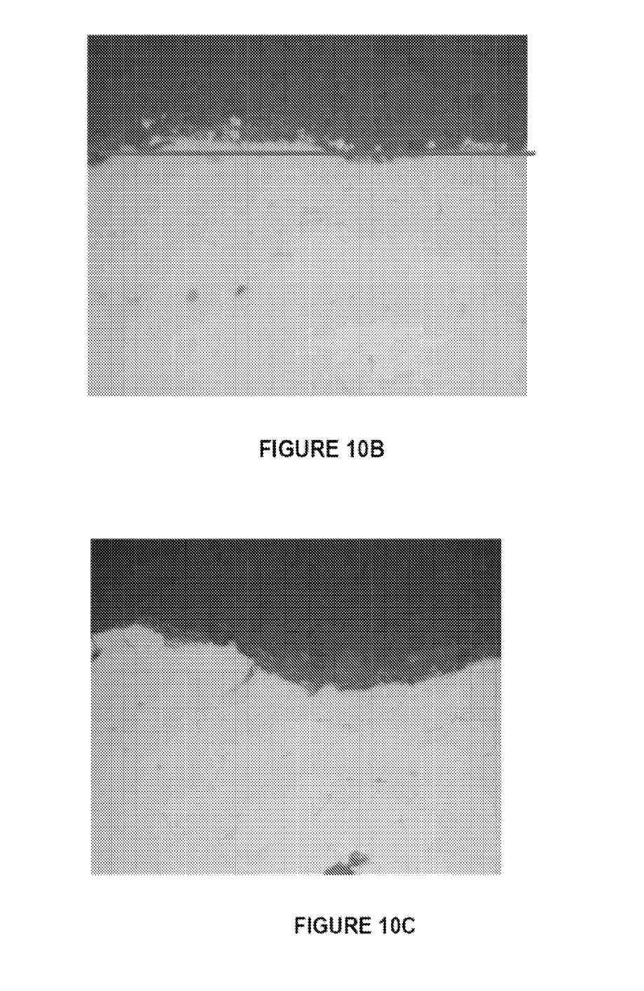

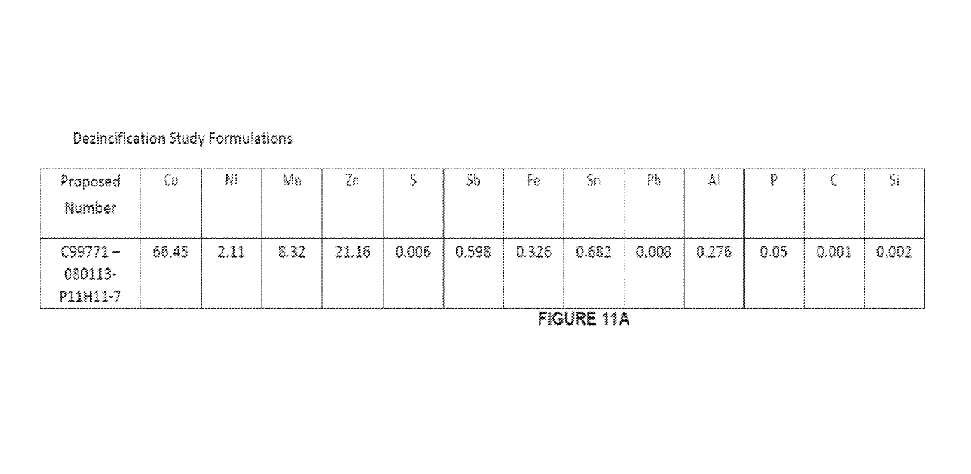

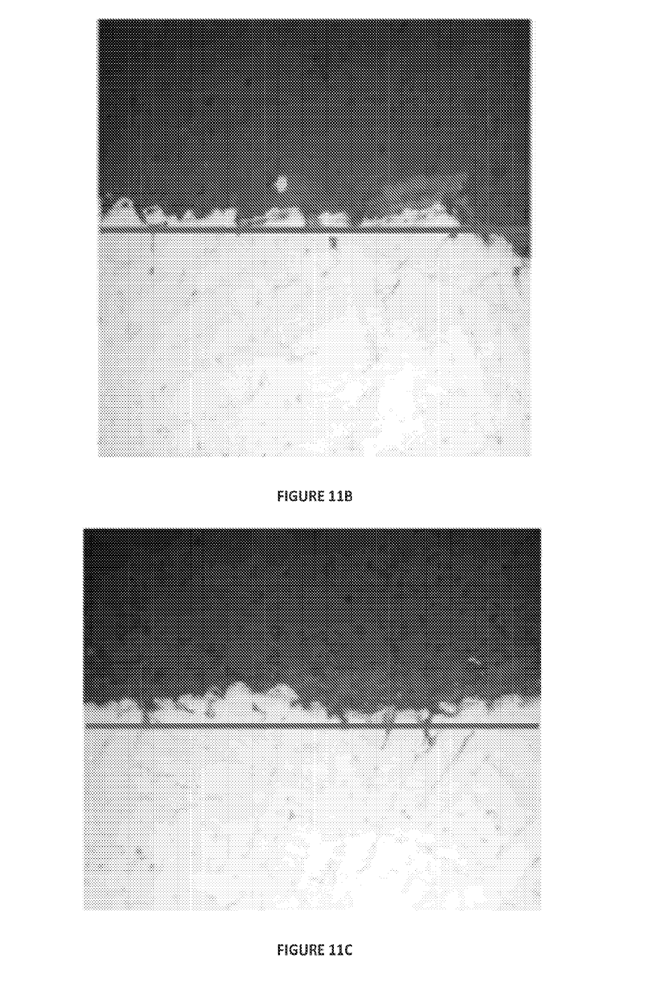

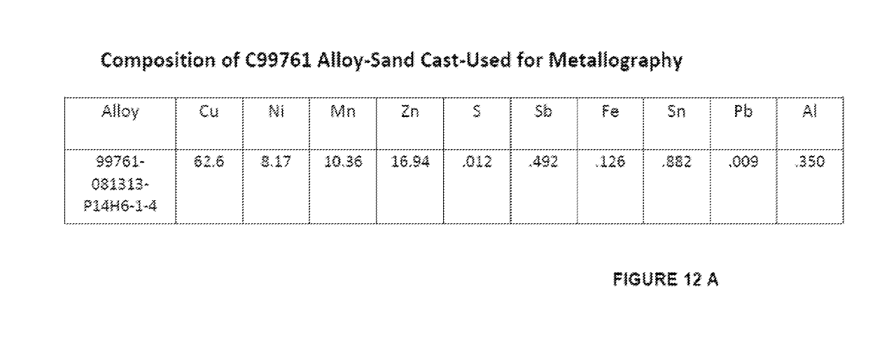

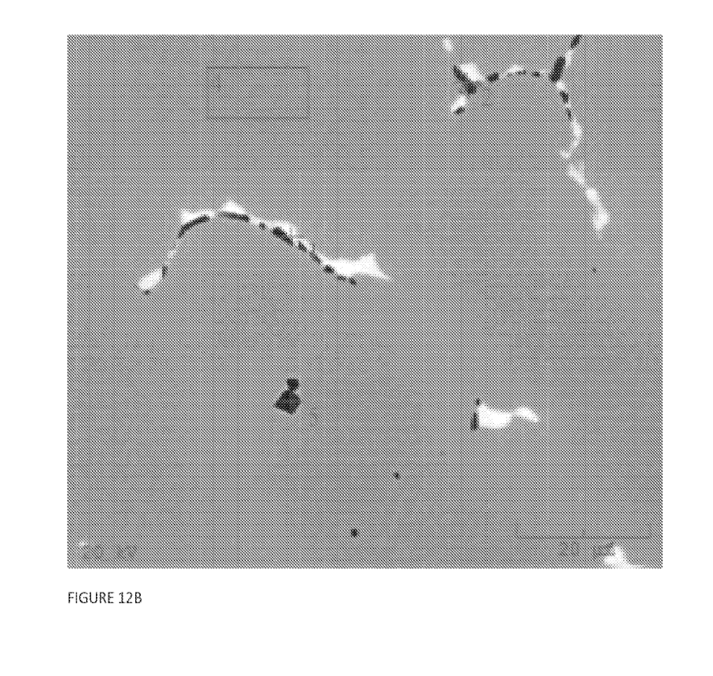

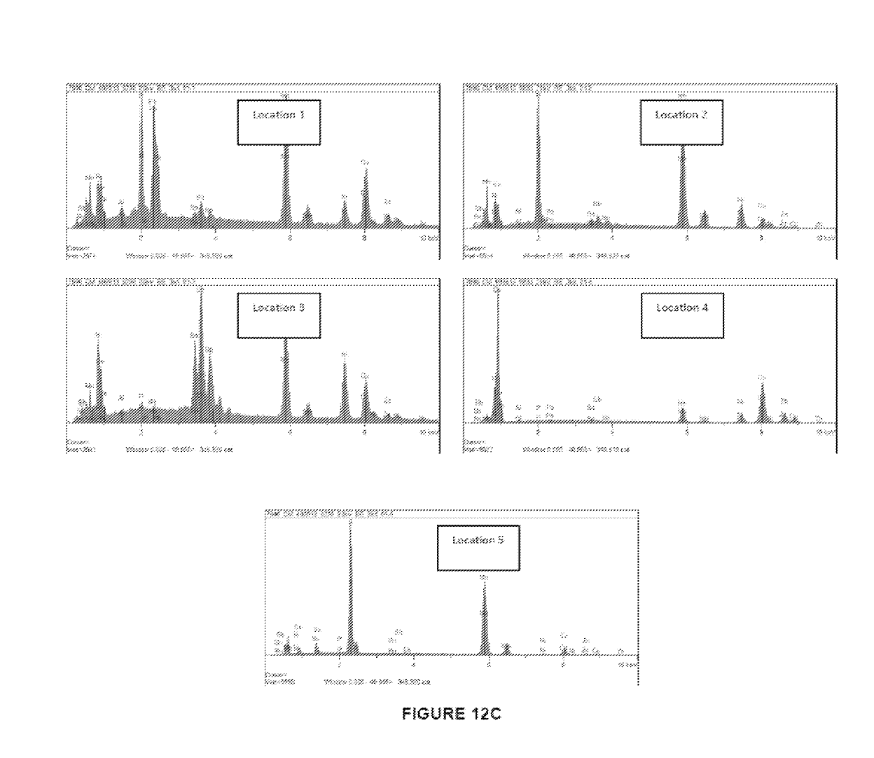

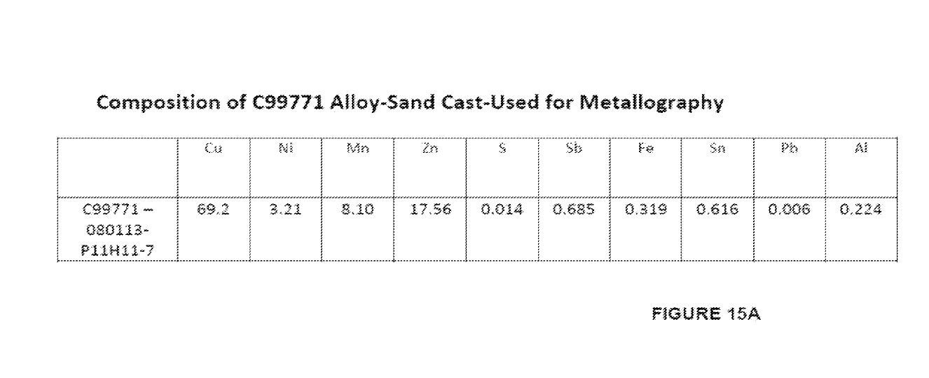

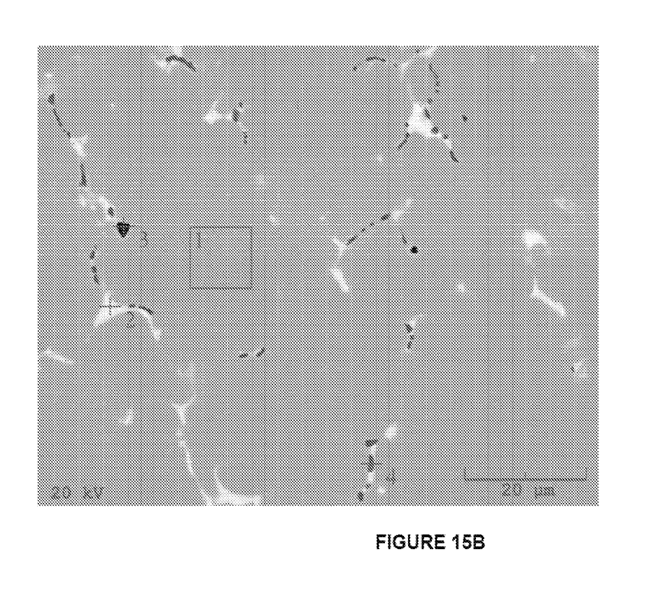

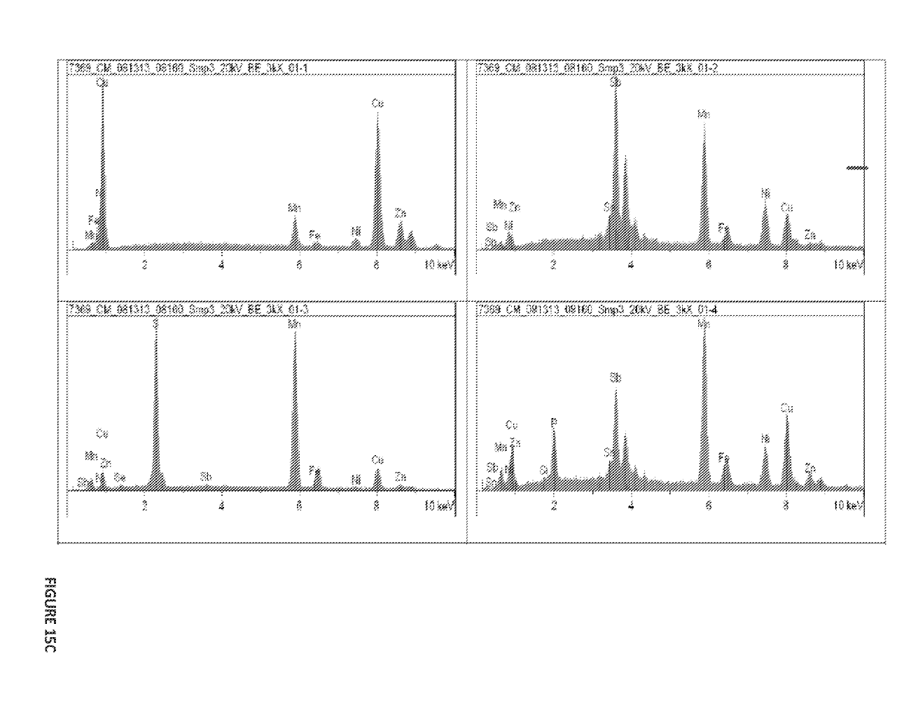

With respect to the information in FIGS. 10A-C and 11A-C a dezincification study was done. The C99761 and C99771 alloy compositions include a higher amount of zinc than would be expected to be a viable while still exhibiting good resistance to dezincification. The surprising performance allows for a lower amount of copper or other components that raise the expense without markedly improving the alloy over the use of zinc. For example, in comparison to the C99760 and C99770 alloys disclosed in copending application Ser. No. 14/175,802, the alloys of the present invention provide for a lower range of copper (offset by a higher range of zinc) without the deleterious effects of dezincification that would be expected from such. FIGS. 10A and 11A list the formulation for the tested alloy. It has been observed that the first series of alloys tested (C99761 in FIG. 2A) with about 8% Ni is less whiter than the second series of alloys tested (C99771 in FIG. 4A) with 2% Ni. Dezincification occurs as Zn, typically when present in excess of 15%, leaches out selectively in chlorinated water. Zinc's reactivity is high because of a weak atomic bond. The Zn—Sb phase diagrams indicate that Sb can form an intermetallic compound such as Sb₃Zn₄ which increases Zn's atomic bond strength. It believed that the reduction of Cu⁺⁺ in solution to Cu on the yellow brass surface is the cathodic reaction accompanying the anodic dezincification reaction. Sb addition inhibits or “poisons” the cathodic reduction reaction and thereby efficiently eliminates dezincification. Thus, it is believed that the increased atomic bond strength increases resistance to selective leaching such that dezincification is minimized. The EDS analysis of C99761 (locations 1 & 3) and C99771 (location 4) described herein further supports this. FIGS. 12B-C (C99761) and FIGS. 15B-C (C99771) show the presence of Zn and Sb in addition to Cu, Ni, and Mn.

### C99761

As shown below there is no dezincification despite high Zn content up to 20.6%. This is due to the presence of Sb. The formulation for the tested C99761 alloy is show in FIG. 10A.

In this test, ground cross sections are immersed in a 1% copper chloride solution at 75±5° C. for 24 hours. At the end of this immersion period, polished cross sections are prepared perpendicular to the exposed surfaces, and the depth of any dezincification corrosion is measured. This analysis was performed in both a thin area and a thick area of the casting per the ISO specification.

The exposed surface in the thin section as shown in FIG. 10B. No dezincification corrosion is evident in the thick section (FIG. 10C). ISO 6509 does not contain any acceptance criteria for the permissible amount of dezincification, however, these depths do not exceed the 100 microns maximum specified in the similar Australian Standard AS 2345, “Dezincification Resistance of Copper Alloys.” The results indicate that the sample is minimally susceptible to dezincification corrosion.

### C99771

As shown below there is no dezincification despite high Zn content up to 21%. This is due to the presence of Sb. The formulation for the tested C99771 alloy is show in FIG. 11A.

In this test, ground cross sections are immersed in a 1% copper chloride solution at 75±5° C. for 24 hours. At the end of this immersion period, polished cross sections are prepared perpendicular to the exposed surfaces, and the depth of any dezincification corrosion is measured. This analysis was performed in both a thin area and a thick area of the casting per the ISO specification. The submitted section exhibited a uniform cross section and, therefore, the two samples were prepared through typical areas on transverse (FIG. 11B) and longitudinal planes (FIG. 11C).

Dezincification corrosion extends from the exposed surface in the sections prepared in the transverse and longitudinal orientations of the submitted sample, as shown in FIGS. 11B and 11C. The corrosion extends to a maximum depth of 0.0002″ (5.1 microns) in the planes of both metallographic sections. ISO 6509 does not contain any acceptance criteria for the permissible amount of dezincification, however, these depths do not exceed the 100 microns maximum specified in the similar Australian Standard AS 2345, “Dezincification Resistance of Copper Alloys.”

This investigation indicates that the submitted sample exhibits slight dezincification corrosion when tested in accordance with ISO 6509, “Corrosion of Metals and Alloys—Determination of the Dezincification Resistance of Brass.” ISO 6509 does not include any acceptance criteria, however, the dezincification depth of this sample does not exceed the 100 micron maximum dezincification depth included in the similar Australian Standard AS 2345, “Dezincification Resistance of Copper Alloys.” These results indicate that this sample is minimally susceptible to dezincification corrosion. By comparison CDA alloy C85400 with 65-67 Cu, 0.5-1.5 Sn, 1.5-3.8 Pb, 24-32 Zn, 1 Ni, 0.35 Al, and 0.05 Si exhibits a depth of dezincification varied between 335 and 1151 microns in the thick areas. Similarly for alloy equivalent to C99780 with 62-66 Cu, 0.3-1.0 Al, 0.5-2.0 Sn, 16-22 Zn, 12-15 Mn, 0.5-2.0 Bi, 4-6 Ni, depth of dezincification was 332-932 microns in thick areas.

### Metallography

### C99761

### Sand Cast

A sample of an embodiment of a C99761 sand-cast alloy sample, having the composition listed in FIG. 12A, was sectioned, mounted in conductive epoxy, and metallographically prepared to a 0.04 micron finish. The tested alloy had a formulation of 62.6 Cu, 8.17 Ni, 16.94 Zn, 10.36 Mn, 0.012 S, 0.492 Sb, 0.882 Sn, 0.126 fe, 0.350 Al, 0.040 P, 0.009 Pb, 0.002 Si, 0.002 C. The sample was examined using a scanning electron microscope with energy dispersive spectroscopy (SEM/EDS). This instrument is equipped with a light element detector capable of detecting carbon and elements with a higher atomic number (i.e., cannot detect hydrogen, helium, lithium, and beryllium, and boron detection is marginal). Images were acquired using the secondary electron (SE) and backscattered electron (BE) detectors. In backscattered electron imaging, elements with a higher atomic number appear brighter. The sample was examined using a 20 kV accelerating voltage.

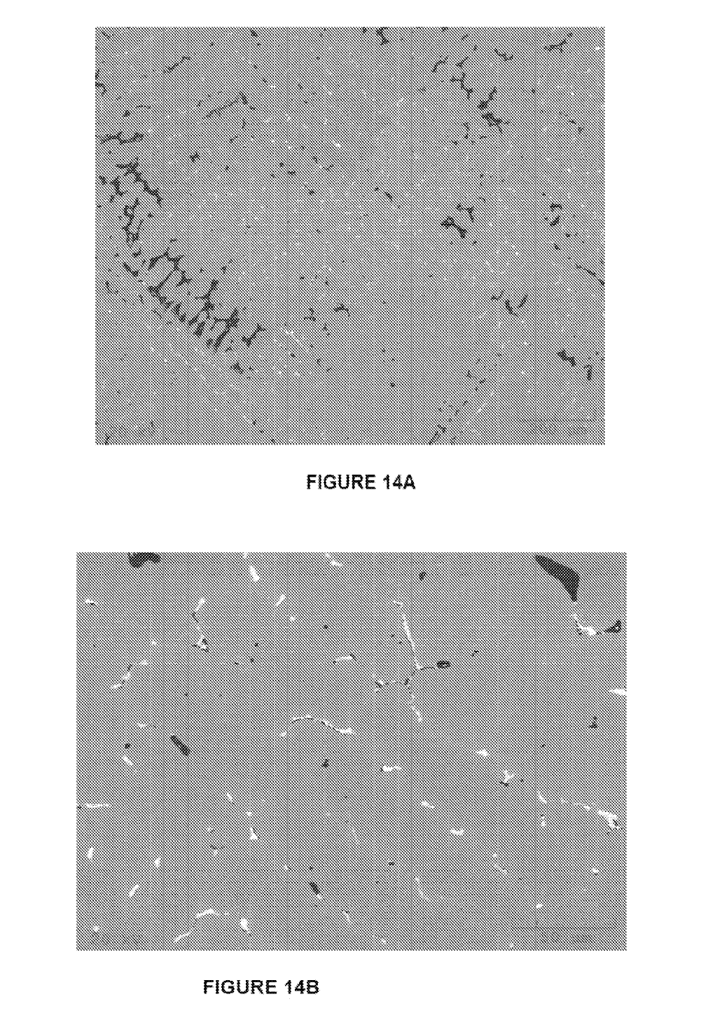

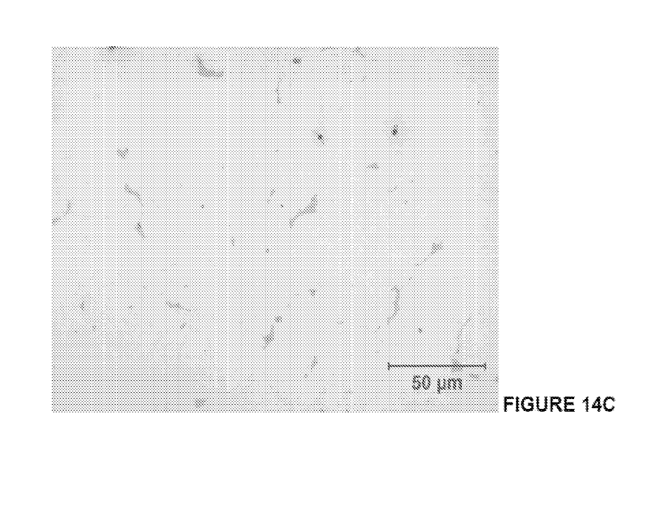

Representative BE images of the microstructure taken at 200X and 1000X are shown in FIGS. 14A and 14B, respectfully. BE imaging with EDS was performed to determine the chemistry of the various secondary phases present in the copper alloy.

FIG. 12B illustrates a BE image of an embodiment of C99761 alloy that is further analyzed at 5 discreet locations via SEM/EDS spectra. The SEM/EDS spectra results of the base material from location 4 consist of high concentrations of copper with lesser amounts of manganese, nickel, and zinc (see Location 4 FIG. 12B). The bright white colored phase reveals high concentrations of lead, phosphorus, and manganese with lesser amounts of copper, nickel, zinc, tin, and antimony (see Location 1, FIG. 12B). This alloy contains only 0.009% Pb. The high concentration of Pb at Location 1 indicates the entrapment of a lead particle. The dark colored phase reveals high concentrations of phosphorus and manganese with lesser amounts of nickel, copper, zinc, tin, and antimony (see Location 2 FIG. 12B). The lighter phase at location 3 reveals high concentrations of tin, antimony, and manganese with lesser amounts of nickel, copper, and zinc (see Location 3, FIG. 12B). The dark colored phase at Location 5 reveals high concentrations of sulfur and manganese with lesser amounts of nickel, copper, zinc, and selenium (see Location 5, FIG. 12B). Semi-quantitative chemical analysis data is reported in the following table for the above locations.

TABLE 11
C99761 Sand Cast EDS Spectra analysis.

| Spectrum | Al | Si | P | S | Mn | Ni | Cu | Zn | Se | Sn | Sb | Pb |
|---|---|---|---|---|---|---|---|---|---|---|---|---|
| Location 1 | <1 | <1 | 8.3 | 0 | 23.1 | 7.0 | 22.3 | 5.0 | 0 | 1.9 | 4.5 | 26.7 |
| Location 2 | 0 | <1 | 19.4 | 0 | 49.0 | 16.1 | 7.4 | 1.4 | 0 | 2.8 | 3.4 | 0 |
| Location 3 | <1 | 0 | <1 | 0 | 19.7 | 17.5 | 14 | 2.5 | 0 | 17.9 | 26.7 | <1 |
| Location 4-Base | <1 | 0 | 0 | 0 | 8.9 | 8.8 | 64.2 | 16 | 0 | 0 | 0 | 0 |
| Location 5 | 0 | 0 | 0 | 31.2 | 49.7 | 1.4 | 9.7 | 2.5 | 4.9 | 0 | 0 | 0 |

Results in weight percent unless otherwise indicated.

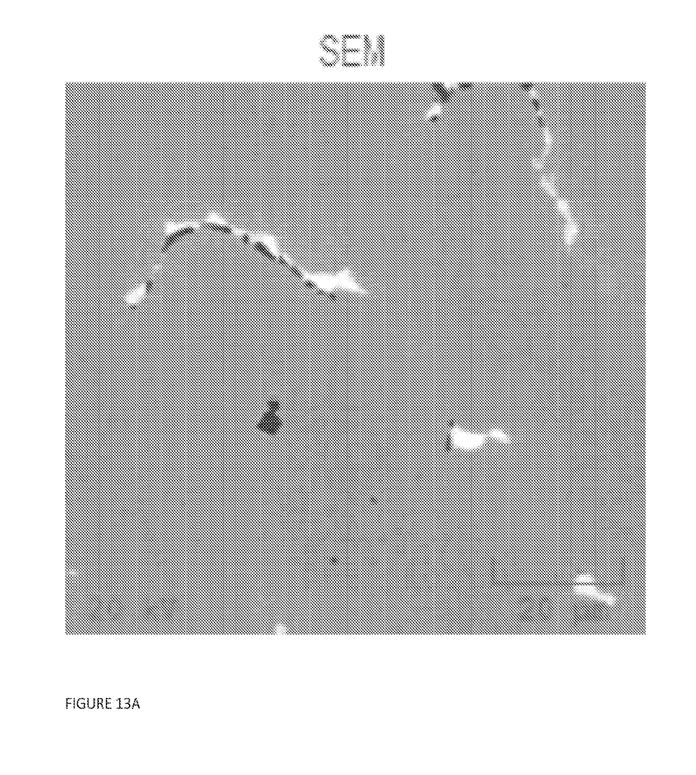

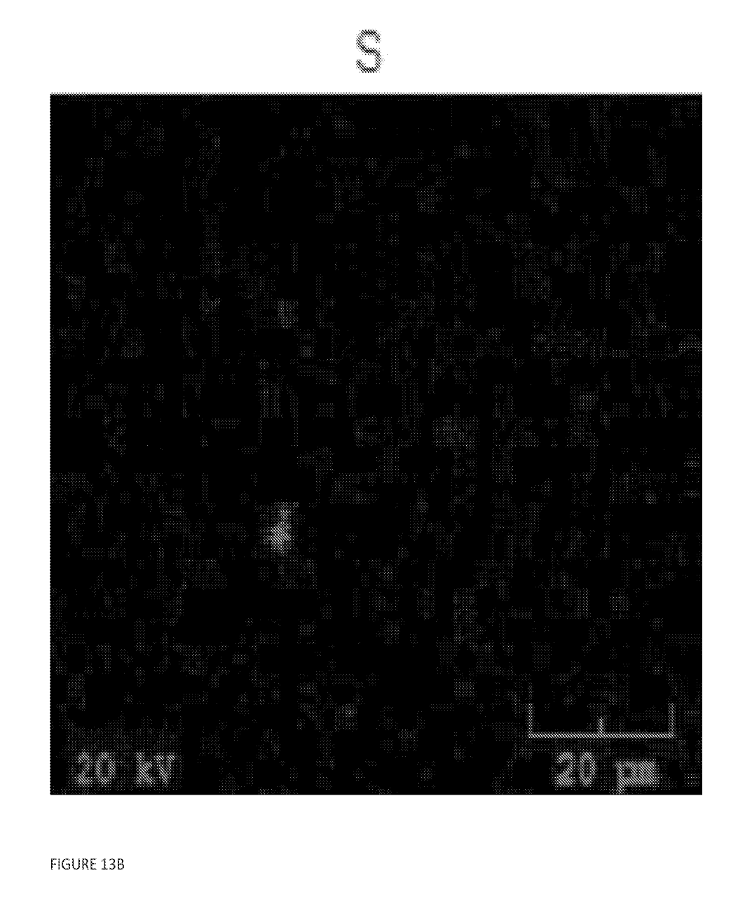

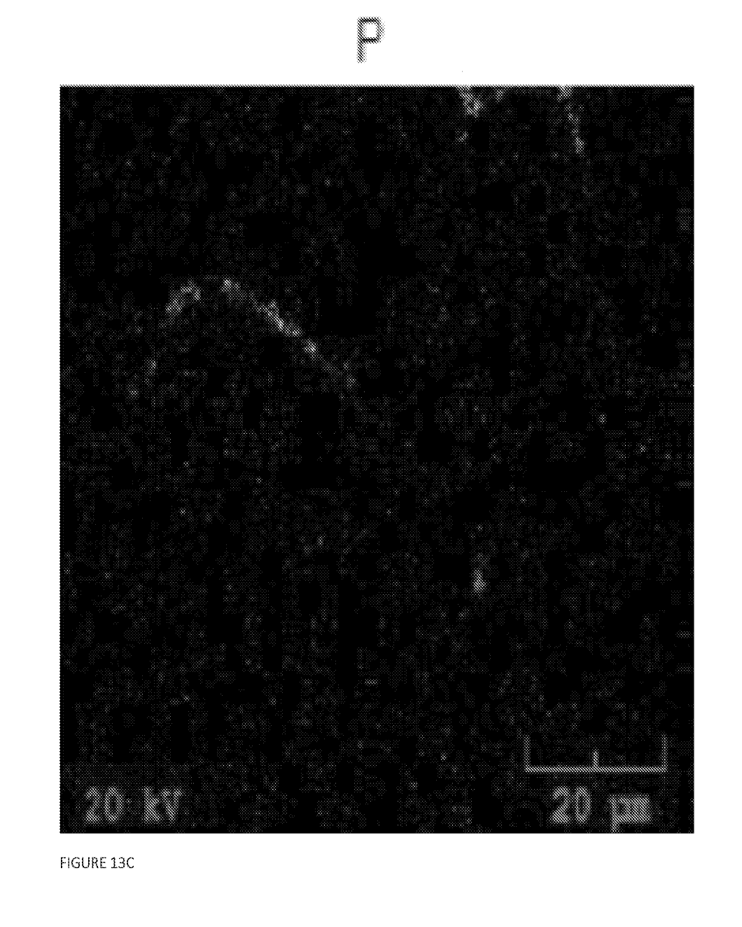

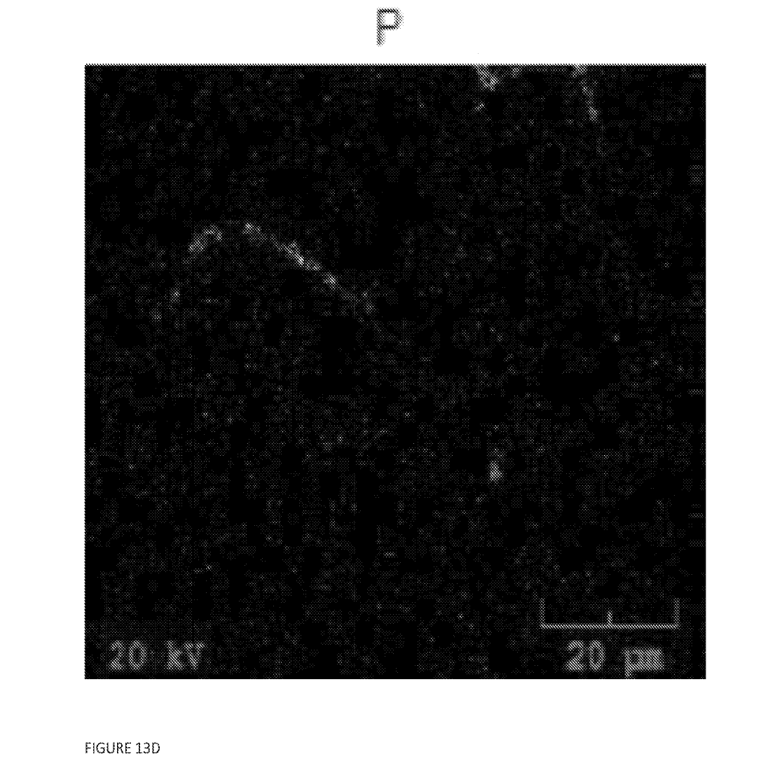

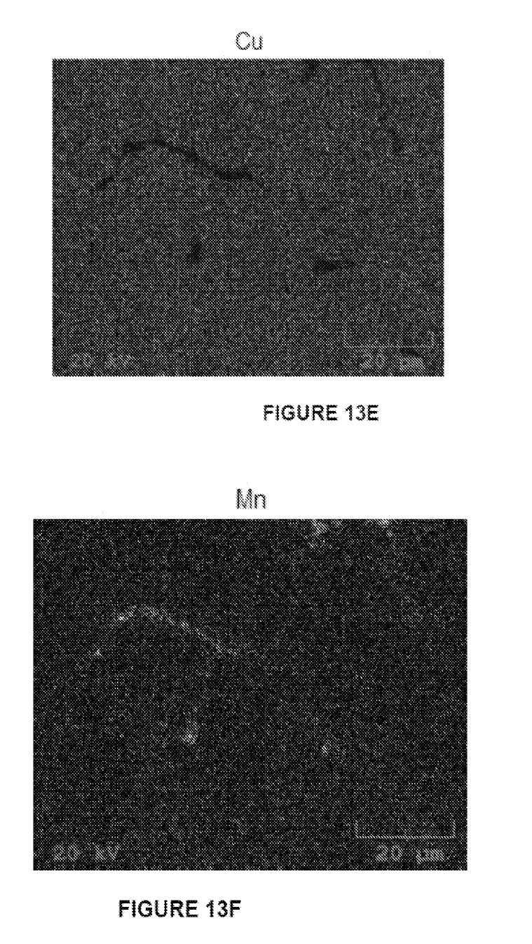

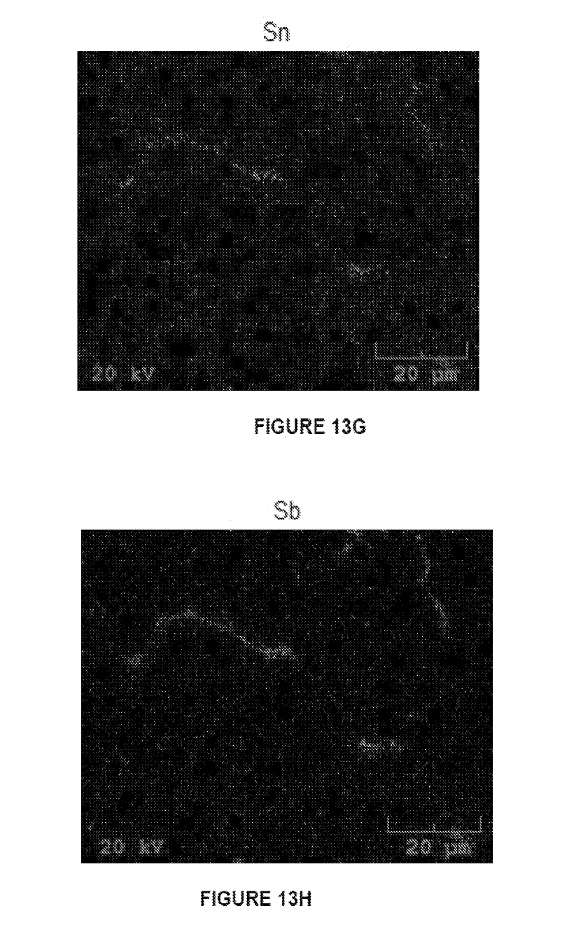

Element mapping of this same area is shown in FIGS. 13B-H. FIG. 13A is a SEM image of an embodiment of alloy C99761; FIG. 13B illustrates elemental mapping of sulfur in the portion shown in FIG. 13A; FIG. 13C illustrates elemental mapping of phosphorous in the portion shown in FIG. 13A; FIG. 13D illustrates elemental mapping of zinc in the portion shown in FIG. 13A; FIG. 13E illustrates elemental mapping of copper in the portion shown in FIG. 13A; FIG. 13F illustrates elemental mapping of manganese in the portion shown in FIG. 13A; FIG. 13G illustrates elemental mapping of tin in the portion shown in FIG. 13A; FIG. 13H illustrates elemental mapping of antimony in the portion shown in FIG. 13A. As can be seen in the observed samples consist of dispersed particles in a copper-rich matrix. Many of the other non-copper metals are located in distinct clusters.

Shrinkage porosity was noted throughout the material. Image analysis was performed on one 500× image (see FIG. 14C). The minimum, maximum, and average particle sizes are reported in the following table.

TABLE 12
C99761 Particle Size.

| | Minimum (μm) | Maximum (μm) | Average (μm) |
|---|---|---|---|
| Sample | <0.1 | 14.5 | 2.0 |

The backscattered electron images (FIGS. 14A and 14B for C99761) show a dendritic microstructure with some shrinkage porosity in the interdendritic areas. These are characteristics of long freezing range alloys. Phases present in the grain boundaries and interdentritic areas have been analysed by EDS as shown above for C99761.

### C99771

### Sand Cast

Metallography study was done for the alloy listed in FIG. 15A (69.2 Cu, 3.21 Ni, 8.10 Mn, 17.56 Zn, 0.014 S, 0.685 Sb, 0.319 Fe, 0.616 Sn, 0.006 Pb, 0.224 Al). Scanning electron microscopy (SEM) uses electrons for imaging, much as a light microscope uses visible light. Imaging was performed using secondary electrons (SE) for best resolutions of fine topographical features. Further imaging with backscattered electrons (BE) gives contrast based on atomic number to resolve microscopic composition variations, as well as topographical information. Qualitative and quantitative chemical analysis was performed using energy dispersive X-ray spectrometry (EDS) with the SEM. This instrument is equipped with a light element detector capable of detecting carbon and elements with a higher atomic number (i.e., cannot detect hydrogen, helium, lithium, beryllium, and boron). Each sample was mounted in conductive epoxy, metallographically prepared to a 0.04 μm finish, and examined using BE imaging to further identify observed particles.

FIG. 15B illustrates a BE image of an embodiment of C99771 alloy that is further analyzed at 5 discreet locations via SEM/EDS spectra. SEM/EDS spectra results of the base material the sample of C99771 consist of significant amounts of copper with lesser amounts of manganese, iron, nickel, and zinc (see Location 1, FIG. 15B). The light colored phase reveals antimony and tin in addition to manganese, iron, nickel, copper, and zinc (see Location 2, FIG. 15B). The dark gray colored phase reveals significant amounts of sulfur and manganese with lesser amounts of iron, nickel, copper, zinc, selenium, and antimony (see Location 3, FIG. 15B). The light gray colored phase at Location 4 reveals phosphorus, tin, and antimony in addition to manganese, iron, nickel, copper, zinc, and tin (see Location 4, FIG. 15B). Semi-quantitative chemical analysis data is reported in the following table for the above locations.

TABLE 13
C99771 Sand Cast EDS Spectra analysis.

| Spectrum | Si | P | S | Mn | Fe | Ni | Cu | Zn | Se | Sn | Sb |
|---|---|---|---|---|---|---|---|---|---|---|---|
| Location 1-Base | — | — | — | 5.8 | <1 | 3.4 | 73.5 | 17.0 | — | — | — |
| Location 2 | — | — | — | 21.8 | <1 | 13.6 | 12.8 | 1.0 | — | 3.8 | 46.6 |
| Location 3 | — | — | 24.5 | 52.1 | <1 | <1 | 16.0 | 3.5 | 1.1 | — | 1.4 |
| Location 4 | <1 | 5.3 | — | 27.3 | 1.6 | 10.8 | 24.7 | 5.1 | — | 2.2 | 22.7 |

Results in weight percent unless otherwise indicated.

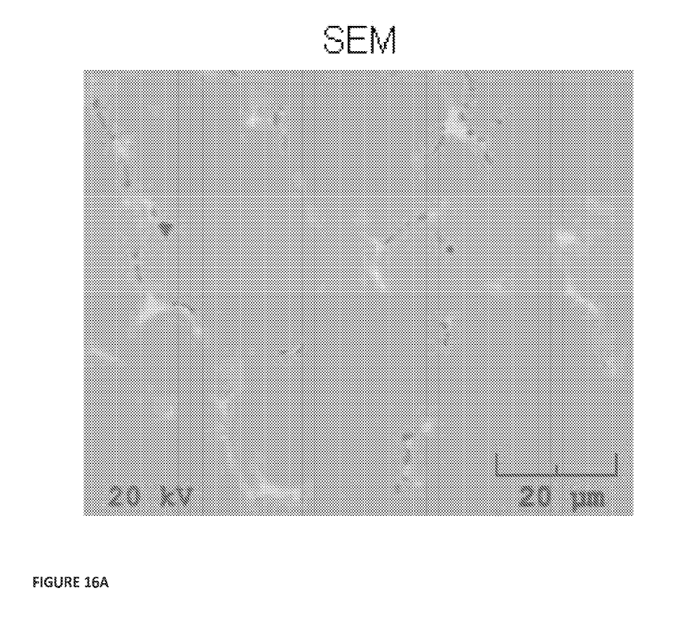

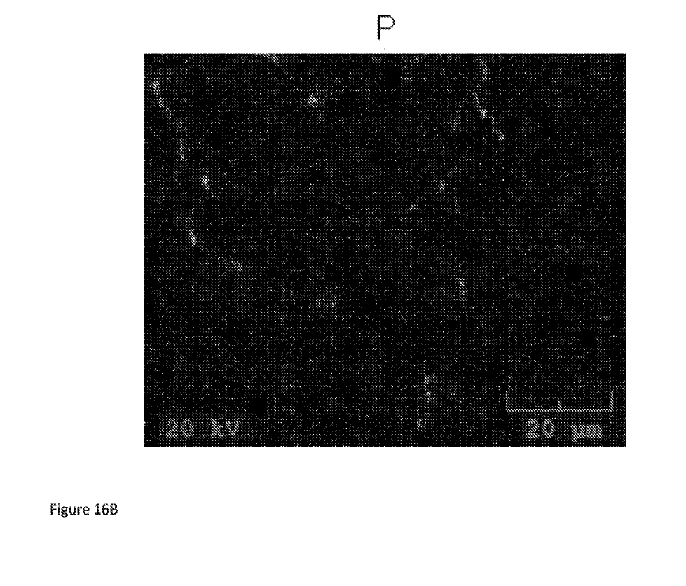

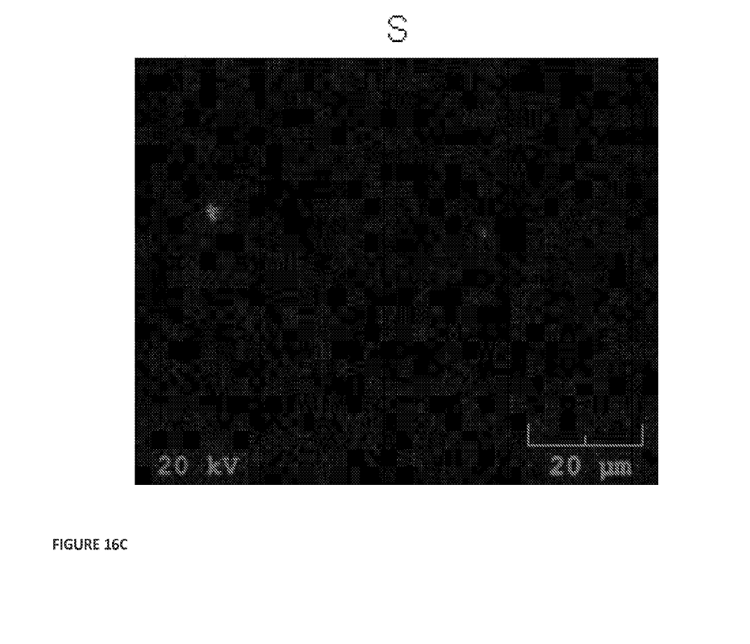

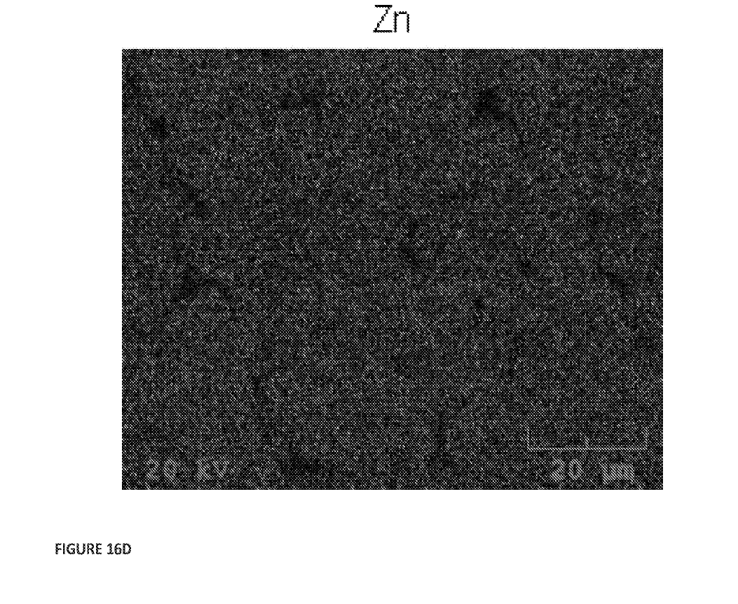

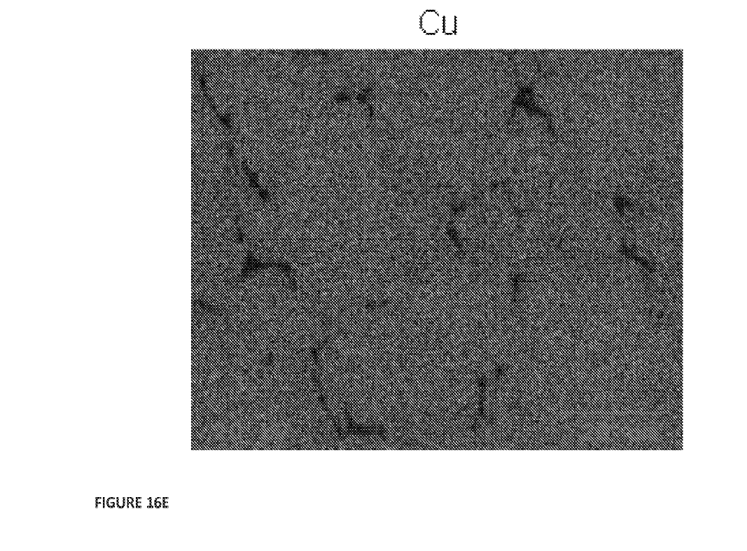

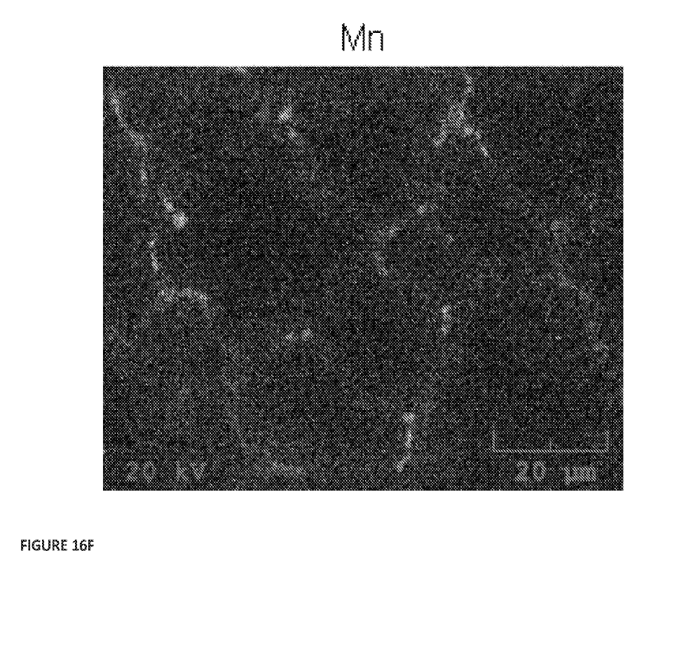

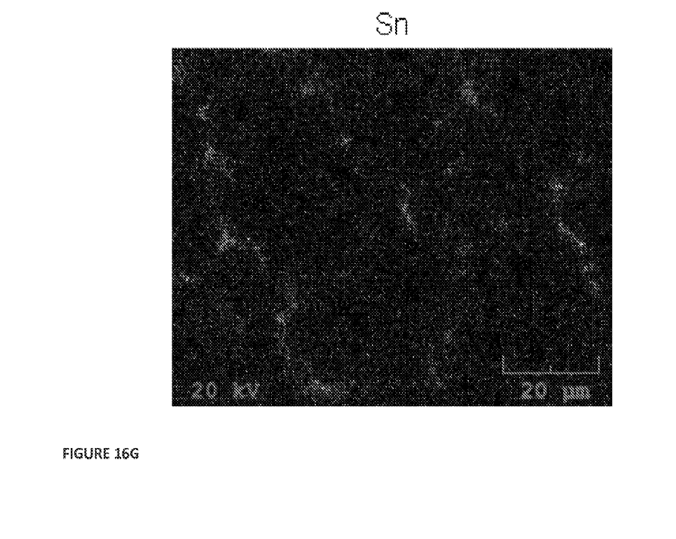

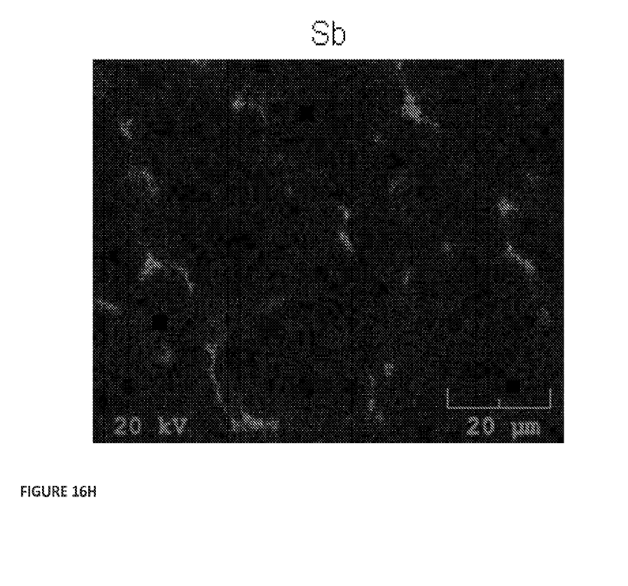

FIG. 16A is a SEM image of an embodiment of alloy C99771; FIG. 16B illustrates elemental mapping of phosphorous in the portion shown in FIG. 16A; FIG. 16C illustrates elemental mapping of sulfur in the portion shown in FIG. 16A; FIG. 16D illustrates elemental mapping of zinc in the portion shown in FIG. 16A; FIG. 16E illustrates elemental mapping of copper in the portion shown in FIG. 16A; FIG. 16F illustrates elemental mapping of manganese in the portion shown in FIG. 16A; FIG. 16G illustrates elemental mapping of tin in the portion shown in FIG. 16A; FIG. 16H illustrates elemental mapping of antimony in the portion shown in FIG. 16A. As can be seen in the observed samples consist of dispersed particles in a copper-rich matrix. Many of the other non-copper metals are located in distinct clusters.

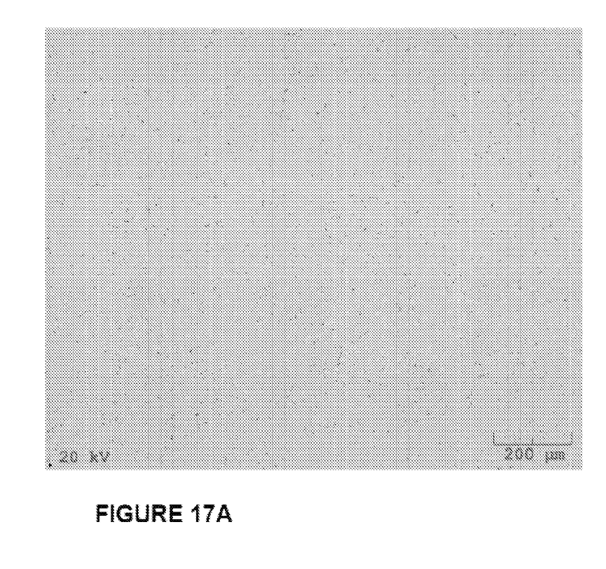

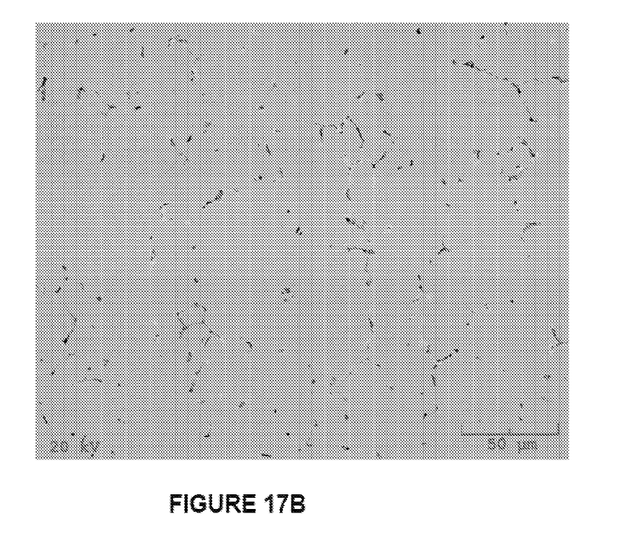

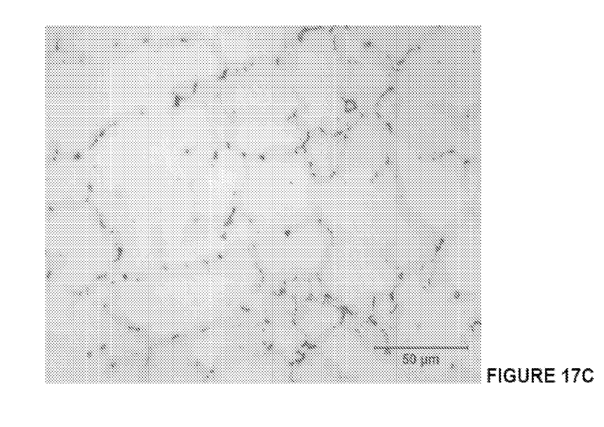

Representative BE images of the microstructure taken at 200× and 1000× are shown in FIGS. 17A and 17B, respectfully. BE imaging with EDS was performed to determine the chemistry of the various secondary phases present in the copper alloy. The observed samples consist of dispersed particles throughout the copper-rich matrix. Image analysis was then performed to determine particle size. The minimum, maximum, and average are reported in the following table. Image analysis for particle size was performed on micrographs found in FIG. 17C.

TABLE 14
C99771 Particle Size

| | Minimum | Maximum | Average |
|---|---|---|---|
| Sample ID | (μm) | (μm) | (μm) |
| Sample 3 | <0.1 | 10.6 | 1.3 |

The backscattered electron images (FIGS. 17A and 17B for C99771) show a dendritic microstructure with some shrinkage porosity in the interdendritic areas. These are characteristics of long freezing range alloys. Phases present in the grain boundaries and interdentritic areas have been analysed by EDS as shown above for C99771.

### C99761

### Permanent Mold

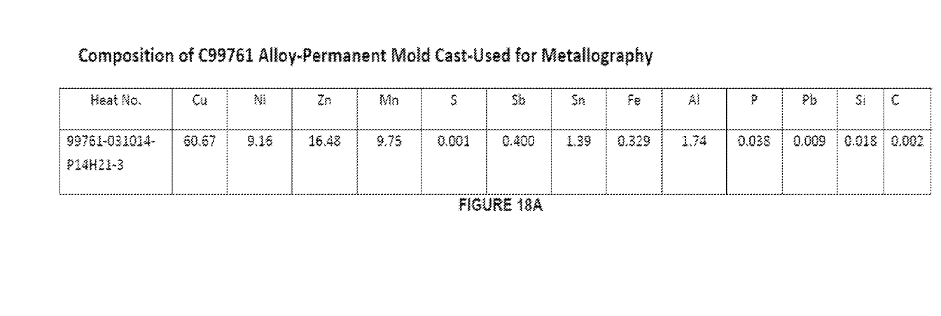

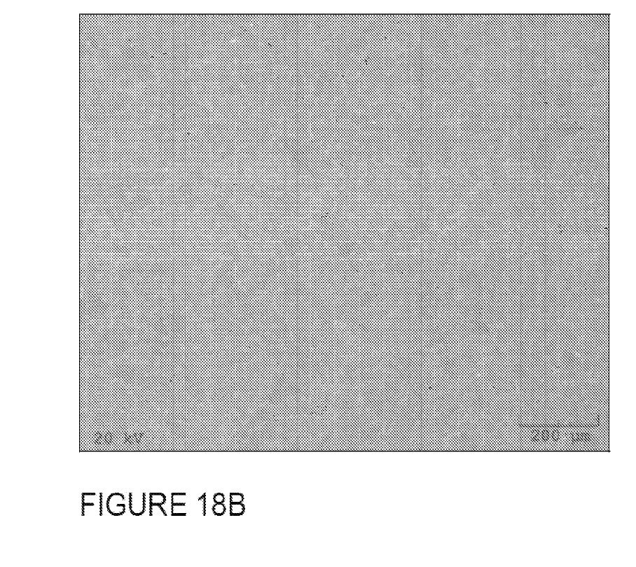

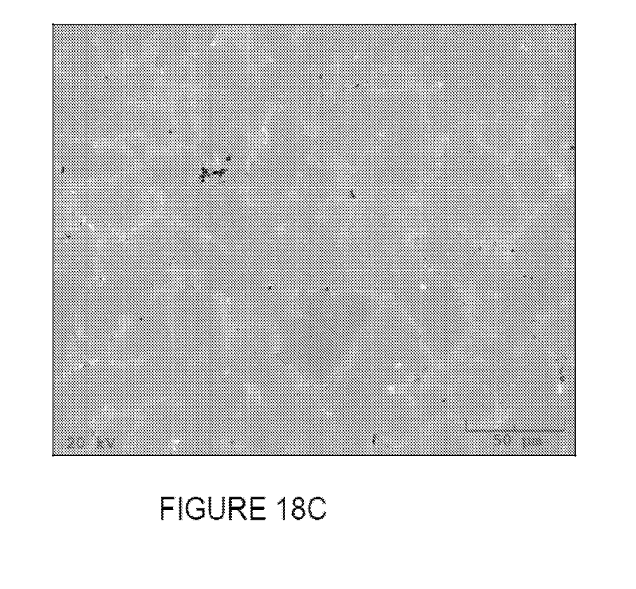

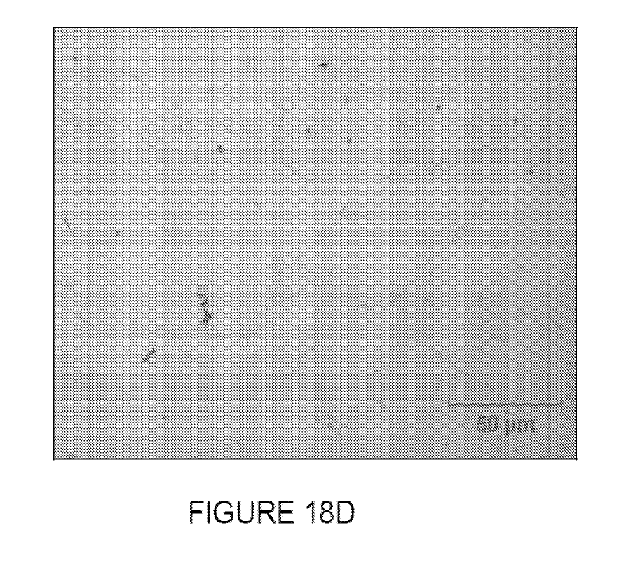

The C99761 Permanent Mold samples were examined using a scanning electron microscope with energy dispersive spectroscopy (SEM/EDS). This instrument is equipped with a light element detector capable of detecting carbon and elements with a higher atomic number (i.e., cannot detect hydrogen, helium, lithium, and beryllium, and boron detection is marginal). Images were acquired using the secondary electron (SE) and backscattered electron (BE) detectors. In backscattered electron imaging, elements with a higher atomic number appear brighter. The sample was examined using a 20 kV accelerating voltage. Representative BE images of the microstructure of a heat of C99761 listed in FIG. 18A taken at 200× and 1000× are shown in FIG. 18B-D respectively.

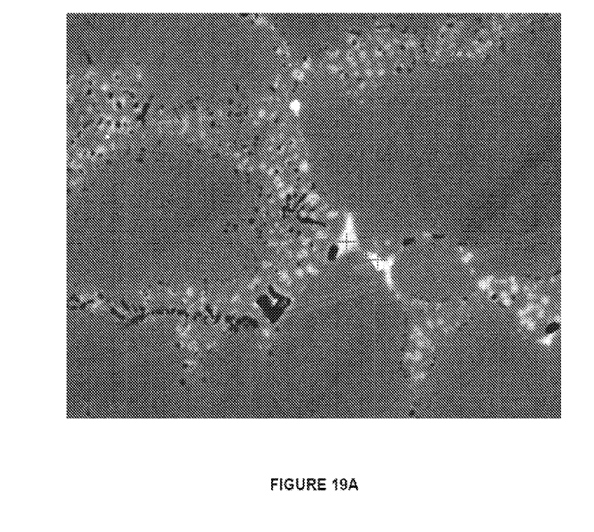

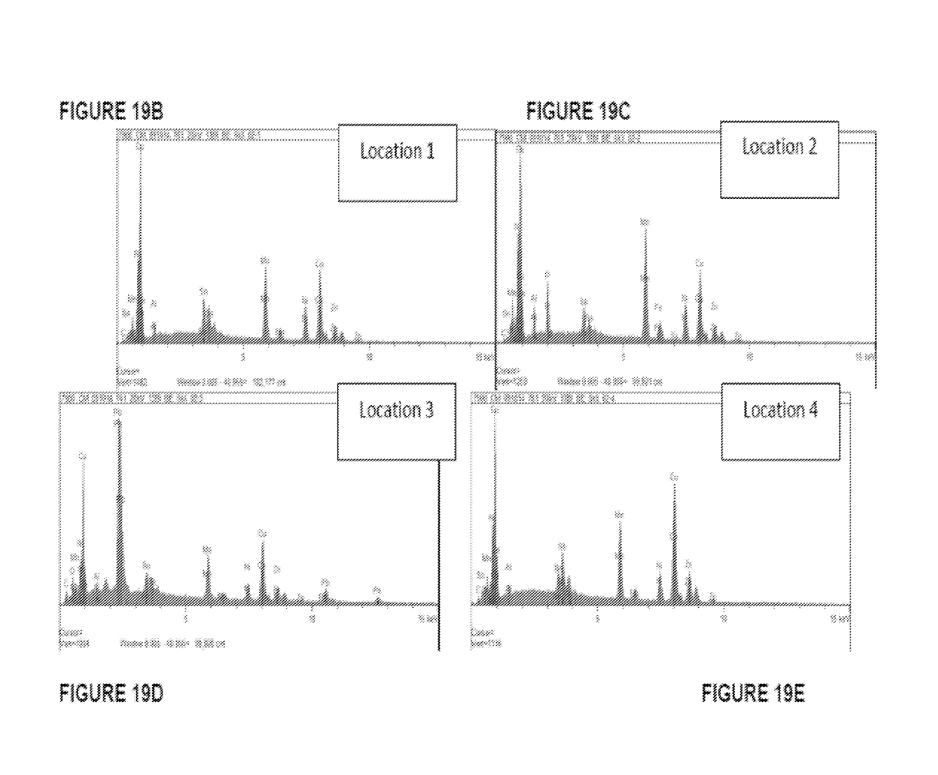

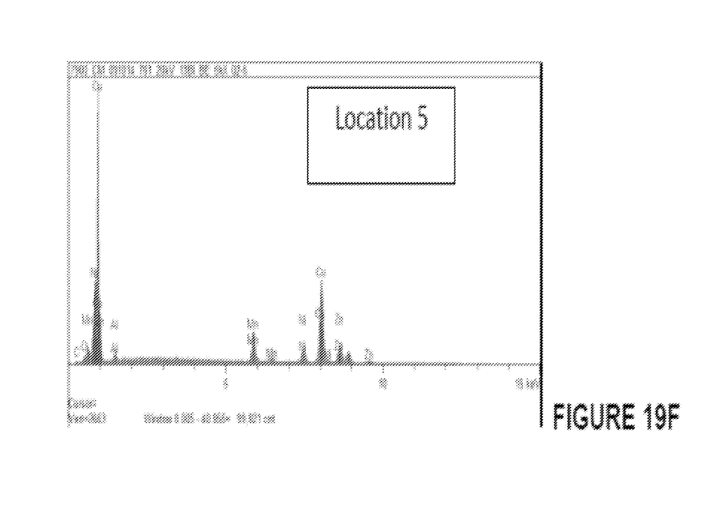

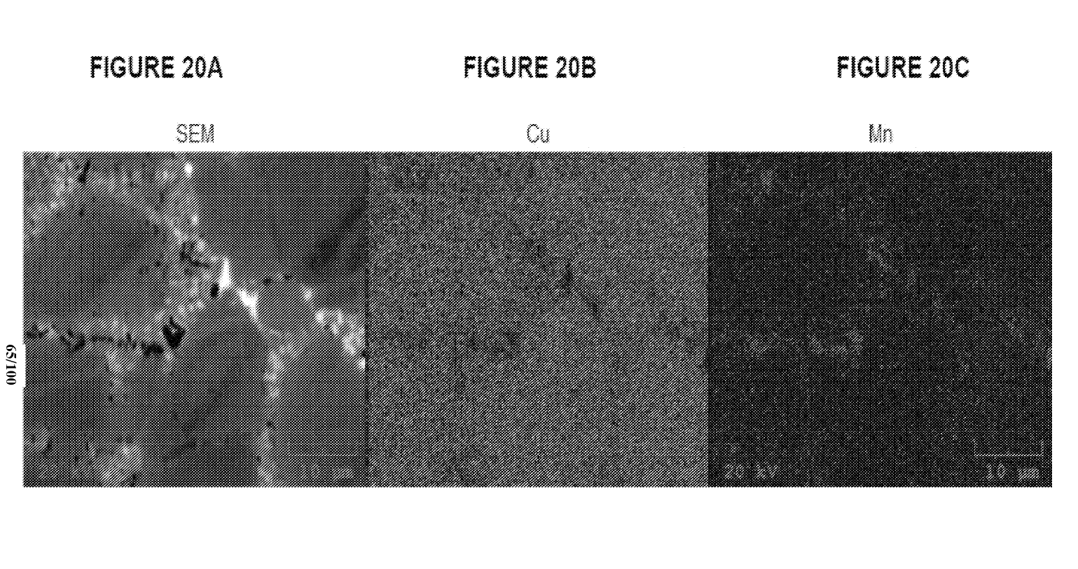

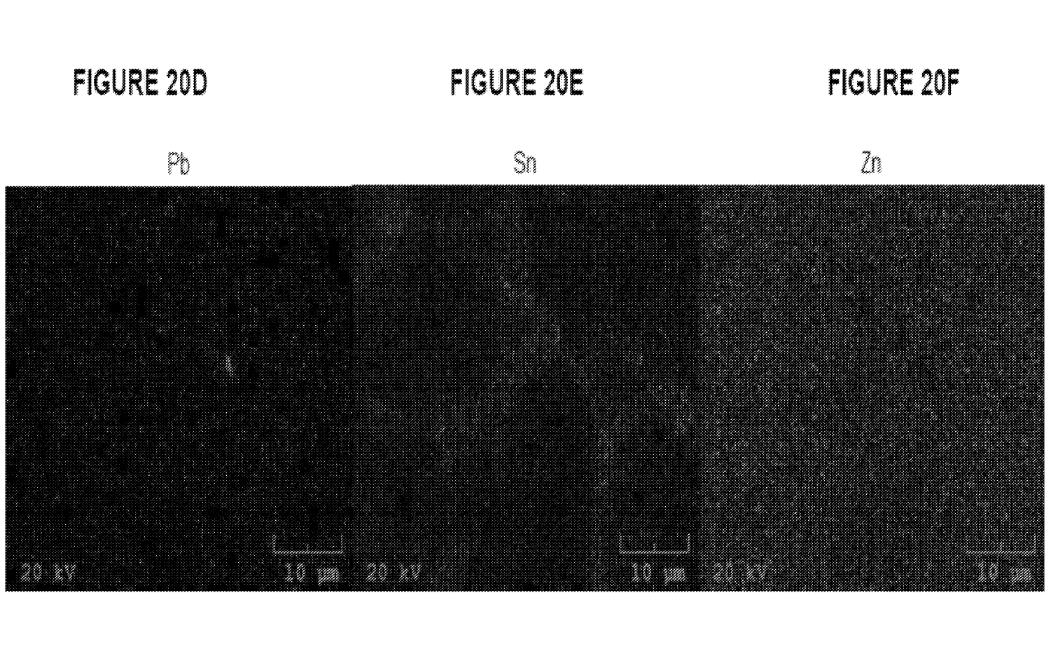

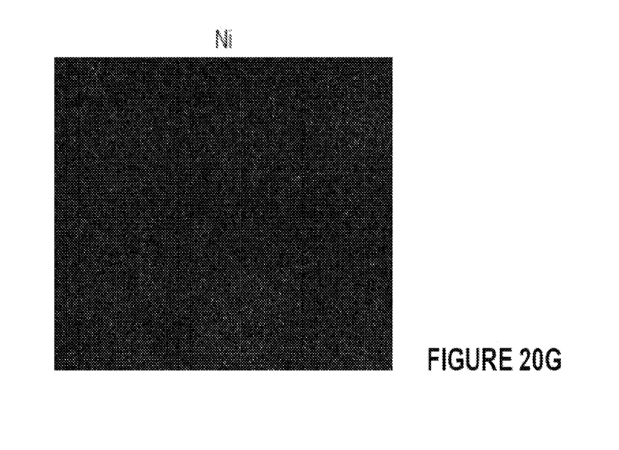

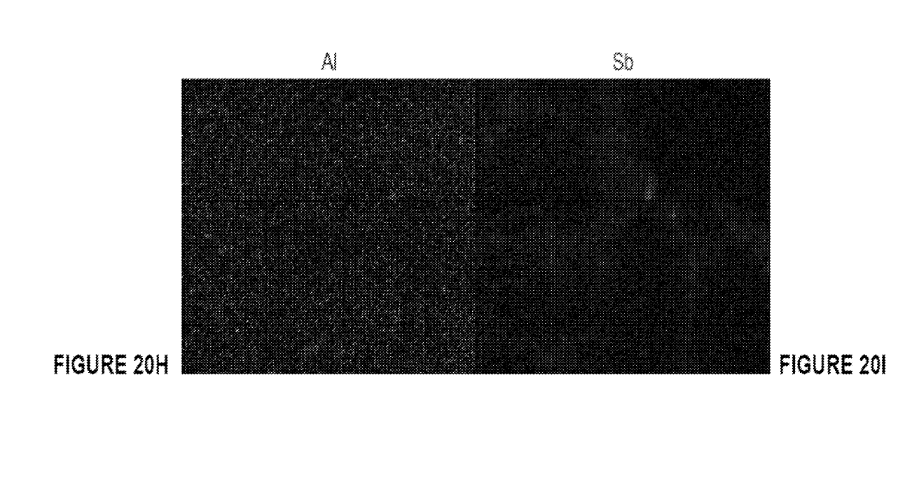

BE imaging with EDS was performed to determine the chemistry of the various secondary phases present in the copper alloy of a sample having the 99761 composition of FIG. 18A. FIG. 19 illustrates the BE image and the indicated locations for EDS. SEM/EDS spectra results of the base material from consist of high concentrations of copper with lesser amounts of manganese, nickel, aluminum, and zinc (see Location 5, FIG. 19F). The light gray colored phase reveals high concentrations of copper with lesser concentrations of aluminum, manganese, nickel, zinc, and tin (see Location 1, FIG. 19B). The dark colored phase reveals high concentrations of copper and manganese with lesser concentrations of aluminum, phosphorus, iron, nickel, zinc and tin (see Location 2, FIG. 19C). The bright white phase at Location 3 reveals high concentrations of lead with lesser concentrations of aluminum, manganese, nickel, copper, zinc, and tin (see Location 3, FIG. 19D). This region also showed some bismuth, which was not captured in this semi-quantitative analysis, but shows up in the element mapping. This alloy contains only 0.009% Pb. The high concentration of Pb at Location 1 indicates the entrapment of a lead particle. The light phase at Location 4 reveals high concentrations of copper with lesser amounts of aluminum, manganese, nickel, zinc, tin, and antimony (see Location 4, FIG. 19E). Semi-quantitative chemical analysis data is reported in the following table for the above locations.

TABLE 15
C99761 Perm. Mold EDS Spectra analysis.

| spectrum | Al | P | Mn | Fe | Ni | Cu | Zn | Sn | Sb | Pb |
|---|---|---|---|---|---|---|---|---|---|---|
| Location 1 | 2.0 | — | 17.3 | — | 15.2 | 42.0 | 9.5 | 14.0 | — | — |
| Location 2 | 3.4 | 4.7 | 23.5 | 1.6 | 15.0 | 35.1 | 9.2 | 7.4 | — | — |
| Location 3 | <1 | — | 7.7 | — | 4.4 | 22.0 | 5.5 | 6.9 | — | 53.0 |
| Location 4 | 1.1 | — | 13.3 | — | 8.9 | 48.9 | 13.7 | 5.4 | 8.8 | — |
| Location 5-Base | 2.1 | — | 9.3 | — | 8.8 | 63.8 | 16.0 | — | — | — |

The observed samples consist of dispersed particles in a copper-rich matrix. Shrinkage porosity was noted throughout the material. Image analysis was performed on one 500× image (see FIG. 18D). The minimum, maximum, and average particle sizes are reported in the following table.

TABLE 16
C99761 Perm. Mold EDS Spectra analysis.

| | | | Average |
|---|---|---|---|
| Sample | Minimum (μm) | Maximum (μm) | (μm) |
| 99761-031014-P14H21 | 0.1 | 40.2 | 1.7 |

### C99771

### Permanent Mold

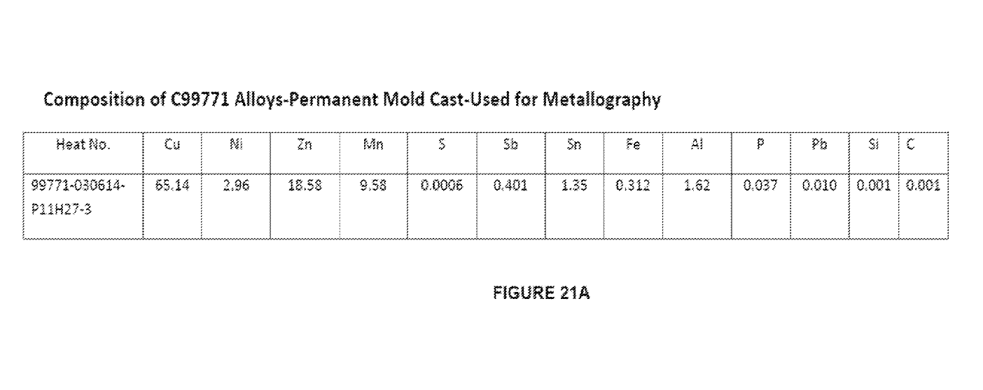

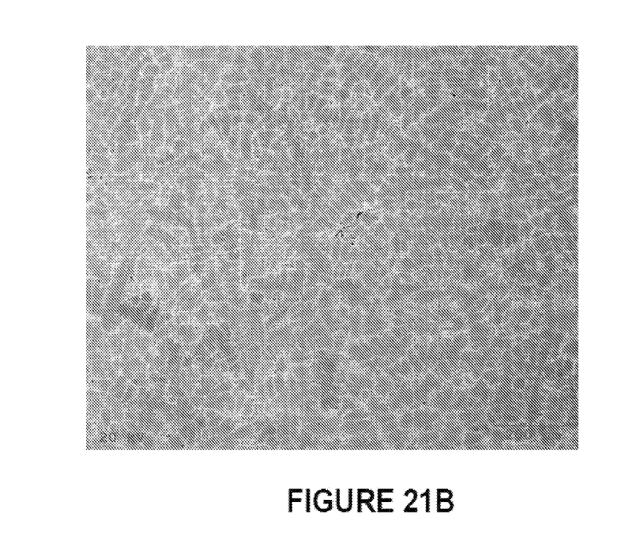

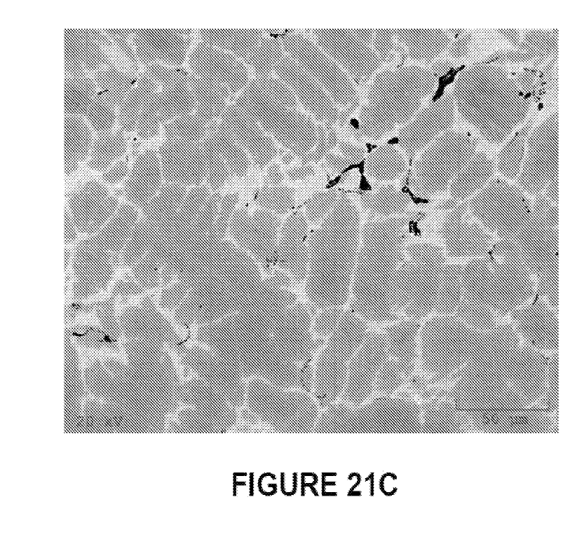

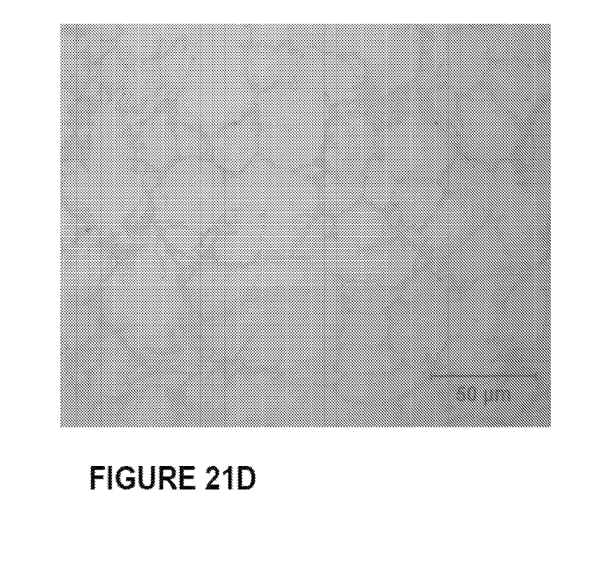

The C99771 Permanent Mold samples were examined using a scanning electron microscope with energy dispersive spectroscopy (SEM/EDS). This instrument is equipped with a light element detector capable of detecting carbon and elements with a higher atomic number (i.e., cannot detect hydrogen, helium, lithium, and beryllium, and boron detection is marginal). Images were acquired using the secondary electron (SE) and backscattered electron (BE) detectors. In backscattered electron imaging, elements with a higher atomic number appear brighter. The sample was examined using a 20 kV accelerating voltage. Representative BE images of the microstructure of a heat of C99771 (permanent mold) listed in FIG. 21A taken at 200× and 1000× are shown in 21B-C respectively.

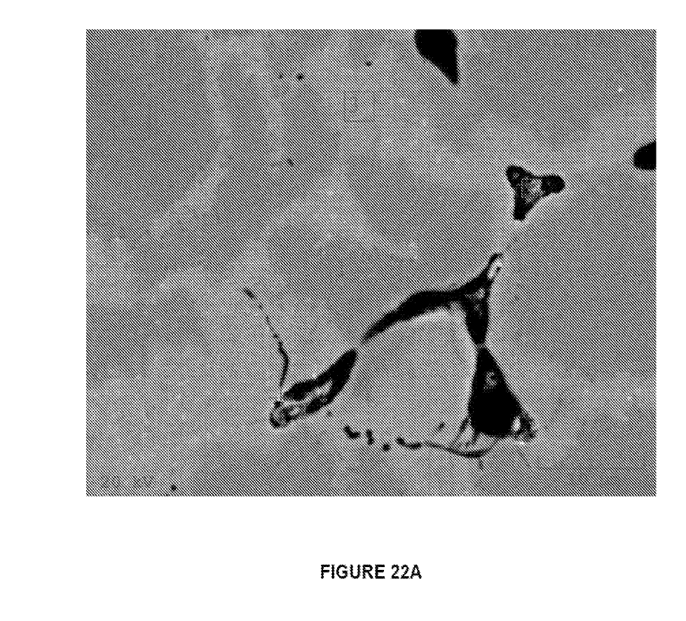

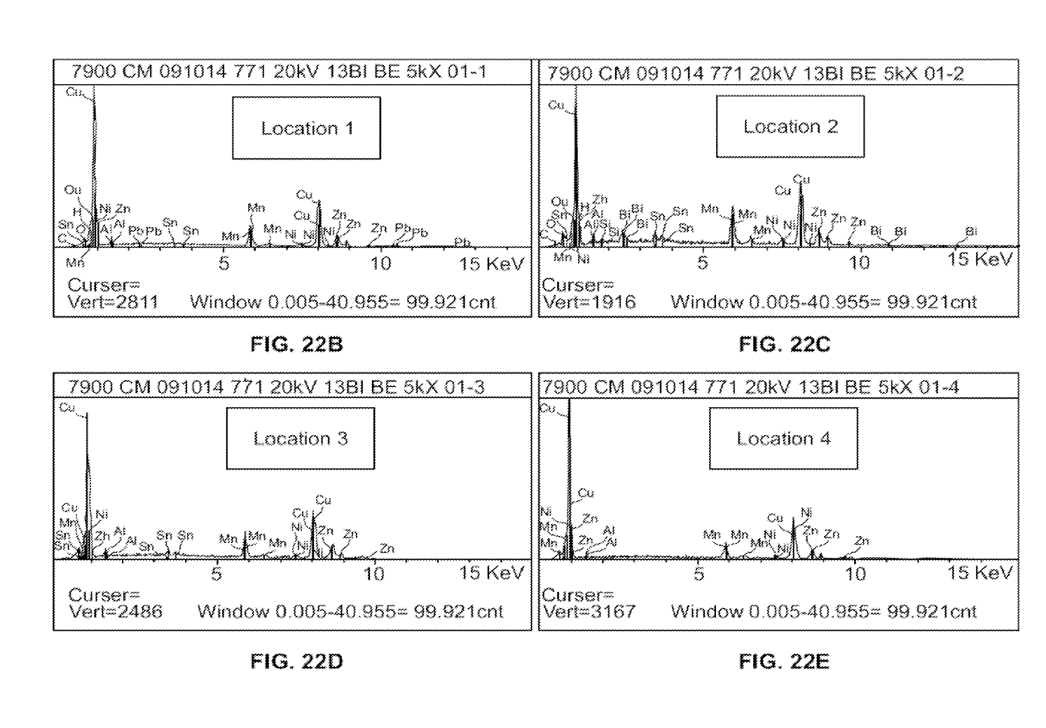

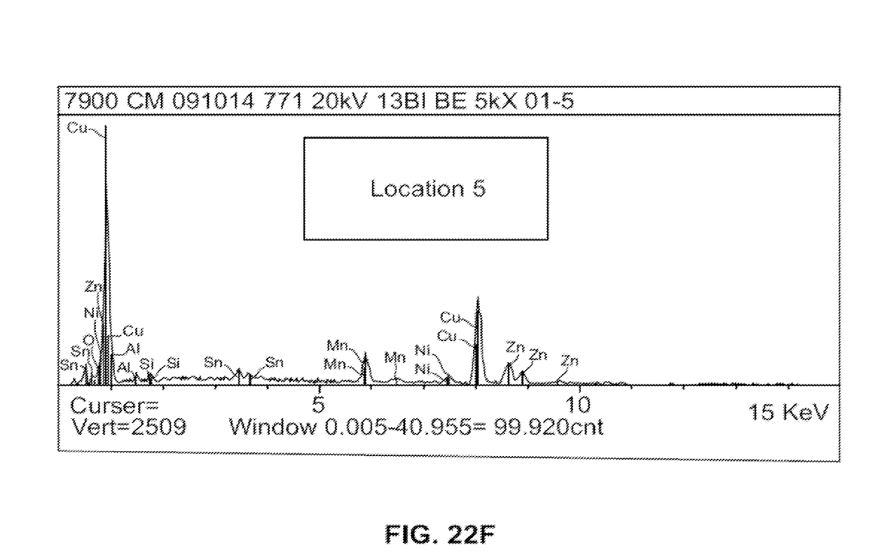

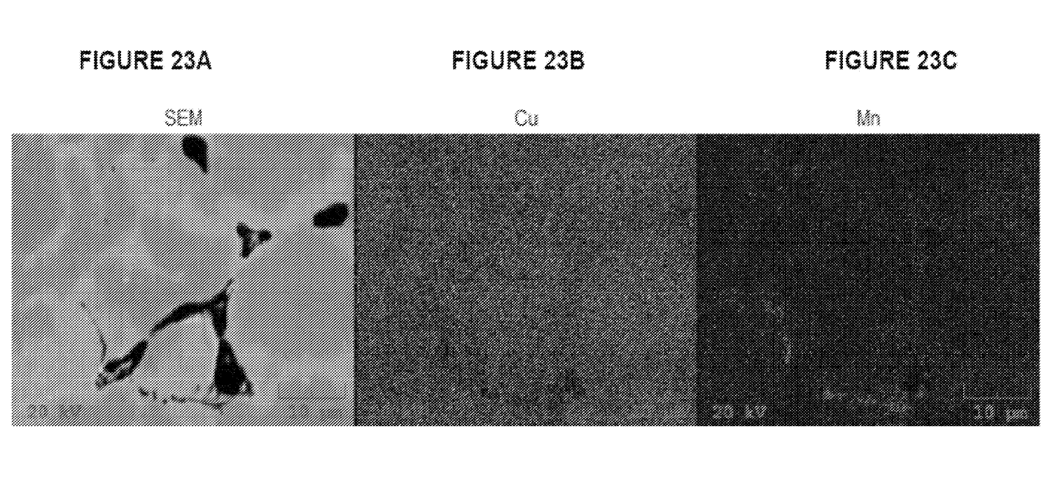

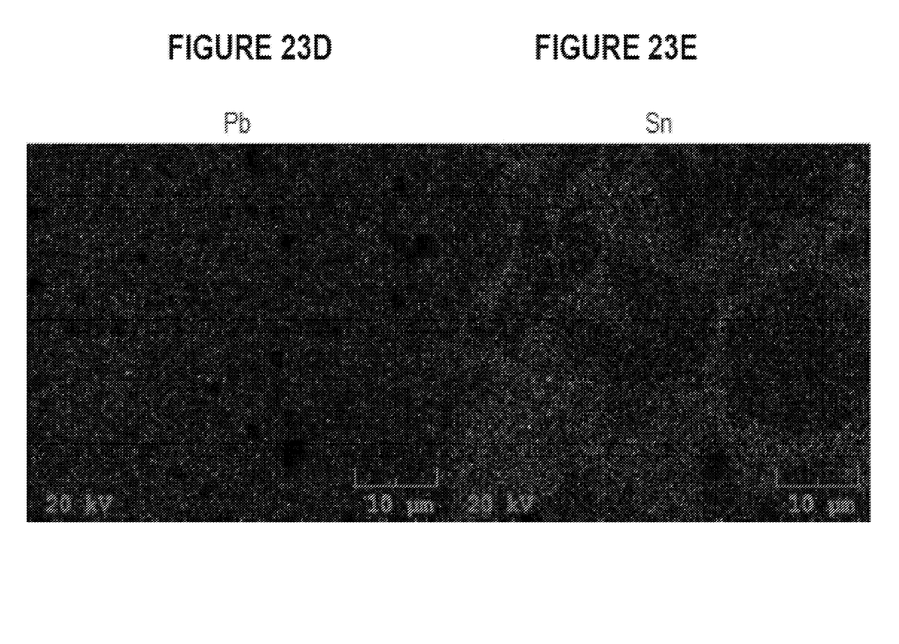

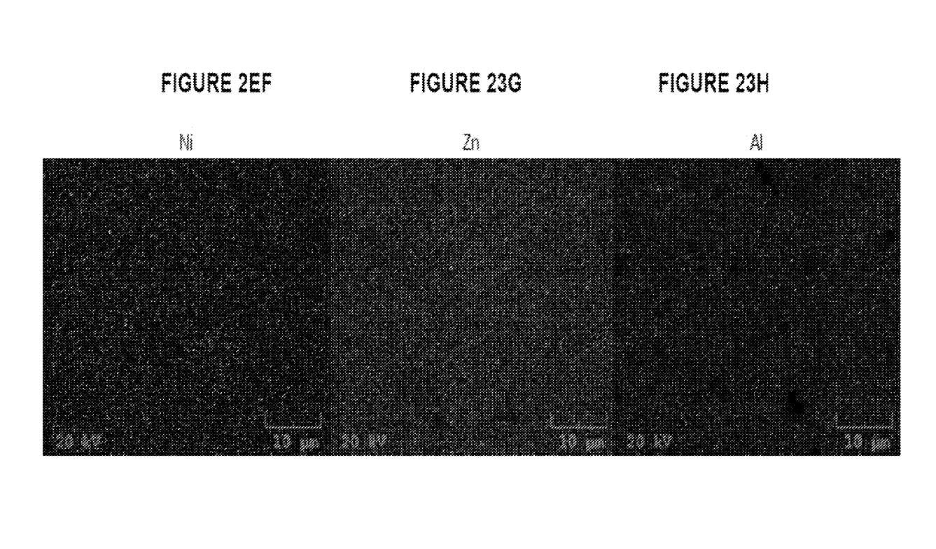

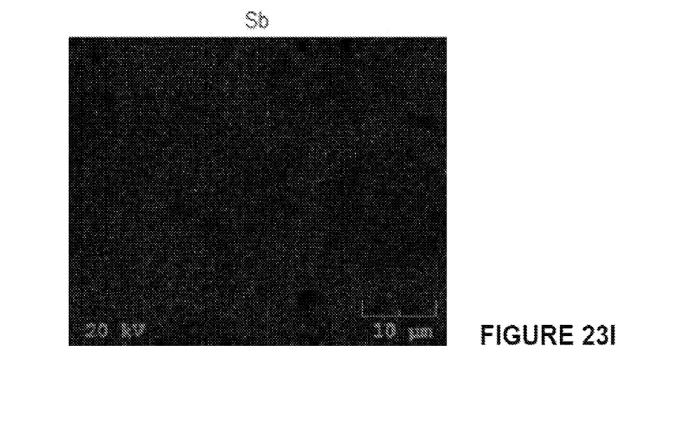

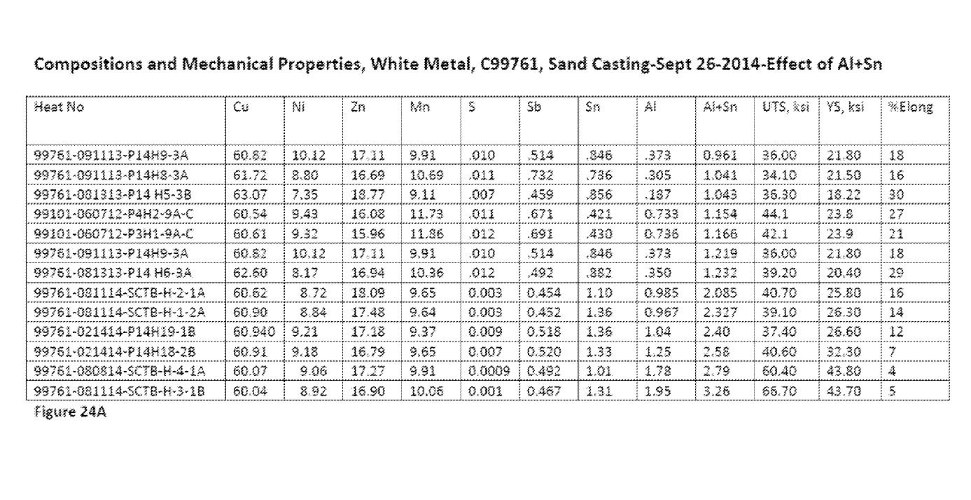

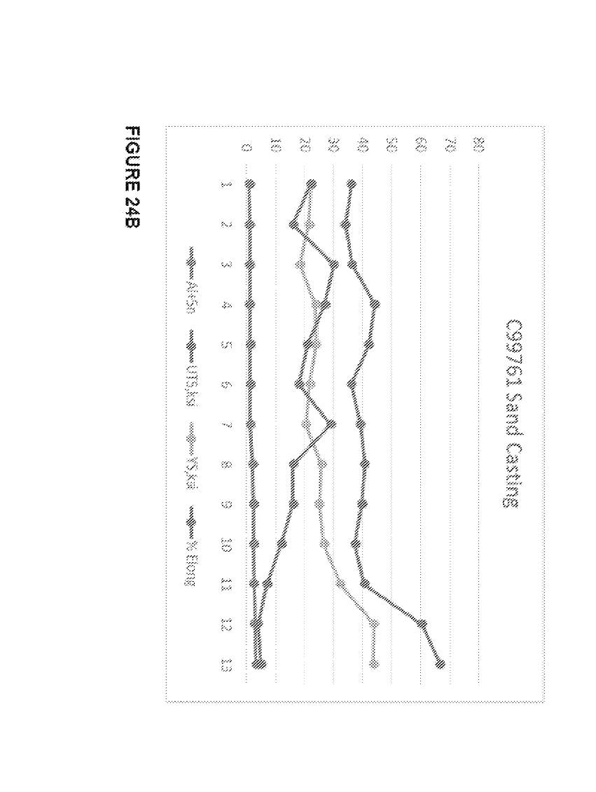

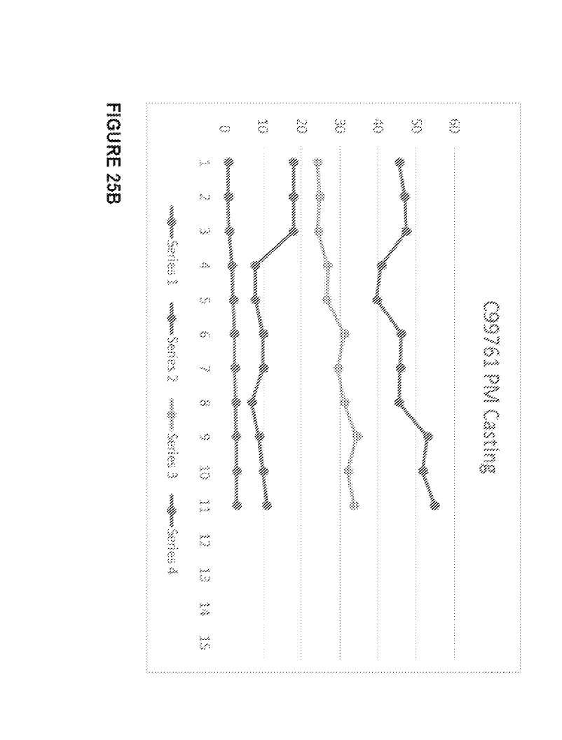

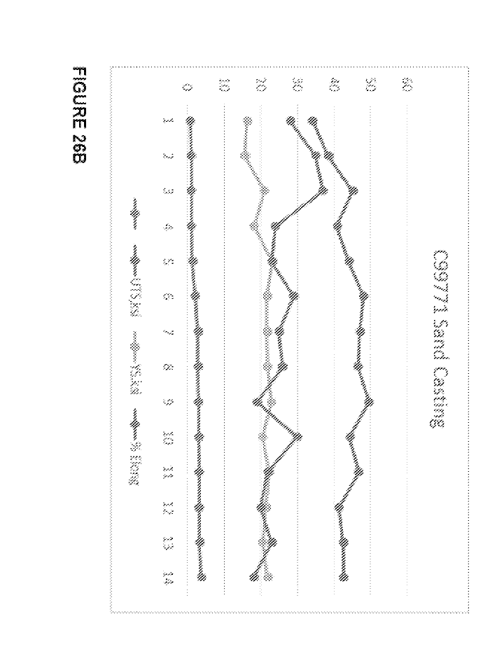

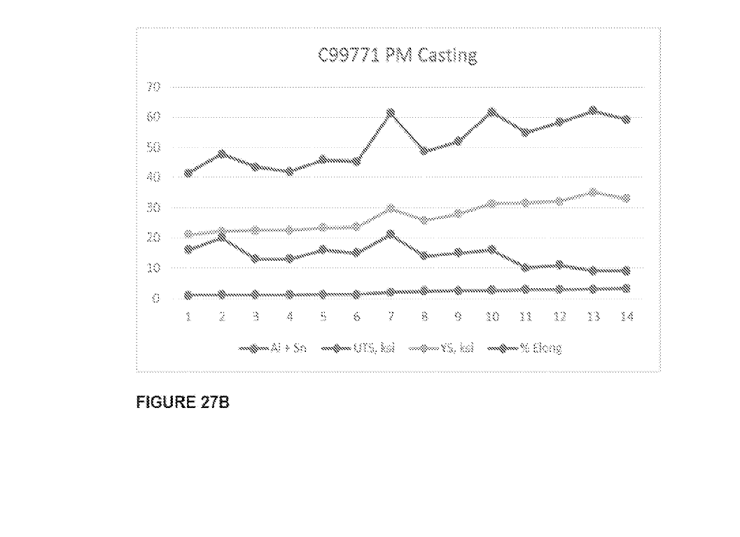

BE imaging with EDS was performed to determine the chemistry of the various secondary phases present in the copper alloy of C99771 of FIG. 21A. SEM/EDS spectra results of the base material from consist of high concentrations of copper with lesser amounts of aluminum, silicon, manganese, nickel, zinc and tin (see Location 4, FIG. 22E). The bright white colored phase reveals high concentrations of copper with lesser amounts of aluminum, manganese, nickel, zinc, tin, and lead (see Location 1, FIG. 22B). This alloy contains only 0.010% Pb. The high concentration of Pb at Location 1 indicates the entrapment of a lead particle. A second bright white colored phase reveals high concentrations of copper with lesser amounts of aluminum, silicon, manganese, nickel, zinc, tin, and bismuth (see Location 2, FIG. 22C). The lighter phase at Location 3 reveals high concentrations of copper with lesser concentrations of aluminum, manganese, nickel, zinc, and tin (see Location 3, FIG. 22D). The dark colored phase at Location 5 consists of high concentrations of copper with lesser amounts of aluminum, silicon, manganese, nickel, zinc and tin (see Location 5, FIG. 22F). This location appears similar to the base metal chemistry and is likely shrinkage porosity. Semi-quantitative chemical analysis data is reported in the following table for the above locations.

TABLE 17
C99771 Perm. Mold EDS Spectra analysis.

| Spectrum | Al | Si | Mn | Ni | Cu | Zn | Sn | Pb | Bi |
|---|---|---|---|---|---|---|---|---|---|
| Location 1 | 2.0 | — | 10 | 3 | 63.6 | 17.9 | 1.6 | 2 | — |
| Location 2 | 1.9 | <1 | 12.6 | 3.8 | 52.2 | 14.5 | 5.9 | — | 8.3 |
| Location 3 | 2.3 | — | 12.4 | 2.4 | 58.5 | 19.1 | 5.3 | — | — |
| Location 4-Base | 1.8 | — | 9.1 | 2.9 | 68.9 | 17.3 | — | — | — |
| Location 5 | 1.9 | 1.3 | 9.0 | 3.7 | 62.9 | 17.0 | 4.3 | — | — |

The observed samples consist of dispersed particles in a copper-rich matrix. Shrinkage porosity was noted throughout the material. In C99771 of 21A, the majority of the second phase consists of a nearly continuous eutectic. Image analysis was performed on one 500× image (see FIG. 21D). The minimum, maximum, and average particle sizes are reported in the following table.

TABLE 18
C99771 Perm. Mold EDS Spectra analysis.

| Sample | Minimum (μm) | Maximum (μm) | Average (μm) |
|---|---|---|---|
| 99771-030614- | 0.1 | 196.1 | 2.4 |
| P11 H27 | | | |

### Color Comparison

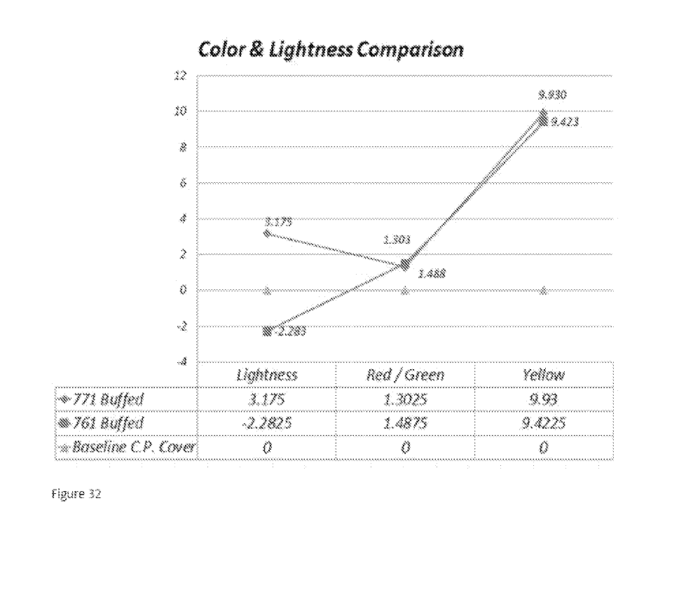

One novel aspect of the C99761 and C99771 alloys is their ability to provide the above described antimicrobial properties with the desired mechanical properties white exhibiting a white or sliver color. A study was done to compare C99761 and C99771 with a hexavalent chrome plated (CP) part. To this end, a standard hexavalent chrome plated (CP) cover is used. This is established as the zero that the tests are based on. FIG. 32 shows a comparison with the baseline cover, the lightness, red or green value, and blue or yellow values for buffed C99761 and C99771. These data show that alloy C99761 is only 3.18 units darker from the CP part, 1.35 units redder and 9.93 units yellower. These data show that alloy C99771 is only 2.28 units lighter from the CP part, 1.49 units redder and 9.42 units yellower. Since white metals will be used in the buffed condition, these data indicate that the two white metals compare favorably with respect to the CP cover.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

We claim:

1. A composition of matter consisting essentially of:
at least 60 wt % copper;
8 wt %-10 wt % nickel;
16 wt %-21 wt % zinc;
8 wt %-12 wt % manganese;
0.1 wt %-1 wt % antimony;
0.2 wt %-1.5 wt % tin;
0.1 wt %-2.0 wt % aluminum;
greater than 0 wt % and less than 0.25 wt % sulfur;
greater than 0 wt % and less than 0.6 wt % iron;
greater than 0 wt % and less than 0.1 wt % carbon;
greater than 0 wt % and less than 0.05 wt % phosphorous;
greater than 0 wt % and less than 0.09 wt % lead; and
greater than 0 wt % and less than 0.05 wt % silicon.

2. The composition of matter of claim 1, wherein the total wt % of tin and aluminum is about 1.5%.

3. The composition of claim 1, wherein the total wt % of tin and aluminum is greater than 2.5%.

4. A composition of matter consisting essentially of:
58 wt % to 64 wt % copper;
8 wt %-10 wt % nickel;
16 wt %-21 wt % zinc;
8 wt %-12 wt % manganese;
0.1 wt %-1 wt % antimony;
0.2 wt %-1.5 wt % tin;
0.1 wt %-2.0 wt % aluminum;
greater than 0 wt % and less than 0.25 wt % sulfur;
greater than 0 wt % and less than 0.6 wt % iron;
greater than 0 wt % and less than 0.1 wt % carbon;
greater than 0 wt % and less than 0.05 wt % phosphorous;
greater than 0 wt % and less than 0.09 wt % lead; and
greater than 0 wt % and less than 0.05 wt % silicon.

5. The composition of matter of claim 4, wherein the total wt % of tin and aluminum is about 1.5%.

6. The composition of claim 4, wherein the total wt % of tin and aluminum is greater than 2.5%.

7. A composition of matter comprising:
58 wt %-64 wt % copper
8 wt %-10 wt % nickel;
16 wt %-21 wt % zinc;
8 wt %-12 wt % manganese;
0.1 wt %-1 wt % antimony;
0.2 wt %-1.5 wt % tin; and
0.1-2.0 wt % aluminum.

8. The composition of matter of claim 7, further comprising greater than 0 wt % and less than 0.6 wt % iron.

9. The composition of matter of claim 7, further comprising greater than 0 wt % and less than 0.1 wt % carbon.

10. The composition of matter of claim 7, further comprising greater than 0 wt % and less than 0.05 wt % phosphorous.

11. The composition of matter of claim 7, further comprising greater than 0 wt % and less than 0.09 wt % lead.

12. The composition of matter of claim 7, further comprising greater than 0 wt % and less than 0.05 wt % silicon.

13. The composition of matter of claim 7, further wherein the total wt % of tin and aluminum is about 1.5%.

14. The composition of claim 7, wherein the total wt % of tin and aluminum is greater than 2.5%.

15. A composition of matter consisting essentially of:
62 wt % to 70% copper;
2 wt %-4 wt % nickel;
16 wt %-21 wt % zinc;
8 wt %-12 wt % manganese;
0.1 wt %-1.0 wt % antimony;
0.2 wt %-1.5 wt % tin;
0.1 wt %-2.0 wt % aluminum;
greater than 0 wt % and less than 0.25 wt % sulfur;
greater than 0 wt % and less than 0.6 wt % iron;
greater than 0 wt % and less than 0.1 wt % carbon;
greater than 0 wt % and less than 0.05 wt % phosphorous;
greater than 0 wt % and less than 0.09 wt % lead; and
greater than 0 wt % and less than 0.05 wt % silicon.

16. A composition of matter comprising:
62 wt %-70 wt % copper;
2 wt %-4 wt % nickel;
16 wt %-21 wt % zinc;
8 wt %-12 wt % manganese;
0.25 wt % sulfur;
0.1 wt %-1 wt % antimony;
0.2 wt %-1.5 wt % tin; and
0.1-2.0 wt % aluminum.

17. The composition of matter of claim 16, further comprising greater than 0 wt % and less than 0.6 wt % iron.

18. The composition of matter of claim 16, further comprising greater than 0 wt % and less than 0.1 wt % carbon.

19. The composition of matter of claim 16, further comprising greater than 0 wt % and less than 0.05 wt % phosphorous.

20. The composition of matter of claim 16, further comprising greater than 0 wt % and less than 0.09 wt % lead.

21. The composition of matter of claim 16, further comprising greater than 0 wt % and less than 0.05 wt % silicon.
